(19) United States
(12) Patent Application Publication
(10) Pub. No.: US 2025/0145619 A1
(43) Pub. Date: May 8, 2025

(54) KHK INHIBITOR, PREPARATION METHOD THEREFOR AND USE THEREOF

(71) Applicant: YOUNGENE THERAPEUTICS CO., LTD, Shanghai (CN)

(72) Inventors: Shougang HU, Shanghai (CN); Chenghai LI, Shanghai (CN)

(21) Appl. No.: 18/837,104

(22) PCT Filed: Jan. 30, 2023
(86) PCT No.: PCT/CN2023/073787

§ 371 Date: Aug. 8, 2024

(30) Foreign Application Priority Data
Feb. 9, 2022 (CN) 202210123398.2

(51) Int. Cl. C07D 471/04; A61K 31/4436; A61K 31/4439; A61K 31/4985; A61K 31/517; C07B 59/00; C07D 401/14; C07D 403/14; C07D 487/10; C07D 495/10

(52) U.S. Cl. CPC C07D 471/04; A61K 31/4436; A61K 31/4439; A61K 31/4985; A61K 31/517; C07B 59/002; C07D 401/14; C07D 403/14; C07D 487/10; C07D 495/10

(57) ABSTRACT

The present invention relates to a KHK inhibitor, a preparation method therefor, and a use thereof. In particular, the present invention relates to a KHK inhibitor having a structure of formula (I), a preparation method therefor, a pharmaceutical composition containing same and a use thereof as a KHK inhibitor as well as a use thereof for treatment and/or prevention of KHK-related diseases. Each substituent group of the formula (I) is as defined in the specification.

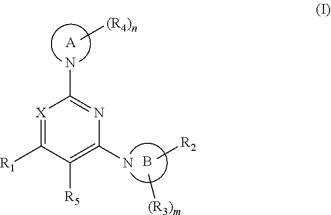

### TECHNICAL FIELD

The present invention is generally related to pharmaceutical synthesis, in particular, to a KHK inhibitor, a preparation method therefor and use thereof.

### BACKGROUND

The prevalence of metabolic diseases such as obesity, diabetes, fatty liver, and hyperlipidemia is increasing rapidly across the world. These metabolic diseases can further develop into cardiovascular diseases such as atherosclerosis, hypertension, and coronary heart disease, or various other diseases such as kidney diseases and gout, seriously endangering people's physical and mental health and social development.

Plenty of epidemiological data and experimental studies have shown that excessive fructose intake and abnormal metabolic synthesis may be important factors contributing to the increased incidence of metabolic diseases. The extensive application of high-fructose corn syrup has greatly increased the intake of fructose by modern people. The excessive fructose intake may cause accumulation of visceral fat to induce obesity, hyperlipidemia, hypertension, insulin resistance, and hyperuricemia, resulting in metabolic diseases such as diabetes, fatty liver, gout, and acute and chronic kidney disease. Of these metabolic diseases, the most common concern is non-alcoholic fatty liver disease (NAFLD)/non-alcoholic steatohepatitis (NASH).

Recent studies have found that a diet high in fructose is an important cause of NASH. Many foods, including natural fruits, are rich in fructose. Due to the increasing amount of sugars (usually sucrose and high-fructose corn syrup) added in beverages and processed foods, the amount of fructose in the diet of modern people increases. High fructose intake has been proven to cause a lot of adverse metabolic effects, and plays a role in the development of obesity and metabolic syndrome, such as weight gain, hyperlipidemia, hypertension, and insulin resistance. The fructose contributes to the occurrence and progression of NAFLD, and exacerbates the development and deterioration of NAFLD. Meanwhile, high fructose intake increases the risk of NASH and advanced liver fibrosis. The westernized dietary habits and lifestyle has led to a significantly increased incidence of NAFLD. Epidemiological studies have found that about 10-20% of NAFLD patients will develop NASH, which is the result of the further development of simple fatty liver. Its pathological manifestations include lipid deposition, inflammatory cell infiltration, liver tissue necrosis, and fibrotic lesions. Across the world, the incidence of NASH in the general population is 5-7%, and significantly increases to 22% in the diabetic population. 15-25% of NASH patients will further develop into more severe liver cirrhosis or even hepatocellular carcinoma (HCC). NAFLD not only affects the hepatobiliary system of patients, but is also closely related to insulin resistance, blood lipid disorders, atherosclerosis, fat embolism, and hematologic system diseases. NASH is currently the second leading cause of liver transplantation in the United States, and there is currently no drug approved for the treatment of NASH.

In addition to the exogenous fructose intake from food, endogenous fructose can also be synthesized by a polyol pathway. In the human body, glucose is converted into fructose through the intermediate sorbitol by means of the polyol pathway to produce the endogenous fructose. Studies have found that the endogenous polyol pathway of fructose contributes to hyperglycemia; and in the presence of inflammation and other pathological environments, such as acute kidney injury, the activity of this pathway significantly increases; and the increase of endogenous fructose may be associated with the onset of a variety of diseases such as diabetic nephropathy and gout.

Although fructose and glucose are exactly the same in molecular weight, they differ greatly in metabolic pathway in vivo. Unlike glucokinase, fructokinase is not under negative feedback control by the substrate. As a result, the fructose taken up by cells can be rapidly phosphorylated, and in the case of excessive fructose intake, the rapid phosphorylation of fructose can consume a large amount of ATP and phosphoric acid. The reduction of phosphate groups stimulates AMP deaminase (AMPD), which catalyzes the degradation of AMP into hypoxanthylic acid, increasing the degradation rate of purines. Under the action of xanthine oxidoreductase (xanthine dehydrogenase and xanthine oxidase), hypoxanthine is oxidized into xanthine and eventually converted to uric acid. Unlike rodents, humans are congenitally deficient in uricase, so excessive fructose intake can promote the production of uric acid, leading to elevated levels of serum uric acid.

Ketohexokinase (KHK), also known as fructose kinase (FK), is a key enzyme in fructose metabolism. In the liver, KHK, with the assistance of adenosine triphosphate (ATP), phosphorylates the Ci site of fructose to produce fructose-1-phosphate (FiP), which enters the normal metabolic pathway; and meanwhile, uric acid is produced downstream the ATP. Two alternative mRNA spliceosome-expressed human ketohexokinases (hKHKs) encode two different regional isomerases KHK-A and KHK—C. KHK—C has a lower Km value, a higher Kcat, and catalytic efficiency more than 405 times higher, indicating that KHK—C has a significantly higher affinity and ability for fructose phosphorylation than KHK-A. Although KHK-A is widely expressed and KHK—C is distributed in the liver, kidney, and intestine, KHK—C is the main metabolic site of fructose in vivo.

KHK is a rate-limiting enzyme for metabolizing fructose into fructose-1-phosphate, and an important target for regulating fructose metabolism. Studies have shown that the mice with the knockout of the gene KHK crucial for fructose metabolism are protected from glucose-induced weight gain, insulin resistance, and steatosis, indicating that in hyperglycemic cases, endogenous fructose production can promote insulin resistance and steatosis. Fructose is the only common carbohydrate that produces uric acid during its metabolism, and also stimulates the synthesis of uric acid by amino acid precursors. Therefore, inhibiting KHK can effectively inhibit fructose metabolism and the resulting lipid accumulation, oxidative stress, inflammation and insulin resistance, which is beneficial for preventing and treating NASH and gout, as well as many diseases that involve changes in either or both of endogenous or ingested fructose.

### SUMMARY OF THE INVENTION

An object of the present invention is to provide a KHK inhibitor, a preparation method therefor, and application thereof. The series of compounds of the present invention have a strong inhibitory effect on KHK, and can be widely used in the preparation of medicaments for treatment and/or prevention of KHK-related diseases, making it hopeful to develop a new generation of KHK inhibitors.

The first aspect of the present invention provides a compound of formula (I), a stereoisomer or pharmaceutically acceptable salt thereof.

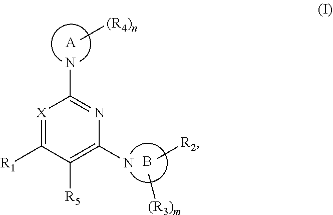

wherein:
Ring A is 4-6 membered nitrogen-containing heteromonocyclyl or 7-12 membered nitrogen-containing heterobicyclyl, and the bicyclyl is a spiro, fused, or bridged ring;
Ring B is 4-6 membered nitrogen-containing heteromonocyclyl or 7-12 membered nitrogen-containing heterobicyclyl, and the bicyclyl is a spiro, fused, or bridged ring;
X is N or CRₐ; Rₐ is selected from the group consisting of hydrogen, deuterium, cyano, trifluoromethyl, and aminoacyl;
R₁ is trifluoromethyl, or, R₁ and Rₐ, together with the moiety directly attached thereto, form structures as follows:

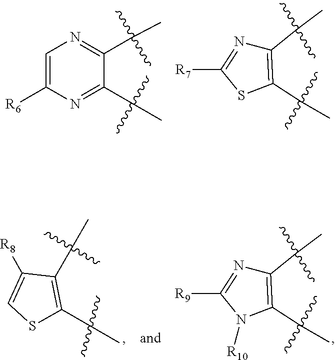

or, R₁ and R₅, together with the moiety directly attached thereto, form a structure as follows:

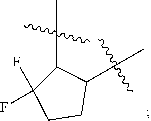 [SMILES: *C1CCC(F)(F)C1*]

R₂ is hydrogen or -L-C(O)—R; L is a bond or C₁₋₆ alkylene, the above C₁₋₆ alkylene is optionally further substituted by a substituent selected from the group consisting of deuterium, halogen, and C₁₋₆ alkyl; R is selected from the group consisting of hydroxy, amino, hydroxyamino, C₁₋₆ alkylamino, C₁₋₆ alkyl, and C₁₋₆ alkoxy, the above C₁₋₆ alkyl and C₁₋₆ alkoxy are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
each R₃ is independently hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl, the above C₁₋₆ alkyl, C₁₋₆ alkoxy, C₁₋₆ alkylamino, and C₁₋₆ alkylacyl are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
R₅ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl, the above C₁₋₆ alkyl, C₁₋₆ alkoxy, C₁₋₆ alkylamino, and C₁₋₆ alkylacyl are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
R₆, R₇, R₈, and R₉ are each independently selected from the group consisting of hydrogen, deuterium, hydroxy, amino, cyano, aminoacyl, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl, the above C₁₋₆ alkyl, C₁₋₆ alkoxy, C₁₋₆ alkylamino, and C₁₋₆ alkylacyl are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
R₁₀ is selected from the group consisting of hydrogen, deuterium, hydroxy, C₁₋₆ alkyl, and C₁₋₆ alkylacyl, the above C₁₋₆ alkyl and C₁₋₆ alkylacyl are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
m is 0, 1, 2, 3, 4, 5, or 6;
n is 0, 1, 2, 3, or 4.

As a preferred embodiment, in the compound of formula (I), the stereoisomer or pharmaceutically acceptable salt thereof, Ring B is selected from structures as follows:

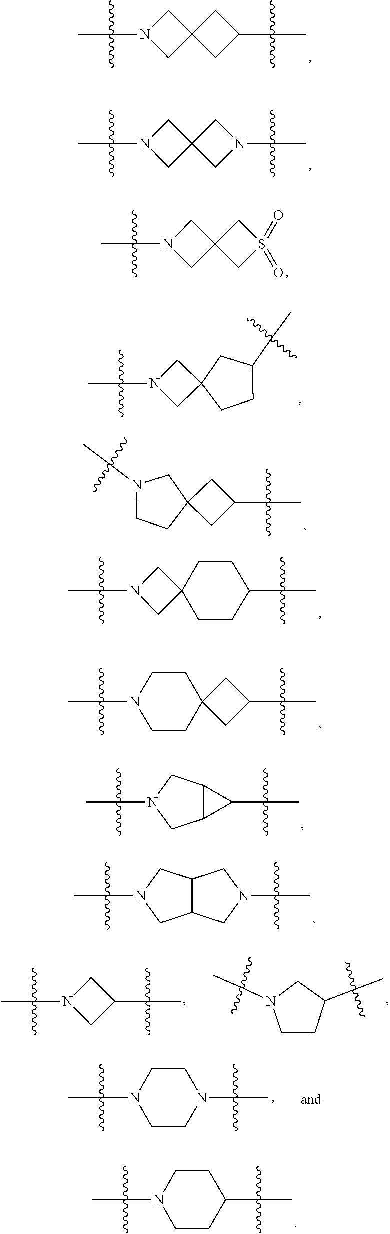

As a further preferred embodiment, in the compound of formula (I), the stereoisomer or pharmaceutically acceptable salt thereof, the compound of formula (I) is a compound with a structure as shown in the following formula (IIa):

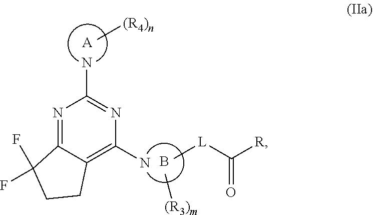

Ring A is 4-6 membered nitrogen-containing heteromonocyclyl, the 4-6 membered nitrogen-containing heteromonocyclyl is selected from the group consisting of azetidine, tetrahydropyrrole, morpholine, piperidine, piperazine, and quinuclidine;

Ring B is selected from the group consisting of

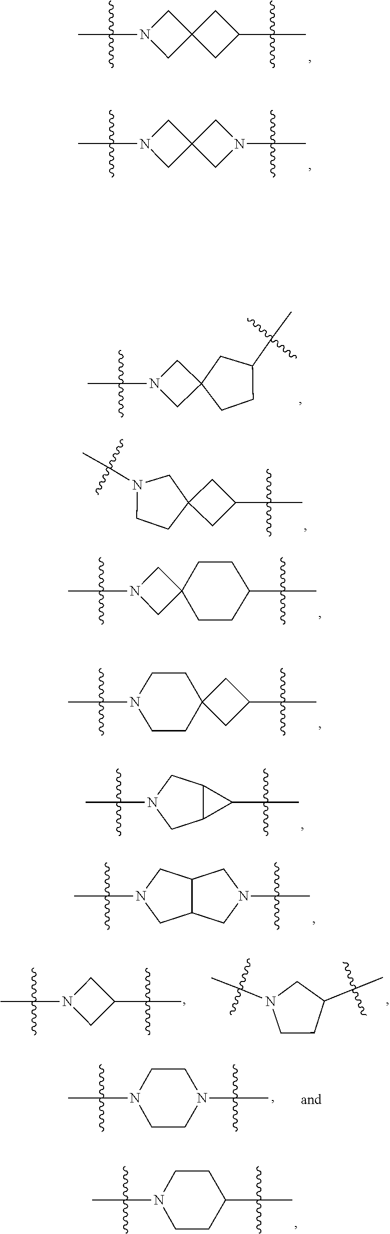

provided that, when Ring B is selected from

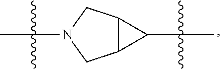 [SMILES: *C1C2CN(*)CC12]

R is hydroxyamino;

L is a bond or C₁₋₃ alkylene, the above C₁₋₃ alkylene is optionally further substituted by a substituent selected from the group consisting of deuterium, halogen, and C₁₋₃ alkyl; R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently selected from hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino; wherein, m and n are as defined in the compound of formula (I).

As a further preferred embodiment, in the compound of formula (I), the stereoisomer or pharmaceutically acceptable salt thereof, the compound of formula (I) is a compound with a structure as shown in the following formula (IIIa):

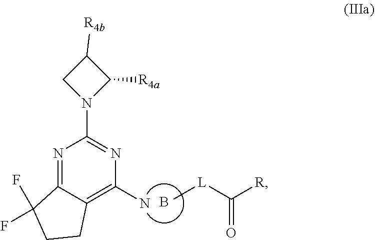

wherein, Ring B is selected from the group consisting of

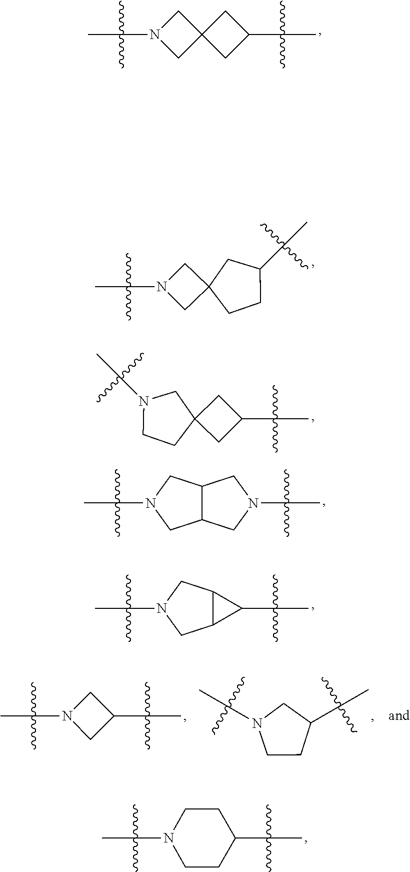

provided that, when Ring B is selected from

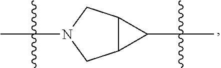 [SMILES: *C1C2CN(*)CC12]

R is hydroxyamino;
L is a bond, methylene, or ethylidene, the above methylene or ethylidene is optionally further substituted by a substituent selected from the group consisting of deuterium, halogen, methyl, ethyl, and isopropyl;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl.

As a further preferred embodiment, in the compound of formula (I), the stereoisomer or pharmaceutically acceptable salt thereof, the compound of formula (I) is a compound with a structure as shown in the following formula (IIb):

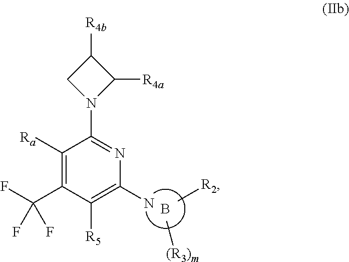

wherein, Rₐ is selected from the group consisting of hydrogen, deuterium, cyano, trifluoromethyl, and aminoacyl;
Ring B is selected from the group consisting of

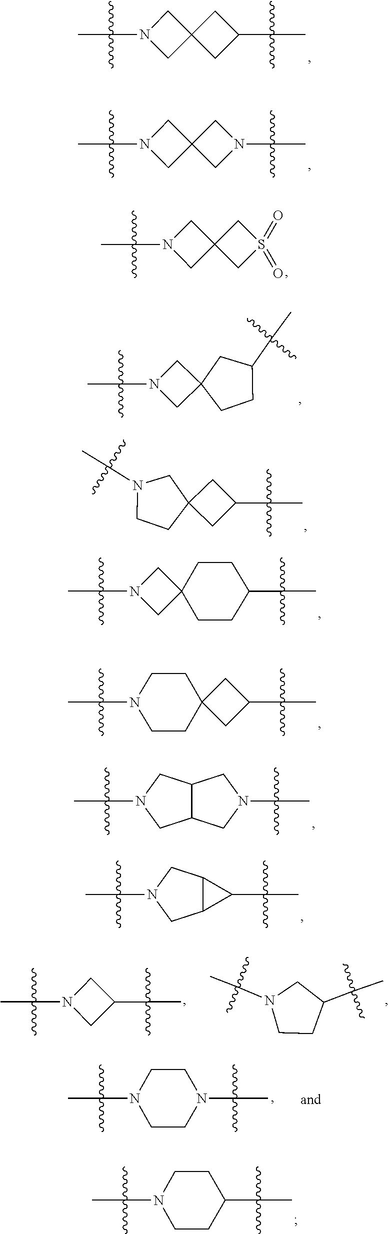

P R₂ is hydrogen or -L-C(O)—R;
L is a bond, methylene, or ethylidene, the above methylene or ethylidene is optionally further substituted by a substituent selected from the group consisting of deuterium, methyl, ethyl, and isopropyl, provided that,
1) when L is unsubstituted methylene, Ring B is not selected from

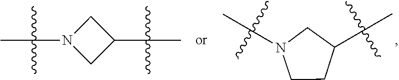 [SMILES: *C1CN(*)C1 ; *C1CCN(*)C1]

2) when L is unsubstituted methylene or unsubstituted ethylidene and Ring B is

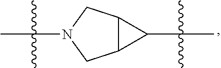 [SMILES: *C1C2CN(*)CC12]

R₄ₐ is methyl or deuteriomethyl and R_{4b} is hydrogen;
R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently hydrogen, deuterium, or fluorine;
R₄ₐ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R_{4b} is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₅ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino; wherein, m is defined as in the compound of formula (I).

As a further preferred embodiment, in the compound of formula (I), the stereoisomer or pharmaceutically acceptable salt thereof, L is a bond, methylene, or ethylidene, the above methylene or ethylidene is optionally further substituted by a substituent selected from the group consisting of deuterium, methyl, ethyl, and isopropyl, provided that,
1) when L is unsubstituted methylene, Ring B is not selected from

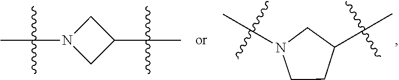 [SMILES: *C1CN(*)C1 ; *C1CCN(*)C1]

2) when L is unsubstituted methylene or unsubstituted ethylidene and Ring B is

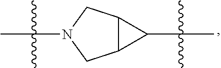 [SMILES: *C1C2CN(*)CC12]

R₄ₐ is methyl or deuteriomethyl and R_{4b} is hydrogen;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl.

As a further preferred embodiment, in the compound of formula (I), the stereoisomer or pharmaceutically acceptable salt thereof, the compound of formula (I) is a compound with a structure as shown in the following formula (IIIb₁):

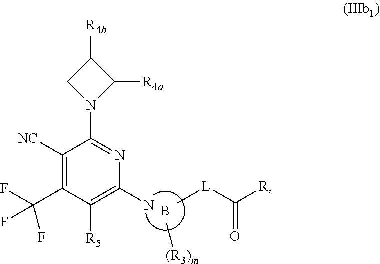

wherein,
Ring B is selected from the group consisting of

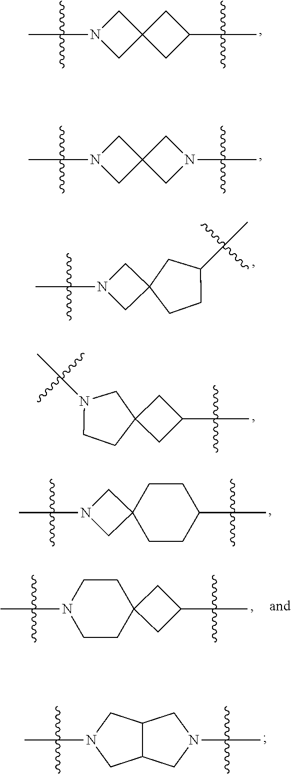

L is a bond, methylene, or ethylidene, the above methylene and ethylidene are optionally further substituted by substituents selected from the group consisting of deuterium, methyl, ethyl, and isopropyl;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
each R₃ is independently hydrogen, deuterium, or fluorine;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from hydrogen and deuterium;
m is 0, 1, 2, 3, 4, 5, or 6.

As a further preferred embodiment, in the compound of formula (I), the stereoisomer or pharmaceutically acceptable salt thereof, the compound of formula (I) is a compound with a structure as shown in the following formula (IIIb₂):

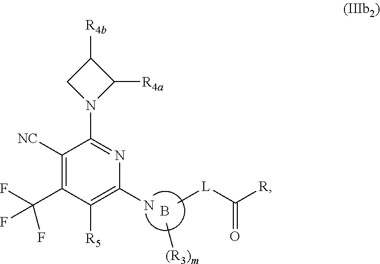

wherein, Ring B is selected from the group consisting of

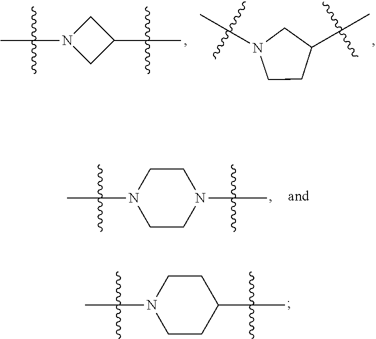 [SMILES: *C1CN(*)C1 ; *C1CCN(*)C1 ; *N1CCN(*)CC1 ; *C1CCN(*)CC1]

L is ethylidene, the above ethylidene is optionally further substituted by a substituent selected from the group consisting of deuterium, methyl, ethyl, and isopropyl;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
each R₃ is independently hydrogen, deuterium, or fluorine;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from hydrogen and deuterium;
m is 0, 1, 2, 3, 4, 5, or 6.

As a further preferred embodiment, in the compound of formula (I), the stereoisomer or pharmaceutically acceptable salt thereof, the compound of formula (I) is a compound with a structure as shown in the following formula (IIIb₃):

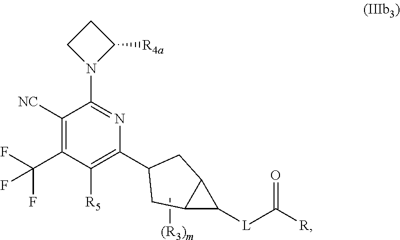

wherein,
L is a bond, methylene, ethylidene, methylomethylene, deuteriomethylene, and deuterioethylidene;
R is selected from the group consisting of hydroxy, amino, hydroxyamino, methoxy, ethoxy, isopropoxy, trifluoromethoxy, and trideuteriomethoxy;
each R₃ is independently hydrogen, deuterium, or fluorine;
R₄ₐ is methyl and trideuteriomethyl;
R₅ is hydrogen and deuterium;
m is 0, 1, 2, 3, 4, 5, or 6.

As a further preferred embodiment, in the compound of formula (I), the stereoisomer or pharmaceutically acceptable salt thereof, L, R₃, R₄ₐ, and R₅ comprise at least one deuterium atom.

As a further preferred embodiment, in the compound of formula (I), the stereoisomer or pharmaceutically acceptable salt thereof, L, R₃, R₄ₐ, and R₅ comprise 1, 2, 3, 4, 5, and 6 deuterium atoms.

As a preferred embodiment, in the compound of formula (I), the stereoisomer or pharmaceutically acceptable salt thereof, the compound of formula (I) is a compound with a structure shown as the following formula (IIc):

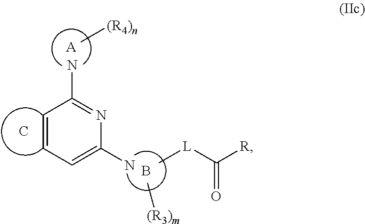

wherein, Ring C is selected from structures as follows:

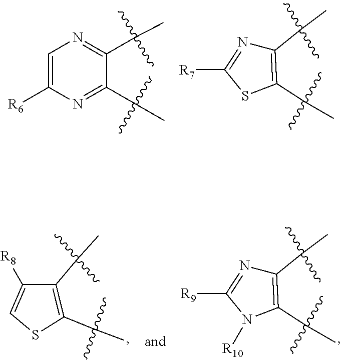

Ring A is 4-6 membered nitrogen-containing heteromonocyclyl, the 4-6 membered nitrogen-containing heteromonocyclyl is selected from the group consisting of azetidine, tetrahydropyrrole, morpholine, piperidine, piperazine, and quinuclidine;
Ring B is selected from structures as follows:

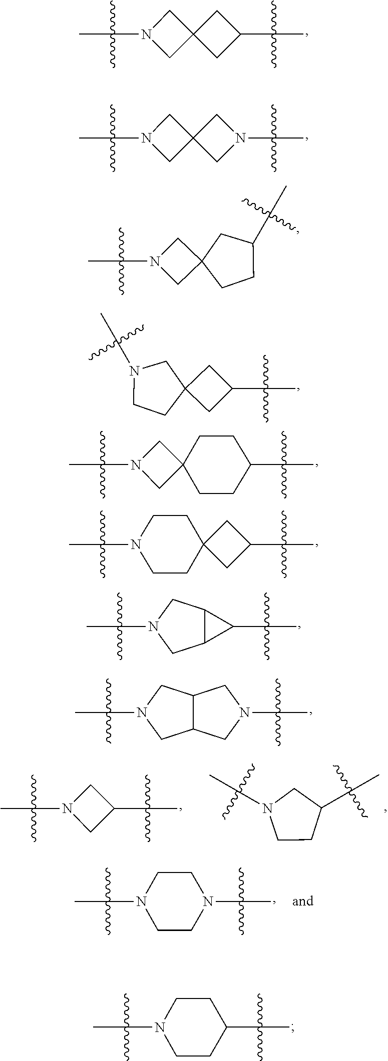

L is a bond or C₁₋₃ alkylene, the above C₁₋₃ alkylene is optionally further substituted by a substituent selected from the group consisting of deuterium, halogen, and C₁₋₃ alkyl;
R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₆, R₇, R₈, and R₉ are each independently selected from the group consisting of hydrogen, deuterium, hydroxy, amino, cyano, aminoacyl, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₁₀ is selected from the group consisting of hydrogen, deuterium, hydroxy, C₁₋₃ alkyl, C₁₋₃ haloalkyl, C₁₋₃ deuterioalkyl, and C₁₋₃ alkylacyl; wherein, m and n are defined as in the compound of formula (I).

As a further preferred embodiment, in the compound of formula (I), the stereoisomer or pharmaceutically acceptable salt thereof, the compound of formula (I) is a compound with a structure as shown in the following formula (IIc):

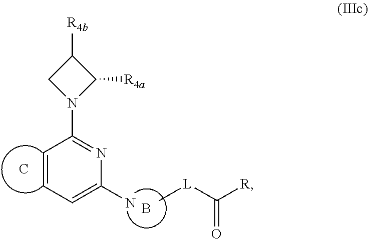

wherein, Ring C is selected from structures as follows:

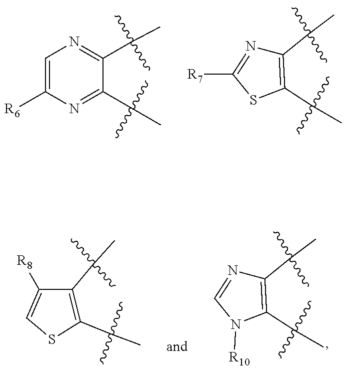

Ring B is

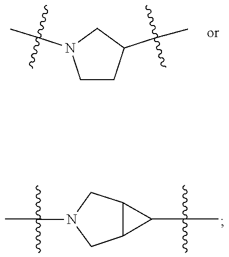 [SMILES: *C1CCN(*)C1 ; *C1C2CN(*)CC12]

L is a bond, methylene, or ethylidene, the above methylene or ethylidene is optionally further substituted by a substituent selected from the group consisting of deuterium, halogen, methyl, ethyl, and isopropyl;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₆, R₇, and R₈ are each independently selected from the group consisting of hydrogen, deuterium, hydroxy, amino, cyano, aminoacyl, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₁₀ is selected from the group consisting of hydrogen, deuterium, hydroxy, methyl, ethyl, isopropyl, trifluoromethyl, trideuteriomethyl, and acetyl.

As the most preferred embodiment, the compound of formula (I), the stereoisomer or pharmaceutically acceptable salt thereof includes, but is not limited to, compounds as follows:

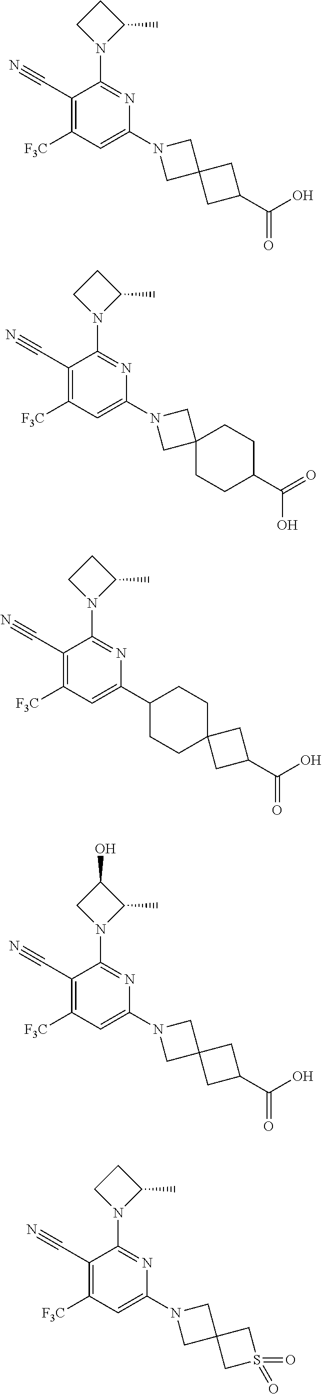 [SMILES: C[C@H]1CCN1c1nc(N2CC3(CC(C(=O)O)C3)C2)cc(C(F)(F)F)c1C#N ; C[C@H]1CCN1c1nc(N2CC3(CCC(C(=O)O)CC3)C2)cc(C(F)(F)F)c1C#N ; C[C@H]1CCN1c1nc(C2CCC3(CC2)CC(C(=O)O)C3)cc(C(F)(F)F)c1C#N ; C[C@H]1[C@H](O)CN1c1nc(N2CC3(CC(C(=O)O)C3)C2)cc(C(F)(F)F)c1C#N ; C[C@H]1CCN1c1nc(N2CC3(C2)CS(=O)(=O)C3)cc(C(F)(F)F)c1C#N]

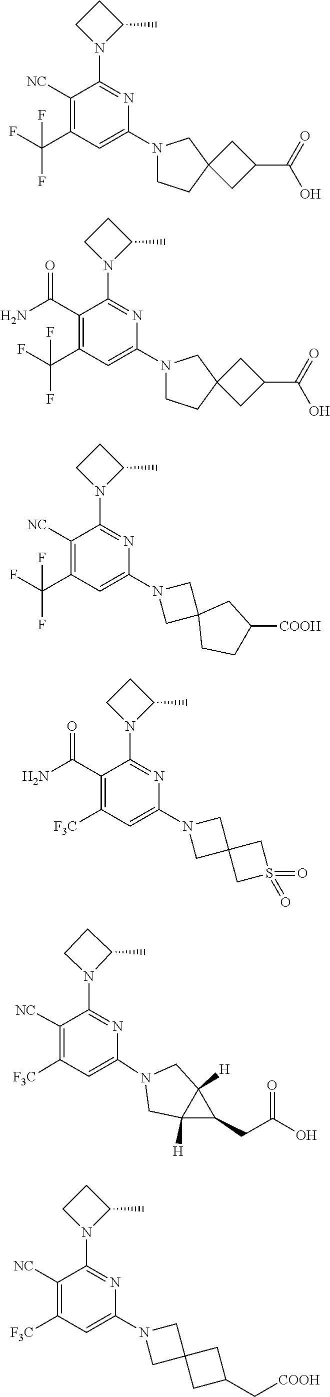 [SMILES: C[C@H]1CCN1c1nc(N2CCC3(CC(C(=O)O)C3)C2)cc(C(F)(F)F)c1C#N ; C[C@H]1CCN1c1nc(N2CCC3(CC(C(=O)O)C3)C2)cc(C(F)(F)F)c1C(N)=O ; C[C@H]1CCN1c1nc(N2CC3(CCC(C(=O)O)C3)C2)cc(C(F)(F)F)c1C#N ; C[C@H]1CCN1c1nc(N2CC3(C2)CS(=O)(=O)C3)cc(C(F)(F)F)c1C(N)=O ; C[C@H]1CCN1c1nc(N2C[C@@H]3[C@@H](CC(=O)O)[C@@H]3C2)cc(C(F)(F)F)c1C#N ; C[C@H]1CCN1c1nc(N2CC3(CC(CC(=O)O)C3)C2)cc(C(F)(F)F)c1C#N]

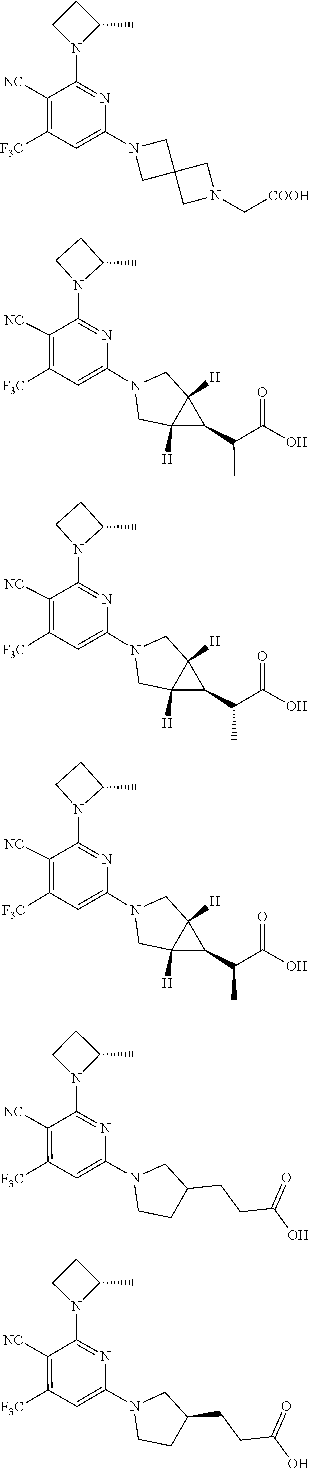 [SMILES: C[C@H]1CCN1c1nc(N2CC3(CN(CC(=O)O)C3)C2)cc(C(F)(F)F)c1C#N ; CC(C(=O)O)[C@H]1[C@@H]2CN(c3cc(C(F)(F)F)c(C#N)c(N4CC[C@@H]4C)n3)C[C@@H]21 ; C[C@@H](C(=O)O)[C@H]1[C@@H]2CN(c3cc(C(F)(F)F)c(C#N)c(N4CC[C@@H]4C)n3)C[C@@H]21 ; C[C@H](C(=O)O)[C@H]1[C@@H]2CN(c3cc(C(F)(F)F)c(C#N)c(N4CC[C@@H]4C)n3)C[C@@H]21 ; C[C@H]1CCN1c1nc(N2CCC(CCC(=O)O)C2)cc(C(F)(F)F)c1C#N ; C[C@H]1CCN1c1nc(N2CC[C@H](CCC(=O)O)C2)cc(C(F)(F)F)c1C#N]

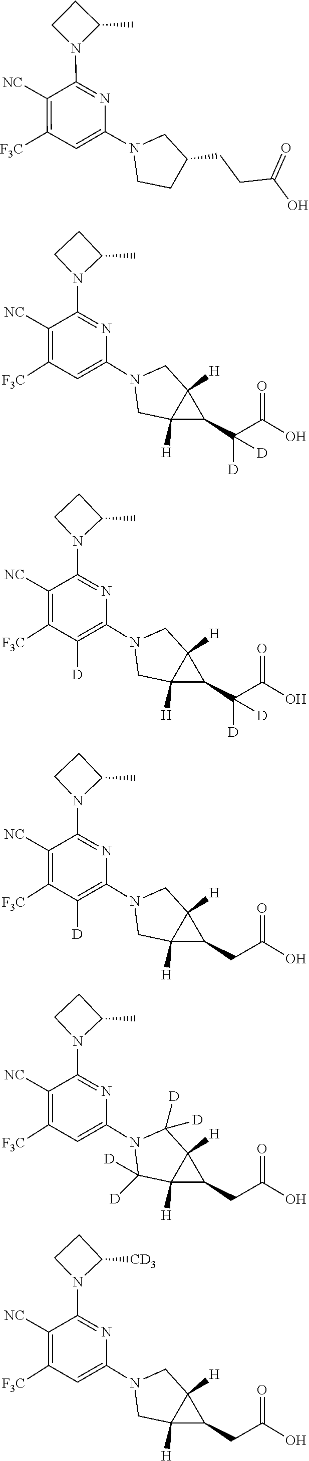 [SMILES: C[C@H]1CCN1c1nc(N2CC[C@@H](CCC(=O)O)C2)cc(C(F)(F)F)c1C#N ; [2H]C([2H])(C(=O)O)[C@H]1[C@@H]2CN(c3cc(C(F)(F)F)c(C#N)c(N4CC[C@@H]4C)n3)C[C@@H]21 ; [2H]c1c(N2C[C@@H]3[C@H](C2)[C@H]3C([2H])([2H])C(=O)O)nc(N2CC[C@@H]2C)c(C#N)c1C(F)(F)F ; [2H]c1c(N2C[C@@H]3[C@@H](CC(=O)O)[C@@H]3C2)nc(N2CC[C@@H]2C)c(C#N)c1C(F)(F)F ; [2H]C1([2H])[C@@H]2[C@@H](CC(=O)O)[C@@H]2C([2H])([2H])N1c1cc(C(F)(F)F)c(C#N)c(N2CC[C@@H]2C)n1 ; [2H]C([2H])([2H])[C@H]1CCN1c1nc(N2C[C@@H]3[C@@H](CC(=O)O)[C@@H]3C2)cc(C(F)(F)F)c1C#N]

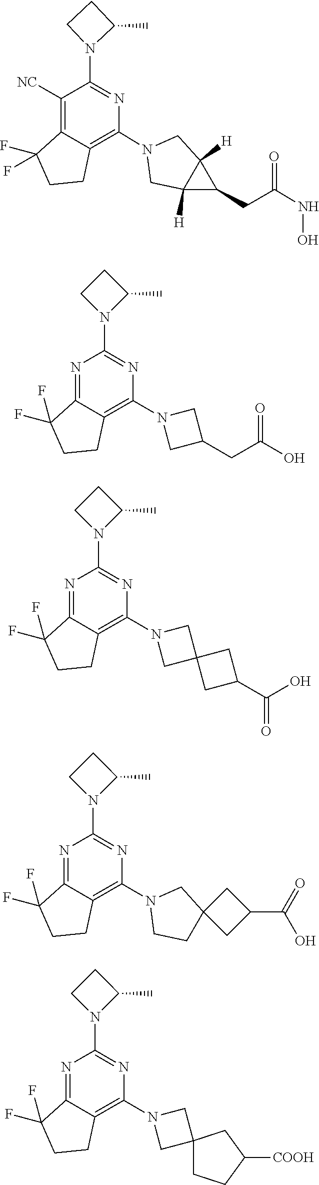

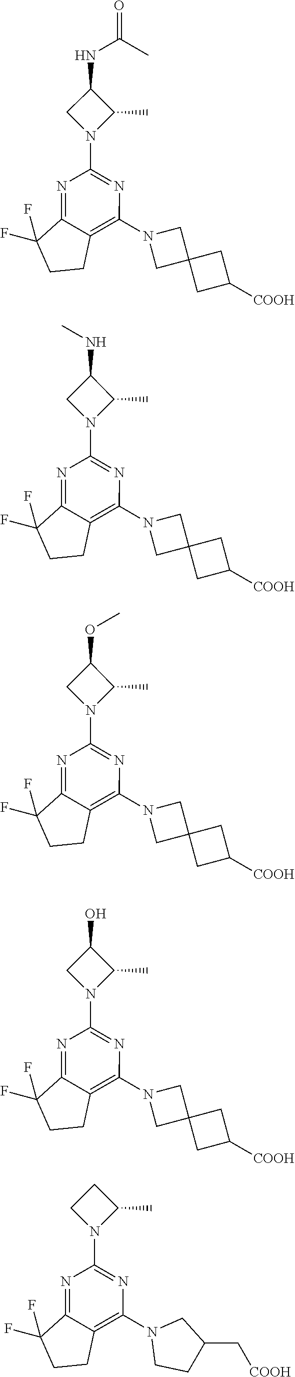 [SMILES: CC(=O)N[C@@H]1CN(c2nc(N3CC4(CC(C(=O)O)C4)C3)c3c(n2)C(F)(F)CC3)[C@H]1C ; CN[C@@H]1CN(c2nc(N3CC4(CC(C(=O)O)C4)C3)c3c(n2)C(F)(F)CC3)[C@H]1C ; CO[C@@H]1CN(c2nc(N3CC4(CC(C(=O)O)C4)C3)c3c(n2)C(F)(F)CC3)[C@H]1C ; C[C@H]1[C@H](O)CN1c1nc(N2CC3(CC(C(=O)O)C3)C2)c2c(n1)C(F)(F)CC2 ; C[C@H]1CCN1c1nc(N2CCC(CC(=O)O)C2)c2c(n1)C(F)(F)CC2]

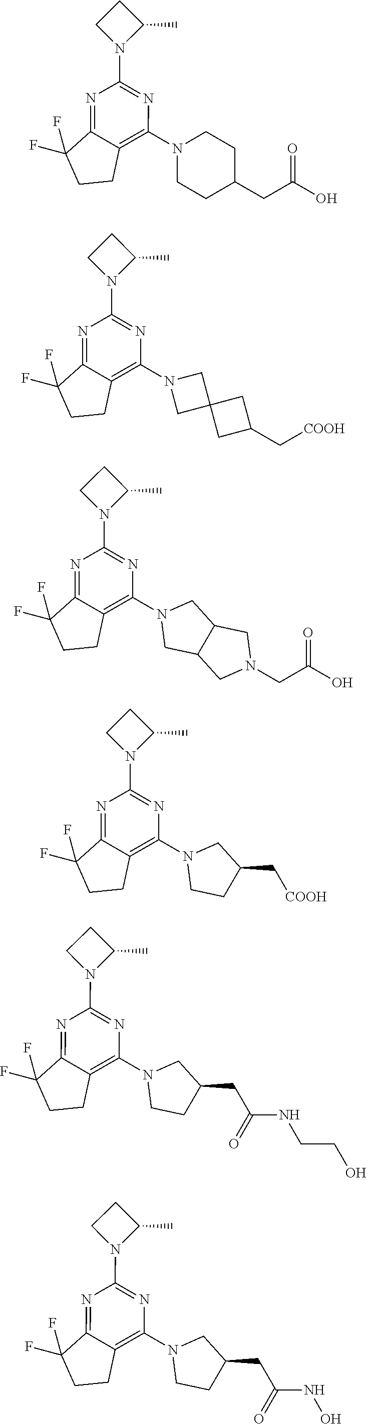

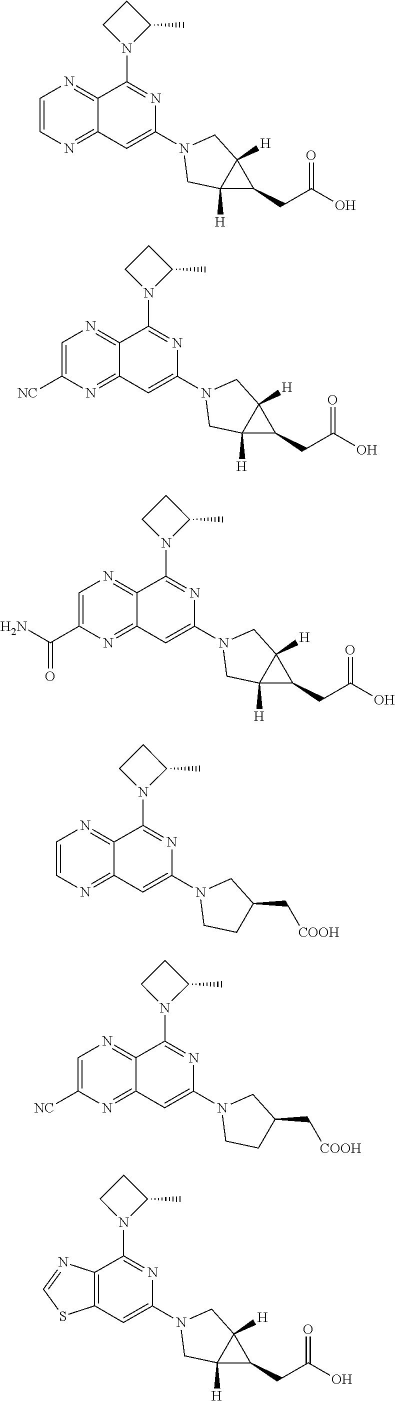 [SMILES: C[C@H]1CCN1c1nc(N2C[C@@H]3[C@@H](CC(=O)O)[C@@H]3C2)cc2nccnc12 ; C[C@H]1CCN1c1nc(N2C[C@@H]3[C@@H](CC(=O)O)[C@@H]3C2)cc2nc(C#N)cnc12 ; C[C@H]1CCN1c1nc(N2C[C@@H]3[C@@H](CC(=O)O)[C@@H]3C2)cc2nc(C(N)=O)cnc12 ; C[C@H]1CCN1c1nc(N2CC[C@H](CC(=O)O)C2)cc2nccnc12 ; C[C@H]1CCN1c1nc(N2CC[C@H](CC(=O)O)C2)cc2nc(C#N)cnc12 ; C[C@H]1CCN1c1nc(N2C[C@@H]3[C@@H](CC(=O)O)[C@@H]3C2)cc2scnc12]

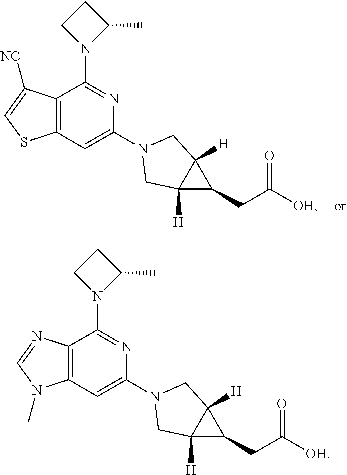 [SMILES: C[C@H]1CCN1c1nc(N2C[C@@H]3[C@@H](CC(=O)O)[C@@H]3C2)cc2scc(C#N)c12 ; C[C@H]1CCN1c1nc(N2C[C@@H]3[C@@H](CC(=O)O)[C@@H]3C2)cc2c1ncn2C]

The second aspect of the present invention provides a pharmaceutical composition, comprising the compound of formula (I), the stereoisomer or pharmaceutically acceptable salt thereof, and one or more pharmaceutically acceptable carriers.

The third aspect of the present invention provides a use of the compound of formula (I), the stereoisomer or pharmaceutically acceptable salt thereof in preparation of a medicament for treatment and/or prevention of KHK-mediated diseases.

Preferably, the KHK-mediated diseases are selected from the group consisting of endocrine disorders, urological disorders, metabolic disorders, non-alcoholic steatohepatitis, cirrhosis, fatty liver, hepatitis, liver failure, hereditary fructose intolerance, non-alcoholic fatty liver disease, hepatobiliary disorders, fibrotic disorders, cardiovascular and cerebrovascular disorders, immunoinflammatory disorders, central nervous system disorders, gastrointestinal disorders, and hyperproliferative disorders (e.g., cancer).

### DETAILED DESCRIPTION OF THE INVENTION

Based on extensive and in-depth studies, the inventors of the present invention developed a KHK inhibitor with a structure of formula (I) below for the first time. The series of compounds of the present invention can be widely used in the preparation of medicaments for treatment and/or prevention of KHK-related diseases, making it hopeful to develop a new generation of KHK inhibitors. The present invention is achieved on this basis.

Detailed description: Unless otherwise stated to the contrary or specifically noted, the following terms used in the specification and claims have the meanings described below.

“Alkyl” refers to linear or branched saturated aliphatic hydrocarbon groups, preferably linear or branched alkyl containing 1 to 6 carbon atoms or 1 to 3 carbon atoms, including but not limited to: methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl or various branched isomers thereof, etc. “C₁₋₆ alkyl” refers to linear and branched alkyl including 1-6 carbon atoms, and “C₁₋₃ alkyl” refers to linear and branched alkyl including 1-3 carbon atoms.

Alkyl may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Cycloalkyl” or “carbocycle” refers to a monocyclic or polycyclic hydrocarbon substituent that is saturated or partially unsaturated. The partially unsaturated cyclic hydrocarbon means that the cyclic hydrocarbon may contain one or more (preferably, 1, 2, or 3) double bonds, but no ring has a fully conjugated 7-electron system. The cycloalkyl includes monocyclic cycloalkyl and polycyclic cycloalkyl, preferably including a cycloalkyl containing 3 to 12 or 3 to 8 or 3 to 6 carbon atoms. For example, “C₃₋₁₂ cycloalkyl” means a cycloalkyl containing 3 to 12 carbon atoms, “C₃₋₁₀ cycloalkyl” means a cycloalkyl containing 3 to 10 carbon atoms, “C₃₋₈ cycloalkyl” means a cycloalkyl containing 3 to 8 carbon atoms, and “C₃₋₆ cycloalkyl” means a cycloalkyl containing 3 to 6 carbon atoms, wherein:
monocyclic cycloalkyl includes, but is not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl and the like.
polycyclic cycloalkyl includes spiro, fused, and bridged cycloalkyl. “Spirocycloalkyl” refers to a polycyclic group in which a carbon atom (called spiro-atom) is shared among monocyclic rings, wherein those rings may contain one or more (preferably, 1, 2, or 3) double bonds, but no ring has a fully conjugated π-electron system. According to the number of the spiro-atoms shared among the rings, the spirocycloalkyl may be monospirocycloalkyl, bispirocycloalkyl or polyspirocycloalkyl, including but not limited to:

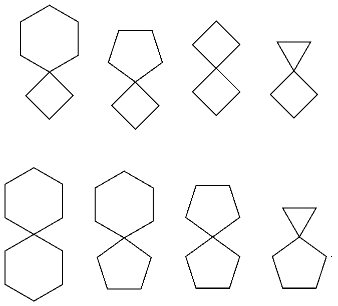

“Fused cycloalkyl” refers to an all-carbon polycyclic group in which each ring shares a pair of adjacent carbon atoms with the other rings in the system, wherein one or more of the rings may contain one or more (preferably, 1, 2, or 3) double bonds, but no ring has a fully conjugated π-electron system. According to the number of formed rings, the fused cycloalkyl may be bicyclic, tricyclic, tetracyclic or polycyclic, including but not limited to:

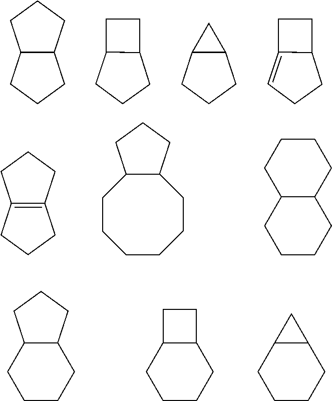

“Bridged cycloalkyl” refers to an all-carbon polycyclic group in which any two rings share two carbon atoms that are not directly connected to each other, wherein these rings may contain one or more (preferably, 1, 2, or 3) double bonds, but no ring has a fully conjugated π-electron system. According to the number of formed rings, the bridged cycloalkyl may be bicyclic, tricyclic, tetracyclic or polycyclic, including but not limited to:

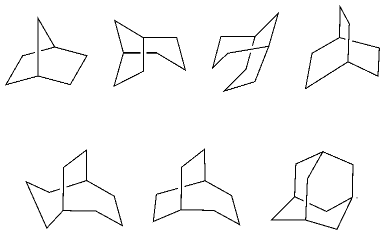

The cycloalkyl ring can be fused to an aryl, heteroaryl or heterocycloalkyl ring, wherein the ring attached to the parent structure is cycloalkyl, which includes, but is not limited to, indanyl, tetrahydronaphthyl, benzocycloheptyl, etc.

The cycloalkyl may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Heterocyclyl” or “heterocycle” refers to a saturated or partially unsaturated monocyclic or polycyclic hydrocarbon substituent. The partially unsaturated cyclic hydrocarbon means that the cyclic hydrocarbon may contain one or more (preferably, 1, 2, or 3) double bonds, but no ring has a fully conjugated π-electron system, wherein one or more (preferably, 1, 2, 3 or 4) of the ring atoms are heteroatoms selected from N, O, NO, or S(O)ᵣ (wherein r is an integer of 0, 1 or 2), but excluding ring moiety of —O—O—, —O—S— or —S—S—, and the remaining ring atoms are carbon atoms. Heterocyclyl including 3 to 12 or 3 to 8 or 3 to 6 ring atoms is preferred. For example, “3-6 membered heterocyclyl” refers to heterocyclyl containing 3 to 6 ring atoms, “3-8 membered heterocyclyl” refers to heterocyclyl containing 3 to 8 ring atoms, “4-8 membered heterocyclyl” refers to heterocyclyl containing 4 to 8 ring atoms, “4-10 membered heterocyclyl” refers to heterocyclyl containing 4 to 10 ring atoms, “5-8 membered heterocyclyl” refers to heterocyclyl containing 5 to 8 ring atoms, and “3-12 membered heterocyclyl” refers to heterocyclyl containing 3 to 12 ring atoms.

Monocyclic heterocyclyl includes, but is not limited to, pyrrolidinyl, piperidinyl, piperazinyl, morpholinyl, thiomorpholinyl, homopiperazinyl, oxetane, tetrahydrofuran and the like.

Polycyclic heterocyclyl includes spiro, fused, and bridged heterocyclyl. “Spiroheterocyclyl” refers to polycyclic heterocyclyl that shares a carbon atom (called a spiro atom) between the monocyclic rings, wherein one or more (preferably, 1, 2, 3 or 4) ring atoms are heteroatoms selected from N, O, NO, or S(O)ᵣ (wherein r is an integer of 0, 1 or 2), and the remaining ring atoms are carbon atoms. These groups may contain one or more (preferably, 1, 2, or 3) double bonds, but no ring has a fully conjugated π-electron system. The spiroheterocyclyl may be a monospiroheterocyclyl, a bispiroheterocyclyl or a polyspiroheterocyclyl according to the number of spiro atoms shared between the rings. Spiroheterocyclyl includes, but is not limited to:

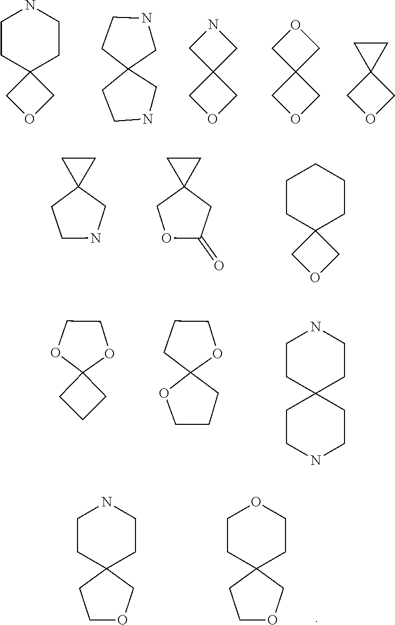

“Fused heterocyclyl” refers to polycyclic heterocyclyl in which each ring shares an adjacent pair of carbon atoms with other rings in the system, wherein one or more (preferably, 1, 2, 3 or 4) of the rings may contain one or more (preferably, 1, 2 or 3) double bonds, but no ring has a fully conjugated π-electron system, wherein one or more (preferably, 1, 2, 3 or 4) of the ring atoms are heteroatoms selected from N, O, NO, or S(O)ᵣ (wherein r is an integer of 0, 1, 2), and the remaining ring atoms are carbon atoms. Depending on the number of rings, it may be bicyclic, tricyclic, tetracyclic or polycyclic, and the fused heterocyclyl includes, but is not limited to:

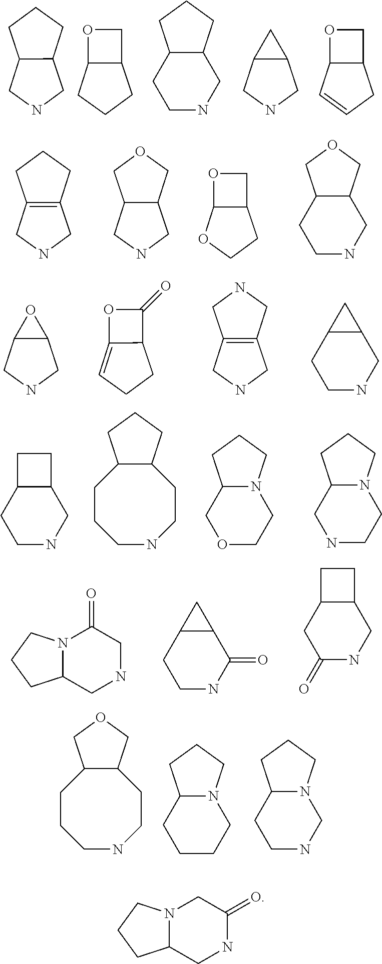

“Bridged heterocyclyl” refers to polycyclic heterocyclyl in which any two rings share two carbon atoms that are not directly bonded, which may contain one or more (preferably, 1, 2 or 3) double bonds, but no ring has a fully conjugated pi-electron system, wherein one or more (preferably, 1, 2, 3 or 4) of the ring atoms are heteroatoms selected from N, O, NO, or S(O)ᵣ (wherein r is an integer of 0, 1, 2), and the remaining ring atoms are carbon atoms. Depending on the number of rings, it may be bicyclic, tricyclic, tetracyclic or polycyclic, and bridged heterocyclyl includes, but is not limited to:

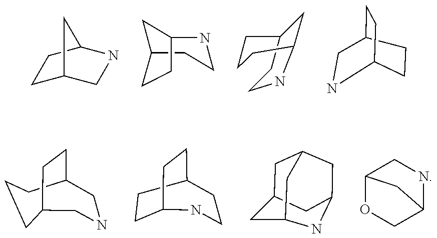

The ring of the heterocyclyl may be fused to a ring of aryl, heteroaryl or cycloalkyl wherein the ring attached to the parent structure is a heterocyclyl, includes, but is not limited to:

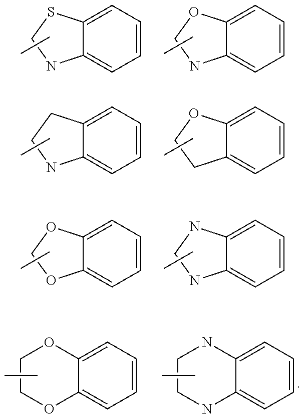

The heterocyclyl may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Aryl” or “aromatic ring” refers to an all-carbon monocyclic or fused polycyclic (i.e., a ring that shares a pair of adjacent carbon atoms) group, and a polycyclic group having a conjugated π-electron system (i.e., a ring with adjacent pairs of carbon atoms). The all-carbon aryl containing 6-10 or 6-8 carbons is preferred. For example, “C₆₋₁₀ aryl” refers to all-carbon aryl containing 6 to 10 carbons, including but not limited to phenyl and naphthyl; and “C₆₋₈ aryl” refers to all-carbon aryl containing 6 to 8 carbons, including but not limited to phenyl and naphthyl. An aryl ring may be fused to a ring of heteroaryl, heterocyclyl or cycloalkyl, wherein the ring attached to the parent structure is an aryl ring, including, but not limited to:

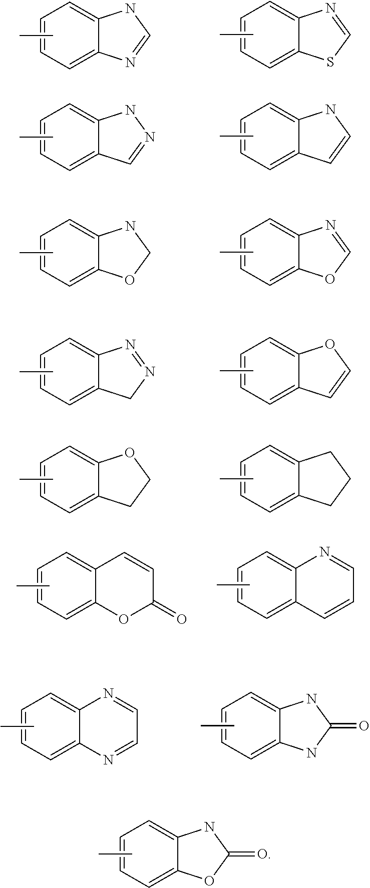

“Aryl” may be substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Heteroaryl” refers to a heteroaromatic system containing one or more (preferably, 1, 2, 3 or 4) heteroatoms including a heteroatom selected from the group consisting of N, O, N atom, and S(O)ᵣ (wherein r is an integer of 0, 1, 2). The heteroaromatic system containing 5-10 or 5-8 or 5-6 ring atoms is preferred. For example, “5-8 membered heteroaryl” refers to a heteroaromatic system containing 5 to 8 ring atoms, “5-10 membered heteroaryl” refers to a heteroaromatic system containing 5 to 10 ring atoms, including but not limited to furyl, thiophenyl, pyridyl, pyrrolyl, N-alkylpyrrolyl, pyrimidinyl, pyrazinyl, imidazolyl, tetrazolyl group or the like. The heteroaryl ring may be fused to a ring of aryl, heterocyclyl or cycloalkyl wherein the ring attached to the parent structure is a heteroaryl ring, includes, but is not limited to:

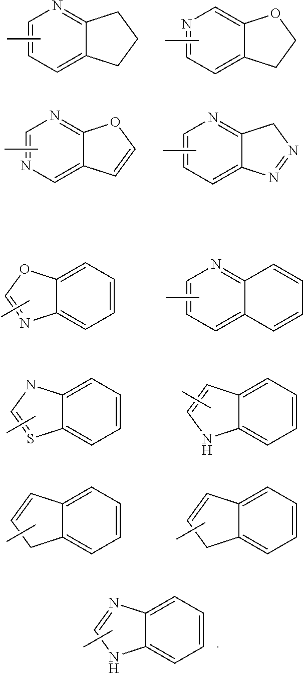

“Heteroaryl” may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Alkenyl” refers to alkyl as defined above consisting of at least two carbon atoms and at least one carbon-carbon double bond. Linear or branched alkenyl containing 2-10 or 2-4 carbons are preferred. For example, “C₂₋₁₀ alkenyl” refers to linear or branched alkenyl containing 2 to 10 carbons, and “C₂₋₄ alkenyl” refers to linear or branched alkenyl containing 2 to 4 carbons. Alkenyl includes, but is not limited to, vinyl, 1-propenyl, 2-propenyl, 1-, 2- or 3-butenyl, and the like.

“Alkenyl” may be substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Alkynyl” refers to an alkyl group defined as above consisting of at least two carbon atoms and at least one carbon-carbon double bond. Linear or branched alkynyl containing 2-10 or 2-4 carbons is preferred. For example, “C₂₋₁₀ alkynyl” refers to linear or branched alkynyl containing 2 to 10 carbons, and “C₂₋₄ alkynyl” refers to linear or branched alkynyl containing 2 to 4 carbons. Alkynyl includes, but is not limited to, ethynyl, 1-propynyl, 2-propynyl, 1-, 2- or 3-butynyl, and the like.

“Alkynyl” may be substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Alkoxy” refers to —O-alkyl, wherein alkyl is defined as above. For example, “C₁₋₁₀ alkoxy” refers to alkyloxy containing 1 to 10 carbons, “C₁₋₄ alkoxy” refers to alkyloxy containing 1-4 carbons, and “C₁₋₂ alkoxy” refers to alkyloxy containing 1 to 2 carbons. The alkoxy includes, but is not limited to, methoxy, ethoxy, propoxy, butoxy, and the like.

“Alkoxy” may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Cycloalkoxy” or “cycloalkyloxy” refers to —O-cycloalkyl, wherein the cycloalkyl is as defined above. For example, “C₃₋₁₂ cycloalkyloxy” refers to cycloalkyloxy containing 3 to 12 carbon atoms, and “C₃₋₆ cycloalkoxy” refers to cycloalkyloxy containing 3 to 6 carbons. The cycloalkyloxy includes, but is not limited to, cyclopropyloxy, cyclobutyloxy, cyclopentyloxy, cyclohexyloxy and the like.

“Cycloalkoxy” or “cycloalkyloxy” may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“Heterocycloxy” or “Heterocyclyloxy” refers to —O-heterocyclyl, wherein the heterocyclyl is as defined above. The heterocyclyloxy includes, but is not limited to, azacyclobutyloxy, oxacyclobutyloxy, azacyclopentyloxy, nitrogen, oxacyclohexyloxy, etc.

“Heterocycloxy” or “heterocyclyloxy” may be optionally substituted or unsubstituted, and when it is substituted, the substituent is preferably one or more (preferably, 1, 2, 3 or 4) groups independently selected from the group consisting of deuterium, halogen, cyano, nitro, azido, C₁₋₁₀ alkyl, C₁₋₁₀ haloalkyl, C₁₋₁₀ deuterioalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, and —C₀₋₈ alkyl-SF₅.

“C₁₋₁₀ haloalkoxy” refers to an alkoxy group having 1 to 10 carbon atoms, in which the hydrogen atoms on the alkyl group are optionally substituted with F, Cl, Br or I atoms. It includes, but is not limited to, difluoromethoxy, dichloromethoxy, dibromomethoxy, trifluoromethoxy, trichloromethoxy, tribromomethoxy, and the like.

“C₁₋₁₀ deuterioalkyl” refers to an alkyl group having 1 to 10 carbon atoms, in which the hydrogen atoms on the alkyl group are optionally substituted with deuterium atoms. It includes, but is not limited to, monodeuterioethoxy, dideuteriomethoxy, trideuteriomethoxy, and the like.

“Halogen” refers to F, Cl, Br or I. “THF” refers to tetrahydrofuran. “PE” refers to petroleum ether; “EtOAc” or “EA” refers to ethyl acetate; “DMF” refers to dimethylformamide; “mCPBA” refers to metachloroperbenzoic acid; “DIPEA” refers to N,N-diisopropylethylamine, “NMP” refers to N-methyl pyrrolidone; “HATU” refers to 2-(7-azobenzotriazole)-N,N,N′,N′-tetramethyluronium hexafluorophosphate (CAS #148893-10-1); and “NBS” refers to N-bromosuccinimide. “Pd₂(dba)₃” refers to tris(dibenzylideneacetone)dipalladium; “XantPhos” refers to 4,5-bis(diphenylphosphino)-9,9-dimethylxanthene; “Cs₂CO₃” refers to cesium carbonate; and “THF” refers to tetrahydrofuran.

“Optional” or “optionally” means that the event or environment subsequently described may, but need not, occur, including where the event or environment occurs or does not occur, that is, including both substituted and unsubstituted situations. For example, “heterocyclyl optionally substituted by alkyl” means that an alkyl group may be, but is not necessarily, present, and the description includes cases where heterocyclyl is substituted with alkyl and cases where heterocyclyl is not substituted with alkyl.

The term “substituted” means that one or more “hydrogen atoms” in the group are each independently substituted by a corresponding number of substituents. It goes without saying that a substituent is only in its possible chemical position, which is consistent with the valence-bond theory of chemistry. Those skilled in the art will be able to determine (by experiments or theories) possible or impossible substitution without undue efforts. For example, it may be unstable when an amino or hydroxy having a free hydrogen is bonded to a carbon atom having an unsaturated bond (such as olefin).

“Stereoisomer” refers to an isomer produced due to a different spatial arrangement of atoms in the molecules, and can be classified into either cis-trans isomers and enantiomers, or enantiomers and diastereomers. Stereoisomers resulting from the rotation of a single bond are called conformational stereo-isomers, and sometimes also called rotamers. Stereoisomers induced by reasons such as bond lengths, bond angles, double bonds in molecules and rings are called configuration stereo-isomers, which are classified into two categories. Among them, isomers induced by the double bonds or single bonds of ring-forming carbon atoms that cannot rotate freely are called geometric isomers, also known as cis-trans isomers, which are classified into two configurations including Z and E. For example: cis-2-butene and trans-2-butene are a pair of geometric isomers. Stereoisomers with different optical activities due to the absence of anti-axial symmetry in the molecules are called optical isomers, which are classified into two configurations including R and S. Unless otherwise specified, the “stereoisomer” in the present invention can be understood to include one or several of the above-mentioned enantiomers, configurational isomers and conformational isomers.

“Pharmaceutically acceptable salt” in the present invention refers to pharmaceutically acceptable acid addition salts, including inorganic acid salts and organic acid salts, and these salts can be prepared by methods known in the art.

“Pharmaceutical composition” refers to a mixture comprising one or more of the compounds described herein, or a physiologically/pharmaceutically acceptable salt or pro-drug thereof, and other chemical components, for example physiological/pharmaceutically acceptable carriers and excipients. The purpose of the pharmaceutical composition is to promote the administration to an organism, which facilitates the absorption of the active ingredient thereby exerting biological activities.

The present invention will be further described in detail below in conjunction with the embodiments, which is not intended to limit the present invention. The present invention is also not limited to the contents of the embodiments.

The structure of the compound of the present invention is determined by nuclear magnetic resonance (NMR) or/and liquid chromatography-mass spectrometry (LC-MS).

The NMR measurement was conducted by a Bruker AVANCE-400 nuclear magnetic apparatus, and the solvent for measurement was deuterated dimethyl sulfoxide (DMSO-d₆), deuterated methanol (CD₃OD) and deuterated chloroform (CDCl₃), and the internal standard is tetramethylsilane (TMS).

The measurement of LC-MS and HPLC was performed by using an Agilent 1200 HPLC/6100 SQ System spectrometer.

The thin layer chromatography silica gel plate is a Yantai Huanghai HSGF254 or Qingdao GF254 silica gel plate, which is 0.15 mm-0.20 mm for TLC and 0.4 mm-0.5 mm for thin layer chromatographic separation and purification of products. 200-300 mesh silica gel (Yantai Huanghai silica gel) is generally used as a carrier in column chromatography.

The starting materials in the examples of the present invention are known and commercially available or can be synthesized according to methods known in the art.

Unless otherwise stated, all reactions of the present invention were conducted under continuous magnetic stirring in the presence of a dry nitrogen or argon atmosphere, the solvent is a dry solvent, and the unit of reaction temperature is Celsius degree (° C.).

### I. PREPARATION OF INTERMEDIATES

### Intermediate A1: Preparation of ethyl 2-((1R,5S,6s)-3-azabicyclo[3.1.0]hexan-6-yl)acetic acid hydrochloride

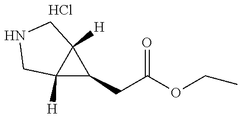

### Step 1: Synthesis of tert-butyl (1R,5S,6r)-6-(bromomethyl)-3-azabicyclo[3.1.0]hexan-3-carboxylate

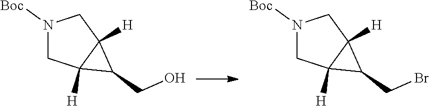 [SMILES: CC(C)(C)OC(=O)N1C[C@@H]2[C@@H](CO)[C@@H]2C1 ; CC(C)(C)OC(=O)N1C[C@@H]2[C@@H](CBr)[C@@H]2C1]

Tert-butyl (1R,5S,6r)-6-(hydroxymethyl)-3-azabicyclo[3.1.0]hexan-3-carboxylate (1.5 g, 7 mmol) and triphenyl phosphine (2.77 g, 10.55 mmol) were dissolved in 100 ml of dichloromethane, stirred, and cooled to 0° C.; NBS (2.77 g, 10.55 mmol) was added; and the resulting mixture was stirred for 4 hrs. The mixture was concentrated directly; and the resulting crude product was separated by column chromatography (PE/EA=10/10) to obtain tert-butyl (1R,5S,6r)-6-(bromomethyl)-3-azabicyclo[3.1.0]hexan-3-carboxylate (1.78 g, yield: 91.6%), LC-MS: m/z=222 (M−56+H)⁺.

### Step 2: Synthesis of tert-butyl (1R,5S,6s)-6-(cyanomethyl)-3-azabicyclo[3.1.0]hexan-3-carboxylate

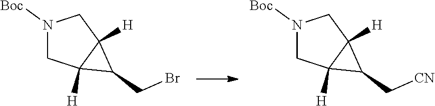 [SMILES: CC(C)(C)OC(=O)N1C[C@@H]2[C@@H](CBr)[C@@H]2C1 ; CC(C)(C)OC(=O)N1C[C@@H]2[C@@H](CC#N)[C@@H]2C1]

Tert-butyl (1R,5S,6r)-6-(bromomethyl)-3-azabicyclo[3.1.0]hexan-3-carboxylate (1.78 g, 6.45 mmol) was dissolved in DMF (25 mL); sodium hydride (0.694 g, 14.18 mmol) was added; and the reaction mixture was stirred for 12 hrs, added with water for quenching, extracted with EA (100 mL*3), washed with water (50 mL*3), dried over anhydrous magnesium sulfate, and concentrated to obtain tert-butyl (1R,5S,6s)-6-(cyanomethyl)-3-azabicyclo[3.1.0]hexan-3-carboxylate (1.3 g, yield: 91%), a yellow liquid. LC-MS: m/z=168.0 (M-56+H)⁺.

### Step 3: Synthesis of ethyl 2-((1R,5S,6s)-3-azabicyclo[3.1.0]hexan-6-yl)acetate hydrochloride

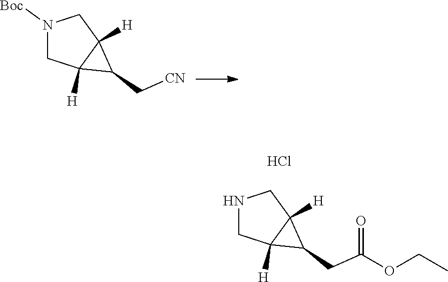 [SMILES: CC(C)(C)OC(=O)N1C[C@@H]2[C@@H](CC#N)[C@@H]2C1 ; Cl ; CCOC(=O)C[C@H]1[C@@H]2CNC[C@@H]21]

Acetylchloride (4.59 g, 58.5 mmol) was cooled to below 0° C., dropwise added to ethanol, and stirred for 1 h at room temperature. Tert-butyl (1R,5S,6s)-6-(cyanomethyl)-3-azabicyclo[3.1.0]hexan-3-carboxylate (1.3 g, 5.85 mmol) was dissolved in ethanol (10 mL), and then added to the mixture obtained previously. The resulting mixture was heated to 70° C., and stirred for 24 hrs. The mixture was concentrated to obtain ethyl 2-((1R,5S,6s)-3-azabicyclo[3.1.0]hexan-6-yl)acetate hydrochloride (1.2 g, yield: 99.8%). The resulting crude product was directly used in the subsequent reaction. LC-MS: m/z=170.0 [M+H]⁺.

### Intermediate A2: Preparation of 2-azaspiro[3.3]heptan-6-carboxylic acid

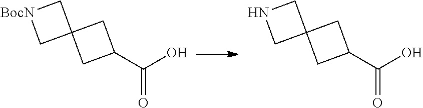 [SMILES: CC(C)(C)OC(=O)N1CC2(CC(C(=O)O)C2)C1 ; O=C(O)C1CC2(CNC2)C1]

2-(t-butyloxycarboryl)-2-azaspiro[3.3]heptan-6-carboxylic acid (500 mg, 2.07 mmol) was dissolved in trifluoroacetic acid (1 mL) and dichloromethane (3 mL), stirred for 1.5 hrs at room temperature, and concentrated to obtain 2-azaspiro[3.3]heptan-6-carboxylic acid (310 mg, yield: 99%). LC-MS: m/z=142.3 [M+H]⁺.

¹H NMR (400 MHz, DMSO-d₆) δ 8.64 (s, 2H), 3.97 (t, J=12.4 Hz, 2H), 3.89 (t, J=12.4 Hz, 2H), 2.89 (m, J=32.4 Hz, 1H), 2.42 (t, J=22.0 Hz, 2H), 2.31 (t, J=20.0 Hz, 2H).

Intermediates A3-A5 may be prepared by selecting corresponding starting materials by referring completely or in part to the synthesis method for Intermediates A1 or A2.

| Intermediate | | | MS m/z |
|---|---|---|---|
| No. | Structural Formula | Chemical Name | [M + 1]⁺ |
| A3 | 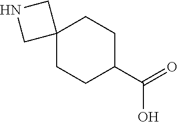 [SMILES: O=C(O)C1CCC2(CC1)CNC2] | 2-azaspiro[3.5] nonan-7- carboxylic acid | 170.1 |
| A4 | 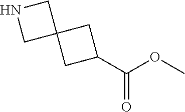 | methyl 2- azaspiro[3.3] heptan-6- carboxylate | 156.1 |
| A5 | 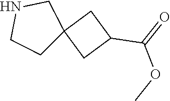 | methyl 6- azaspiro[3.4] octan-2- carboxylate | 170.1 |

### Intermediate A6: Preparation of ethyl (R)-2-((1R,5S,6S)-3-azabicyclo[3.1.0]hexan-6-yl)propionate

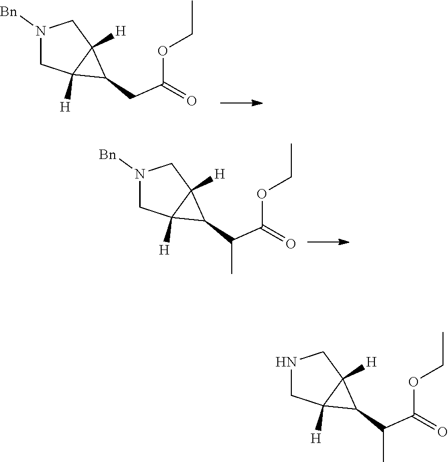 [SMILES: CCOC(=O)C[C@@H]1[C@H]2CN(Cc3ccccc3)C[C@@H]12 ; CCOC(=O)C(C)[C@H]1[C@@H]2CN(Cc3ccccc3)C[C@@H]21 ; CCOC(=O)C(C)[C@H]1[C@@H]2CNC[C@@H]21]

### Step 1: Synthesis of ethyl (R)-2-((1R,5S,6s)-3-benzyl-3-azabicyclo[3.1.0]hexan-6-yl)propionate

Ethyl 2-((1R,5S,6s)-3-benzyl-3-azabicyclo[3.1.0]hexan-6-yl)acetate (1.5 g, 5.79 mmol) was dissolved in tetrahydrofuran (28 mL); sodium bis(trimethylsilyl)amide (4.3 mL, 5.69 mmol, 1.5 equiv.) was added at minus 78° C., at which the reaction mixture was stirred for 45 min; and then, iodomethane (898 mg, 6.37 mmol, 1.1 equiv.) was added; and the resulting mixture was then stirred for 3 hrs at room temperature. LCMS showed that the reaction was completed. 20 mL of water was added to the reaction mixture for quenching; the mixture was then extracted with ethyl acetate (15 mL) three times, respectively; the organic phases were combined, then dried over anhydrous sodium sulfate, filtered, and spin-dried to obtain a crude product. The crude product was separated with forward silica gel columns (polarity:petroleum ether:ethyl acetate=3:1, Rf=0.6) to obtain ethyl (R)-2-((1R,5S,6s)-3-benzyl-3-azabicyclo[3.1.0]hexan-6-yl)propionate (830 mg, 3.04 mmol, yield: 52%). LCMS (ESI): [M+1]⁺=274.2.

### Step 2: Synthesis of ethyl (R)-2-((1R,5S,6s)-3-azabicyclo[3.1.0]hexan-6-yl)propionate

Ethyl (R)-2-((1R,5S,6s)-3-benzyl-3-azabicyclo[3.1.0]hexan-6-yl)propionate (830 mg, 3.04 mmol, 1.0 equiv.) was dissolved in ethanol (15 mL); palladium on carbon (10%) (644 mg, 6.08 mmol, 2.0 equiv.) was added; and the resulting mixture was stirred for 4 hrs at room temperature. LCMS showed that the reaction was completed. The reaction mixture was filtered, and the filtrate was spin-dried to obtain ethyl (R)-2-((1R,5S,6s)-3-azabicyclo[3.1.0]hexan-6-yl)propionate (520 mg, 2.84 mmol, yield: 93%). LCMS (ESI): [M+1]⁺=184.3.

### Intermediate B1: Preparation of 4-chloro-7,7-difluoro-2-(methylsulfonyl)-6,7-dihydro-5H-cyclopentadiene[d]pyrimidine

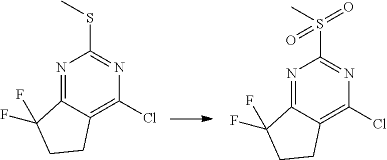 [SMILES: CSc1nc(Cl)c2c(n1)C(F)(F)CC2 ; CS(=O)(=O)c1nc(Cl)c2c(n1)C(F)(F)CC2]

4-chloro-7,7-difluoro-2-(methylthio)-6,7-dihydro-5H-cyclopenta[d]pyrimidine (1.0 g, 4.2 mmol) was dissolved in 8 ml of dichloromethane; a reagent m-CPBA (2.2 g, 85%, 10.5 mmol, 2.50 equiv.) was added; the resulting mixture was stirred for 3 hrs at room temperature, and LCMS showed that the reaction was performly completely; 5 ml of water was added to quench the reaction; 10 ml of sodium bicarbonate solution was added; extraction with dichloromethane was conducted twice (30 mL*2); and the organic phases were combined, washed with a salt solution, dried over sodium sulfate, filtered, and concentrated. The crude product was separated by silica gel column chromatography to obtain 4-chloro-7,7-difluoro-2-(methylsulfonyl)-6,7-dihydro-5H-cyclopentadiene[d]pyrimidine (1.1 g, yield: 96%). LCMS (ESI): [M+1]⁺=269.

### II. PREPARATION OF SPECIFIC EXAMPLES

### Example 1: Preparation of (S)-2-(5-cyano-6-(2-methylazetidine-1-yl)-4-(trifluoromethyl)pyridin-2-yl)-2-azaspiro[3.3]heptan-6-carboxylic acid

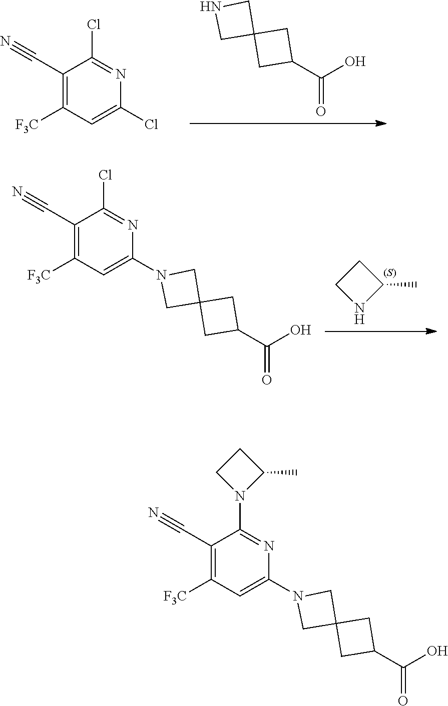

### Step 1: Synthesis of 2-(6-chloro-5-cyano-4-(trifluoromethyl)pyridin-2-yl)-2-azaspiro[3.3]heptan-6-carboxylic acid

2,6-dichloro-4-(trifluoromethyl)nicotinonitrile (650 mg, 2.71 mmol) and 2-azaspiro[3.3]heptan-6-carboxylic acid (300 mg, 2.12 mmol) were dissolved in DMF (5 mL); sodium bicarbonate (380 mg, 4.52 mmol) was added; and the resulting mixture was stirred for 1.5 hrs at room temperature. The mixture was filtered. The crude product was separated by preparative HPLC to obtain 2-(6-chloro-5-cyano-4-(trifluoromethyl)pyridin-2-yl)-2-azaspiro[3.3]heptan-6-carboxylic acid (200 mg, 0.58 mmol). LC-MS: m/z=346.0 [M+H]⁺.

### Step 2: Synthesis of (S)-2-(5-cyano-6-(2-methylazetidine-1-yl)-4-(trifluoromethyl)pyridin-2-yl)-2-azaspiro[3.3]heptan-6-carboxylic acid

2-(6-chloro-5-cyano-4-(trifluoromethyl)pyridin-2-yl)-2-azaspiro[3.3]heptan-6-carboxylic acid (100 mg, 0.29 mmol) and (S)-2-methylazetidine (44 mg, 0.41 mmol) were dissolved in DMF (2 mL); potassium carbonate (98 mg, 0.71 mmol) was added; and the resulting mixture was heated in an oil bath to 100° C. to react for 2 hrs. The mixture was cooled, filtered, and adjusted in pH to around 6 by using dilute hydrochloric acid, and extracted three times with EA (10 mL*3); the organic phases were combined, dried, and concentrated; and the resulting crude product was separated by preparative HPLC to obtain (S)-2-(5-cyano-6-(2-methylazetidine-1-yl)-4-(trifluoromethyl)pyridin-2-yl)-2-azaspiro[3.3]heptan-6-carboxylic acid (27.7 mg, 0.07 mmol, yield: 25%). LC-MS: m/z=381 [M+H]⁺.

¹H NMR (400 MHz, DMSO-d₆) δ 6.02 (s, 1H), 4.55 (m, J=20.8 Hz, 1H), 4.31 (m, J=22.4 Hz, 1H), 4.05 (m, J=31.6 Hz, 5H), 2.85 (t, J=15.6 Hz, 1H), 2.35 (m, J=53.2 Hz, 5H), 1.93 (m, J=20.0 Hz, 1H), 1.42 (d, J=6.0 Hz, 3H).

Examples 2-4 may be prepared by selecting corresponding starting materials by referring completely or in part to the synthesis method for Example 1.

| Example | | | MS m/z |
|---|---|---|---|
| No. | Structural Formula | Chemical Name | [M + 1]⁺ |
| 2 | 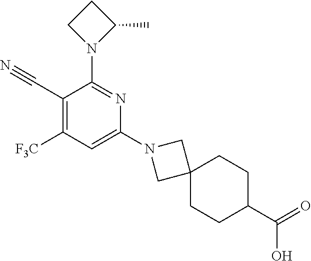 [SMILES: C[C@H]1CCN1c1nc(N2CC3(CCC(C(=O)O)CC3)C2)cc(C(F)(F)F)c1C#N] | (S)-2-(5-cyano-6-(2- methylazetidine-1-yl)-4- (trifluoromethyl)pyridin-2-yl)-2- azaspiro[3.5]nonan-7-carboxylic acid | 409 |
| 3 | 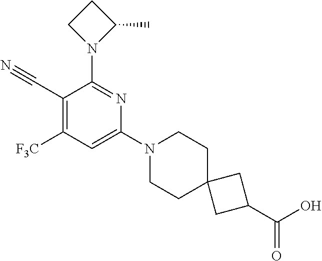 [SMILES: C[C@H]1CCN1c1nc(N2CCC3(CC2)CC(C(=O)O)C3)cc(C(F)(F)F)c1C#N] | (S)-7-(5-cyano-6-(2- methylazetidine-1-yl)-4- (trifluoromethyl)pyridin-2-yl)-7- azaspiro[3.5]nonan-2-carboxylic acid | 409 |
| 4 | 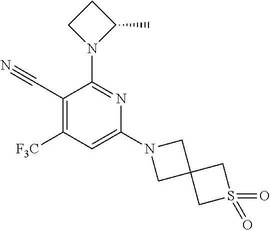 [SMILES: C[C@H]1CCN1c1nc(N2CC3(C2)CS(=O)(=O)C3)cc(C(F)(F)F)c1C#N] | (S)-6-(2,2-dioxa-2-thioxa-6- azaspiro[3.3]heptan-6-yl)-2-(2- methylazetidine-1-yl)-4- (trifluoromethyl)nicotinonitrile | 387 |

### Example 5: Preparation of methyl 2-(5-cyano-6-((2S,3R)-3-hydroxy-2-methylazetidine-1-yl)-4-(trifluoromethyl)pyridin-2-yl)-2-azaspiro[3.3]heptan-6-carboxylate

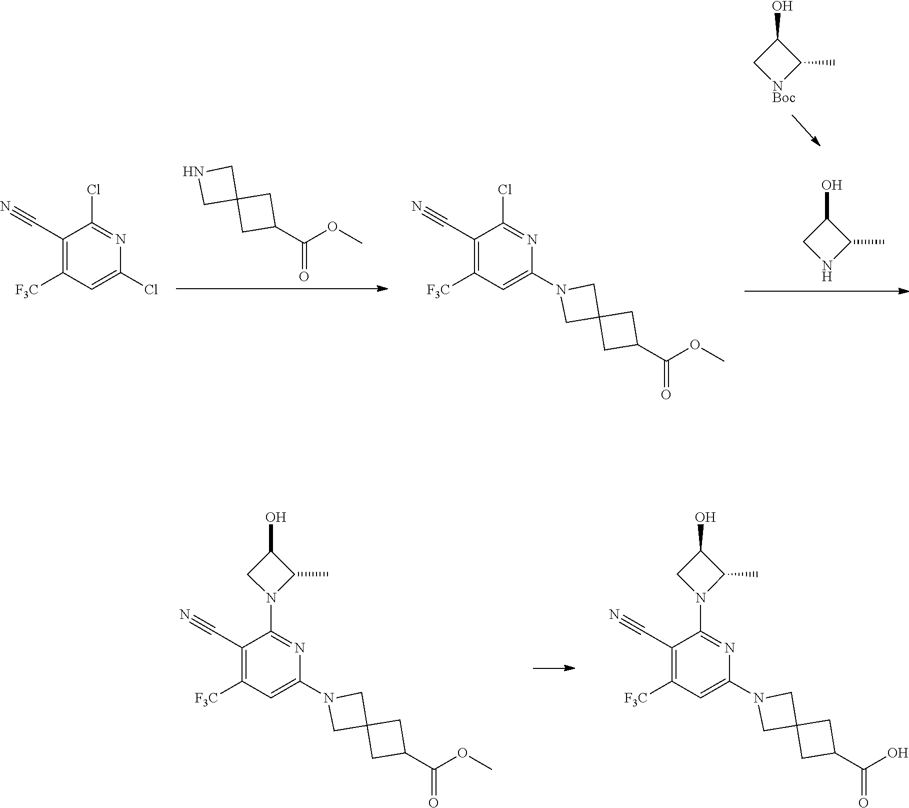

### Step 1: Synthesis of (2S,3R)-2-methylazetidine-3-ol

Tert-butyl (2S,3R)-3-hydroxy-2-methylazetidine-1-carboxylate (112.0 mg, 0.60 mmol) was dissolved in dichloromethane (3 mL); trifluoroacetic acid (1 mL) was dropwise added; the resulting mixture was stirred for 1 hr at room temperature, and evaporated to remove the solvent under reduced pressure to obtain (2S,3R)-2-methylazetidine-3-ol (106.0 mg, yield: 95.6%); and the resulting crude product was directly used in the next step of reaction.

### Step 2: Synthesis of methyl 2-(6-chloro-5-cyano-4-(trifluoromethyl)pyridin-2-yl)-2-azaspiro[3.3]heptan-6-carboxylate

2,6-dichloro-4-(trifluoromethyl)nicotinonitrile (230 mg, 0.95 mmol) and methyl 2-azaspiro[3.3]heptan-6-carboxylate (240.0 mg, 0.95 mmol) were dissolved in acetonitrile (5 mL); potassium carbonate (395.1 mg, 2.86 mmol) was added; and the reaction mixture was heated to 35° C., and stirred for 2 hrs. LCMS showed that starting materials disappeared and the reaction occurred completely; the mixture was filtered and concentrated; and the resulting crude product was separated by silica gel column chromatography to obtain methyl 2-(6-chloro-5-cyano-4-(trifluoromethyl)pyridin-2-yl)-2-azaspiro[3.3]heptan-6-carboxylate (240 mg, 0.67 mmol, yield: 70%). LCMS(ESI): [M+1]⁺=359.9.

¹H NMR (400 MHz, CDCl₃) δ 6.33 (s, 1H), 4.21 (d, J=8.3 Hz, 4H), 3.71 (d, J=5.5 Hz, 3H), 3.08 (p, J=7.9 Hz, 1H), 2.55 (d, J=7.9 Hz, 4H).

### Step 3: Synthesis of methyl 2-(5-cyano-6-((2S,3R)-3-hydroxy-2-methylazetidine-1-yl)-4-(trifluoromethyl)pyridin-2-yl)-2-azaspiro[3.3]heptan-6-carboxylate

Methyl 2-(6-chloro-5-cyano-4-(trifluoromethyl)pyridin-2-yl)-2-azaspiro[3.3]heptan-6-carboxylate (190.0 mg, 0.53 mmol) and (2S,3R)-2-methylazetidine-3-ol (106.0 mg, 0.57 mmol) were dissolved in acetonitrile (5 ml); potassium carbonate (219.1 mg, 1.59 mmol) was added; the reaction mixture was heated to 75° C., and stirred for 2 hrs; LCMS showed that starting materials disappeared and the reaction occurred completely; the reaction mixture was filtered and concentrated; the resulting crude product was separated by silica gel column chromatography to obtain methyl 2-(5-cyano-6-((2S,3R)-3-hydroxy-2-methylazetidine-1-yl)-4-(trifluoromethyl)pyridin-2-yl)-2-azaspiro[3.3]heptan-6-carboxylate (210 mg, 0.51 mmol, yield: 97%). LCMS(ESI): [M+1]⁺=411.0.

¹H NMR (400 MHz, CDCl₃) δ 5.84 (s, 1H), 4.76 (ddd, J=9.4, 6.6, 1.0 Hz, 1H), 4.41-4.29 (m, 1H), 4.27-4.18 (m, 1H), 4.15-3.96 (m, 4H), 3.91 (dd, J=9.5, 4.8 Hz, 1H), 3.70 (s, 3H), 3.13-2.97 (m, 1H), 2.57-2.45 (m, 4H), 1.48 (d, J=6.4 Hz, 3H).

### Step 4: Synthesis of 2-(5-cyano-6-((2S,3R)-3-hydroxy-2-methylazetidine-1-yl)-4-(trifluoromethyl)pyridin-2-yl)-2-azaspiro[3.3]heptan-6-carboxylic acid

Methyl 2-(5-cyano-6-((2S,3R)-3-hydroxy-2-methylazetidine-1-yl)-4-(trifluoromethyl)pyridin-2-yl)-2-azaspiro[3.3]heptan-6-carboxylate (95.0 mg, 0.23 mmol) was dissolved in tetrahydrofuran (2 ml); and lithium hydroxide aqueous solution (2M, 2 mL) was added; and the reaction mixture was reacted for 2 hrs at room temperature, with LCMS showing that starting materials disappeared and the reaction occurred completely. 1N hycrochloric acid was dropwise added to neutrilize the reaction system; the reaction mixture was filtered and concentrated; and the resulting crude produce was separated by preparative HPLC to obtain 2-(5-cyano-6-((2S,3R)-3-hydroxy-2-methylazetidine-1-yl)-4-(trifluoromethyl)pyridin-2-yl)-2-azaspiro[3.3]heptan-6-carboxylic acid (50 mg, 0.13 mmol, yield: 55%). LCMS(ESI): [M+1]+=397.2.

¹H NMR (400 MHz, DMSO-d₆) δ 12.06 (s, 1H), 6.07 (s, 1H), 5.65 (s, 1H), 4.60-4.47 (m, 1H), 4.24-4.15 (m, 1H), 4.17-3.93 (m, 5H), 3.73 (dd, J=8.9, 4.9 Hz, 1H), 2.95 (dd, J=16.4, 8.2 Hz, 1H), 2.48-2.30 (m, 4H), 1.40 (d, J=6.4 Hz, 3H).

¹⁹F NMR (377 MHz, DMSO-d₆) δ −63.69 (s, 3F).

Examples 6-14 may be prepared by selecting corresponding starting materials by referring completely or in part to the synthesis method for Example 5.

| Example | | | MS m/z |
|---|---|---|---|
| No. | Structural Formula | Chemical Name | [M + 1]⁺ |
| 6 | 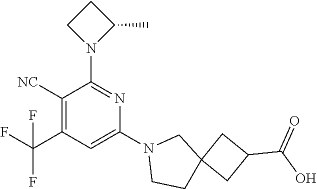 [SMILES: C[C@H]1CCN1c1nc(N2CCC3(CC(C(=O)O)C3)C2)cc(C(F)(F)F)c1C#N] | (S)-6-(5-cyano-6-(2-methylazetidine- 1-yl)-4-(trifluoromethyl)pyridin- 2-yl)-6-azaspiro[3.4]octan-2- carboxylic acid | 395 |
| 7 | 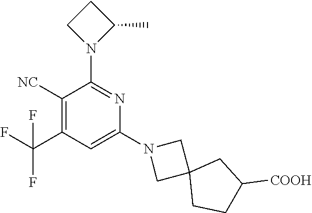 [SMILES: C[C@H]1CCN1c1nc(N2CC3(CCC(C(=O)O)C3)C2)cc(C(F)(F)F)c1C#N] | 2-(5-cyano-6-((S)-2-methylazetidine- 1-yl)-4-(trifluoromethyl)pyridin-2-yl)- 2-azaspiro[3.4]octan-6-carboxylic acid | 395 |
| 8 | 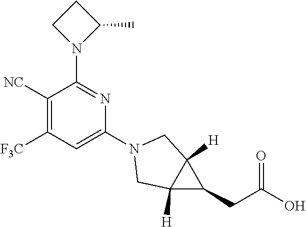 [SMILES: C[C@H]1CCN1c1nc(N2C[C@@H]3[C@@H](CC(=O)O)[C@@H]3C2)cc(C(F)(F)F)c1C#N] | 2-((1R,5S,6R)-3-(5-cyano-6-((S)- 2-methylazetidine-1-yl)-4- (trifluoromethyl)pyridin-2-yl)-3- azabicyclo[3.1.0]hexan-6-yl)acetic acid | 381 |
| 9 | 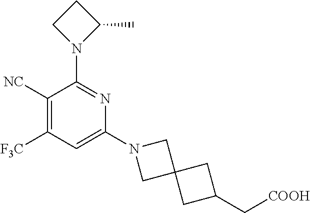 [SMILES: C[C@H]1CCN1c1nc(N2CC3(CC(CC(=O)O)C3)C2)cc(C(F)(F)F)c1C#N] | (S)-2-(2-(5-cyano-6-(2-methylazetidine- 1-yl)-4-(trifluoromethyl)pyridin-2- yl)-2-azaspiro[3.3]heptan-6-yl) acetic acid | 395 |
| 10 | 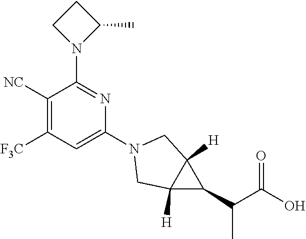 [SMILES: CC(C(=O)O)[C@H]1[C@@H]2CN(c3cc(C(F)(F)F)c(C#N)c(N4CC[C@@H]4C)n3)C[C@@H]21] | (R)-2-((1R,5S,6R)-3-(5-cyano-6- ((S)-2-methylazetidine-1-yl)-4- (trifluoromethyl)pyridin-2-yl)-3- azabicyclo[3.1.0]hexan-6-yl) propionic acid | 395 |
| 11 | 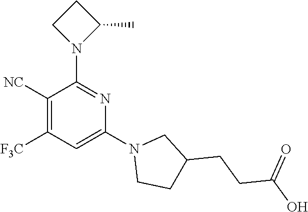 [SMILES: C[C@H]1CCN1c1nc(N2CCC(CCC(=O)O)C2)cc(C(F)(F)F)c1C#N] | 3-(1-(5-cyano-6-((S)-2-methylazetidine- 1-yl)-4-(trifluoromethyl) pyridin-2-yl)pyrrolidin-3-yl) propionic acid | 383 |
| 12 | 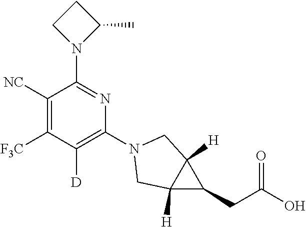 [SMILES: [2H]c1c(N2C[C@@H]3[C@@H](CC(=O)O)[C@@H]3C2)nc(N2CC[C@@H]2C)c(C#N)c1C(F)(F)F] | 2-((1R,5S,6R)-3-(5-cyano-6-((S)- 2-methylazetidine-1-yl)-4- (trifluoromethyl)pyridin-2-yl-3-d)- 3-azabicyclo[3.1.0]hexan-6-yl) acetic acid | 382 |
| 13 | 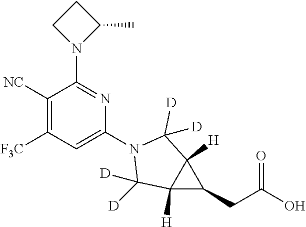 [SMILES: [2H]C1([2H])[C@@H]2[C@@H](CC(=O)O)[C@@H]2C([2H])([2H])N1c1cc(C(F)(F)F)c(C#N)c(N2CC[C@@H]2C)n1] | 2-((1R,5S,6R)-3-(5-cyano-6-((S)- 2-methylazetidine-1-yl)-4- (trifluoromethyl)pyridin-2-yl)-3- azabicyclo[3.1.0]hexan-6-yl-2,2, 4,4-d₄)acetic acid | 385 |
| 14 | 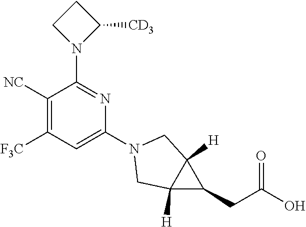 [SMILES: [2H]C([2H])([2H])[C@H]1CCN1c1nc(N2C[C@@H]3[C@@H](CC(=O)O)[C@@H]3C2)cc(C(F)(F)F)c1C#N] | 2-((1R,5S,6R)-3-(5-cyano-6-((S)- 2-(methyl-d₃)azetidine-1-yl)-4- (trifluoromethyl)pyridin-2-yl)-3- azabicyclo[3.1.0]hexan-6-yl) acetic acid | 384 |

### Example 15: Preparation of (S)-2-(6-(5-cyano-6-(2-methylazetidine-1-yl)-4-(trifluoromethyl)pyridin-2-yl)-2,6-diazaspiro[3.3]heptan-2-yl)acetic acid

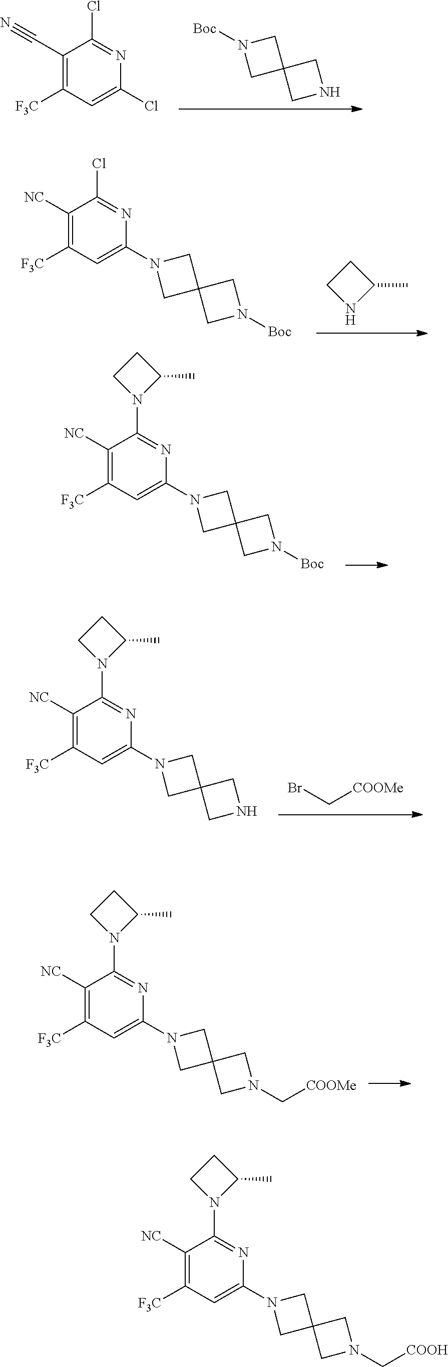

### Step 1: Synthesis of tert-butyl 6-(6-chloro-5-cyano-4-(trifluoromethyl)pyridin-2-yl)-2,6-diazaspiro[3.3]heptan-2-carboxylate

2,6-dichloro-4-(trifluoromethyl)nicotinonitrile (200.0 mg, 0.83 mmol) and tert-butyl 2,6-diazaspiro[3.3]heptan-2-carboxylate (164.0 mg, 0.83 mmol, 1.00 equiv) were dissolved in acetonitrile (4 mL); potassium carbonate (343.0 mg, 2.49 mmol, 3.00 equiv) was added; the resulting mixture was stirred for 18 hrs at room temperature. The reaction mixture was concentrated under reduced pressure to obtain an oily product, which was separated by flash column chromatography to obtain tert-butyl 6-(6-chloro-5-cyano-4-(trifluoromethyl)pyridin-2-yl)-2,6-diazaspiro[3.3]heptan-2-carboxylate (270.0 mg, yield: 80.8%).

### Step 2: Synthesis of tert-butyl (S)-6-(5-cyano-6-(2-methylazetidine-1-yl)-4-(trifluoromethyl)pyridin-2-yl)-2,6-diazaspiro[3.3]heptan-2-carboxylate

(S)-2-(6-(5-cyano-6-(2-methylazetidine-1-yl)-4-(trifluoromethyl)pyridin-2-yl)-2,6-diazaspiro[3.3]heptan-2-yl)acetic acid (270.0 mg, 0.67 mmol) and (S)-2-methylazetidine (48.0 mg, 0.67 mmol, 1.00 equiv) were dissolved in acetonitrile (5 mL); and potassium carbonate (277.0 mg, 2.01 mol, 3.00 equiv) was added. The reaction mixture was stirred for 18 hrs at room temperature. The reaction mixture was concentrated under reduced pressure to obtain an oily product, which was separated by flash column chromatography to obtain tert-butyl (S)-6-(5-cyano-6-(2-methylazetidine-1-yl)-4-(trifluoromethyl)pyridin-2-yl)-2,6-diazaspiro[3.3]heptan-2-carboxylate (120 mg, yield: 41.3%).

### Step 3: Synthesis of (S)-2-(2-methylazetidine-1-yl)-6-(2,6-diazaspiro[3.3]heptan-2-yl)-4-(trifluoromethyl)nicotinonitrile

Tert-butyl (S)-6-(5-cyano-6-(2-methylazetidine-1-yl)-4-(trifluoromethyl)pyridin-2-yl)-2,6-diazaspiro[3.3]heptan-2-carboxylate (230.0 mg, 0.53 mmol) was dissolved in the mixed solution of dichloromethane (3 mL) and trifluoroacetic acid (1 mL). The mixture was stirred for 1 hour at room temperature. The reaction mixture was concentrated under reduced pressure to obtain an oily product, (S)-2-(2-methylazetidine-1-yl)-6-(2,6-diazaspiro[3.3]heptan-2-yl)-4-(trifluoromethyl)nicotinonitrile (200.0 mg, yield: 87.6%). The oily product was directly used in the next step of reaction.

### Step 4: Synthesis of methyl (S)-2-(6-(5-cyano-6-(2-methylazetidine-1-yl)-4-(trifluoromethyl)pyridin-2-yl)-2,6-diazaspiro[3.3]heptan-2-yl)acetate

(S)-2-(2-methylazetidine-1-yl)-6-(2,6-diazaspiro[3.3]heptan-2-yl)-4-(trifluoromethyl)nicotinonitrile (100.0 mg, 0.30 mmol) and methyl 2-bromoacetate (60.0 mg, 0.36 mmol, 1.20 equiv) were dissolved in acetonitrile (5 mL); and potassium carbonate (123.0 mg, 0.89 mmol, 3.00 equiv) was added. The mixture was stirred for 1 hour at room temperature. The reaction mixture was concentrated under reduced pressure to obtain an oily product, which was separated by flash column chromatography to obtain methyl (S)-2-(6-(5-cyano-6-(2-methylazetidine-1-yl)-4-(trifluoromethyl)pyridin-2-yl)-2,6-diazaspiro[3.3]heptan-2-yl)acetate (100 mg, yield: 82.4%).

### Step 5: Synthesis of (S)-2-(6-(5-cyano-6-(2-methylazetidine-1-yl)-4-(trifluoromethyl)pyridin-2-yl)-2,6-diazaspiro[3.3]heptan-2-yl)acetic acid

Methyl (S)-2-(6-(5-cyano-6-(2-methylazetidine-1-yl)-4-(trifluoromethyl)pyridin-2-yl)-2,6-diazaspiro[3.3]heptan-2-yl)acetate (60.0 mg, 0.15 mmol, 1.00 equiv) was dissolved in the mixed solution of tetrahydrofuran (2 mL) and methanol (2 mL); lithium hydroxide aqueous solution (2M, 2 mL) was added; and the resulting mixture was stirred for 1 hour at room temperature. The reaction mixture was concentrated under reduced pressure to obtain an oily product, which was separated by reverse-phase preparation to obtain (S)-2-(6-(5-cyano-6-(2-methylazetidine-1-yl)-4-(trifluoromethyl)pyridin-2-yl)-2,6-diazaspiro[3.3]heptan-2-yl)acetic acid (24.0 mg yield: 46.5%).

¹H NMR (400 MHz, DMSO-d₆) δ 6.06 (s, 1H), 4.55 (m, J=14.4, 6.5 Hz, 1H), 4.31 (m, J=8.9, 5.2 Hz, 1H), 4.16 (s, 3H), 4.08-3.94 (m, 1H), 3.69 (s, 5H), 3.16 (d, J=6.0 Hz, 2H), 2.46-2.32 (m, 1H), 1.93 (m, J=14.0, 5.6 Hz, 1H), 1.41 (t, J=9.1 Hz, 3H).

¹⁹F NMR (377 MHz, DMSO-d₆) δ −63.78 (d, J=94.9 Hz).

### Example 16: Preparation of (S)-6-(5-aminoformyl-6-(2-methylazetidine-1-yl)-4-(trifluoromethyl)pyridin-2-yl)-6-azaspiro[3.4]octan-2-carboxylic acid

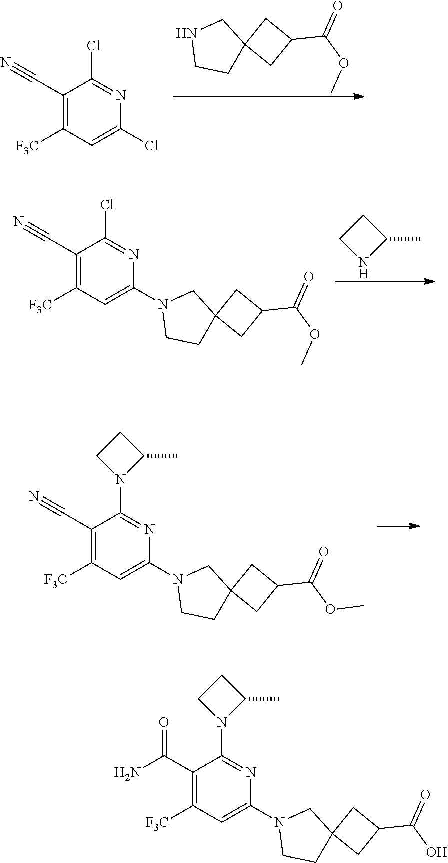

Referring to Steps 1-3 in Example 5, methyl (S)-6-(5-cyano-6-(2-methylazetidine-1-yl)-4-(trifluoromethyl)pyridin-2-yl)-6-azaspiro[3.4]octan-2-carboxylate was synthesized; methyl (S)-6-(5-cyano-6-(2-methylazetidine-1-yl)-4-(trifluoromethyl)pyridin-2-yl)-6-azaspiro[3.4]octan-2-carboxylate was dissolved in DMSO (2 mL); potassium hydroxide (49.0 mg, 0.88 mmol, 4.00 equiv) was added; H₂O₂ (2 mL) is dropwise added; and the resulting mixture was heated to 50° C. and stirred for 48 hrs. LCMS showed that the reaction occurred completely. Saturated sodium thiosulfate solution was added to quench the reaction, the mixture was concentrated under reduced pressure; and a resulting crude product was preparatively separated by HPLC to obtain (S)-6-(5-aminoformyl-6-(2-methylazetidine-1-yl)-4-(trifluoromethyl)pyridin-2-yl)-6-azaspiro[3.4]octan-2-carboxylic acid (2.2 mg, 5.00 μmol, yield: 2.44%). LCMS(ESI): [M+1]⁺=413.4. tR=1.239 min.

¹H NMR (400 MHz, DMSO-d₆) δ 7.57 (s, 1H), 7.39 (s, 1H), 5.84 (d, J=10.7 Hz, 1H), 4.45 (s, 1H), 4.02 (s, 1H), 3.84 (d, J=8.1 Hz, 1H), 3.42 (m, 4H), 3.10-3.00 (m, 1H), 2.27 (s, 1H), 2.15 (t, J=9.4 Hz, 4H), 2.00 (d, J=6.9 Hz, 1H), 1.94-1.82 (m, 2H), 1.42 (d, J=6.0 Hz, 3H).

### Example 17: Preparation of (S)-6-(2,2-dioxa-2-thioxa-6-azaspiro[3.3]heptan-6-yl)-2-(2-methylazetidine-1-yl)-4-(trifluoromethyl)nicotinamide

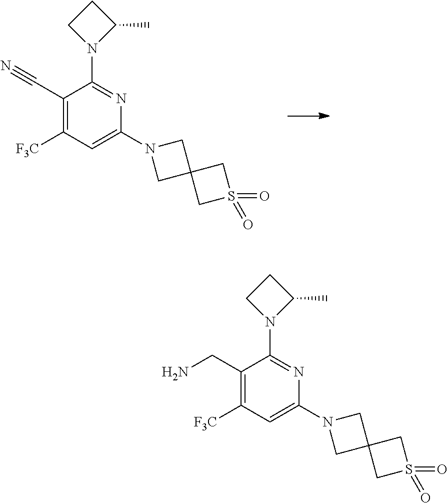 [SMILES: C[C@H]1CCN1c1nc(N2CC3(C2)CS(=O)(=O)C3)cc(C(F)(F)F)c1C#N ; C[C@H]1CCN1c1nc(N2CC3(C2)CS(=O)(=O)C3)cc(C(F)(F)F)c1CN]

(S)-6-(2,2-dioxa-2-thioxa-6-azaspiro[3.3]heptan-6-yl)-2-(2-methylazetidine-1-yl)-4-(trifluoromethyl)nicotinonitrile (25.0 mg, 64.75 μmol) was dissolved in DMSO (1 mL); potassium hydroxide (5.4 mg, 97.12 μmol, 1.50 equiv) was added; H₂O₂ (30% wt, 0.5 mL) was dropwise added; and the resulting mixture was heated to 50° C., and stirred for 6 hrs, with the LCMS showing that the conversion rate was 20% after reaction. H₂O₂ (30% wt, 0.5 mL) was dropwise added; the mixture was heated to 50° C., and stirred for 6 hrs, with LCMS showing that the conversion rate was 50% after reaction. H₂O₂ (30% wt, 0.5 mL) was dropwise added; the mixture was heated to 50° C., and stirred for 18 hrs, with LCMS showing that the conversion rate was 76% after reaction. The reaction mixture was diluted with water (2 ml), extracted with ethyl acetate (5 mL*2); the organic phases were washed with saturated NaCl solutions, dried, filtered, and concentrated under reduced pressure; and the resulting crude product was separated by preparative HPLC to obtain (S)-6-(2,2-dioxa-2-thioxa-6-azaspiro[3.3]heptan-6-yl)-2-(2-methylazetidine-1-yl)-4-(trifluoromethyl)nicotinamide (7.1 mg, yield: 27.1%). LCMS(ESI): [M+1]=405.0. tR=1.197 min.

¹H NMR (400 MHz, DMSO-d₆) δ 7.68 (s, 1H), 7.49 (s, 1H), 5.87 (d, J=1.8 Hz, 1H), 4.55-4.38 (m, 5H), 4.17 (q, J=8.6 Hz, 4H), 4.04 (s, 1H), 3.89 (q, J=8.2 Hz, 1H), 2.31 (s, 1H), 1.89 (t, J=8.9 Hz, 1H), 1.41 (dd, J=6.2, 1.7 Hz, 3H).

### Examples 18 and 19: Preparation of (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid and (S)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid

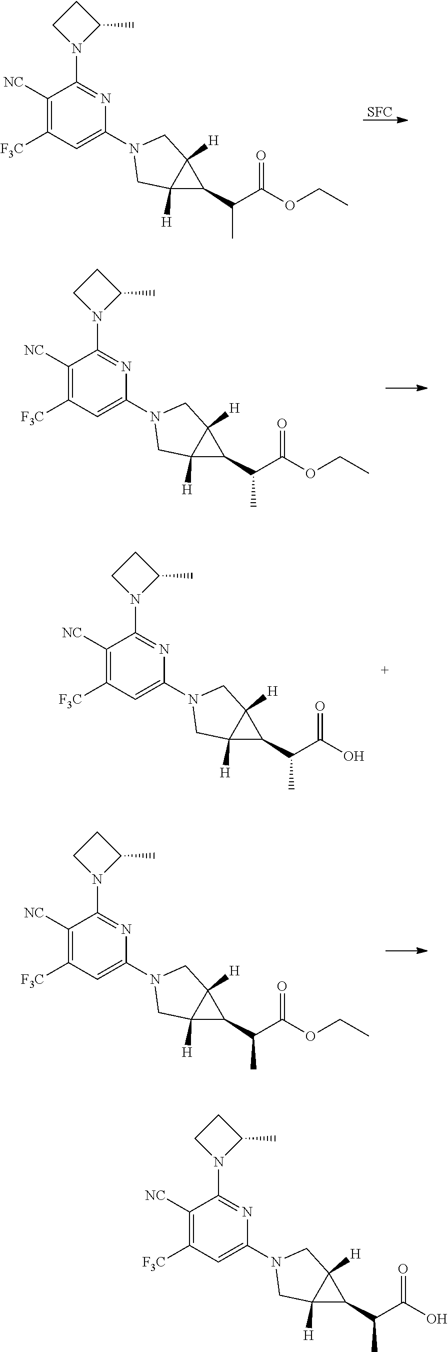 [SMILES: CCOC(=O)C(C)[C@H]1[C@@H]2CN(c3cc(C(F)(F)F)c(C#N)c(N4CC[C@@H]4C)n3)C[C@@H]21 ; "SFC" ; CCOC(=O)[C@H](C)[C@H]1[C@@H]2CN(c3cc(C(F)(F)F)c(C#N)c(N4CC[C@@H]4C)n3)C[C@@H]21 ; C[C@@H](C(=O)O)[C@H]1[C@@H]2CN(c3cc(C(F)(F)F)c(C#N)c(N4CC[C@@H]4C)n3)C[C@@H]21 ; CCOC(=O)[C@@H](C)[C@H]1[C@@H]2CN(c3cc(C(F)(F)F)c(C#N)c(N4CC[C@@H]4C)n3)C[C@@H]21 ; C[C@H](C(=O)O)[C@H]1[C@@H]2CN(c3cc(C(F)(F)F)c(C#N)c(N4CC[C@@H]4C)n3)C[C@@H]21]

### Step 1: Chiral resolution of ethyl (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate

Referring to Steps 1-3 in Example 5, ethyl (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate was synthesized, and ethyl (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate (400 mg) was chirally resolved to obtain ethyl (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate (150 mg, retention time: 1.835 min) and ethyl (S)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate (150 mg, retention time: 2.467 min).
Instrument: Waters Thar80 preparative SFC
Column: ChiralPak C-IG, 250*21.2 mm I.D., 5 μm
Mobile phase: A for CO₂ and B for MEOH+0.1% NH₃H₂O
Gradient: B 20%, Flow rate: 40 mL/min, Back pressure: 100 bar,
Column temperature: 35° C., Wavelength: 254 nm, Cycle-time: 20 min
Eluted time: 1.5 H.

### Step 2: Synthesis of (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid

Ethyl (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate (150 mg, 0.36 mmol) was dissolved in a mixed solvent of ethanol (2.5 mL) and water (0.5 mL); sodium hydroxide (115 mg, 2.88 mmol, 8.0 equiv.) was added; and then the resulting mixture was stirred for 5 hrs at room temperature stirred. LCMS showed that the reaction was completed. The reaction mixture was concentrated under reduced pressure, and the resulting crude product was separated by high-performance liquid chromatography prepared with a formic acid system and freeze-dried to obtain (R)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid (32.2 mg, 0.082 mmol, yield: 23%). LCMS (ESI): [M+1]⁺=395.3.

¹H NMR (400 MHz, CD₃OD) δ 6.08 (s, 1H), 4.70-4.56 (m, 1H), 4.42 (td, J=8.9, 5.0 Hz, 1H), 4.08 (dd, J=16.0, 8.9 Hz, 1H), 4.01-3.39 (m, 4H), 2.51-2.37 (m, 1H), 2.06-1.92 (m, 1H), 1.87-1.76 (m, 1H), 1.70 (brs, 1H), 1.58 (brs, 1H), 1.49 (d, J=6.2 Hz, 3H), 1.22 (d, J=7.0 Hz, 3H), 0.74 (dt, J=9.5, 3.4 Hz, 1H).

### Step 3: Synthesis of (S)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid

Ethyl (S)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionate (150 mg, 0.36 mmol) was dissolved in a mixed solvent of ethanol (2.5 mL) and water (0.5 mL); sodium hydroxide (115 mg, 2.88 mmol, 8.0 equiv.) was added; and the resulting mixture was stirred for 5 hrs at room temperature stirred. LCMS showed that the reaction was completed. The reaction mixture was concentrated under reduced pressure, and the resulting crude product was separated by high-performance liquid chromatography prepared with a formic acid system, and freeze-dried to obtain (S)-2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)propionic acid (30 mg, 0.076 mmol, yield: 21.2%). LCMS (ESI): [M+1]⁺=395.3.

¹H NMR (400 MHz, CD₃OD) δ 6.08 (s, 1H), 4.72-4.56 (m, 1H), 4.47-4.35 (m, 1H), 4.07 (dt, J=15.9, 8.0 Hz, 1H), 4.01-3.76 (m, 1H), 3.66-3.40 (m, 3H), 2.52-2.36 (m, 1H), 2.05-1.91 (m, 1H), 1.80-1.60 (m, 2H), 1.53 (brs, 1H), 1.49 (d, J=6.2 Hz, 3H), 1.22-1.15 (m, 3H), 0.75 (dt, J=9.4, 3.5 Hz, 1H).

### Examples 20 and 21: Preparation of 3-((S)-1-(5-cyano-6-((S)-2-methylazetidine-1-yl)-4-(trifluoromethyl)pyridin-2-yl)pyrrolidin-3-yl)propionic acid and 3-((R)-1-(5-cyano-6-((S)-2-methylazetidine-1-yl)-4-(trifluoromethyl)pyridin-2-yl)pyrrolidin-3-yl)propionic acid

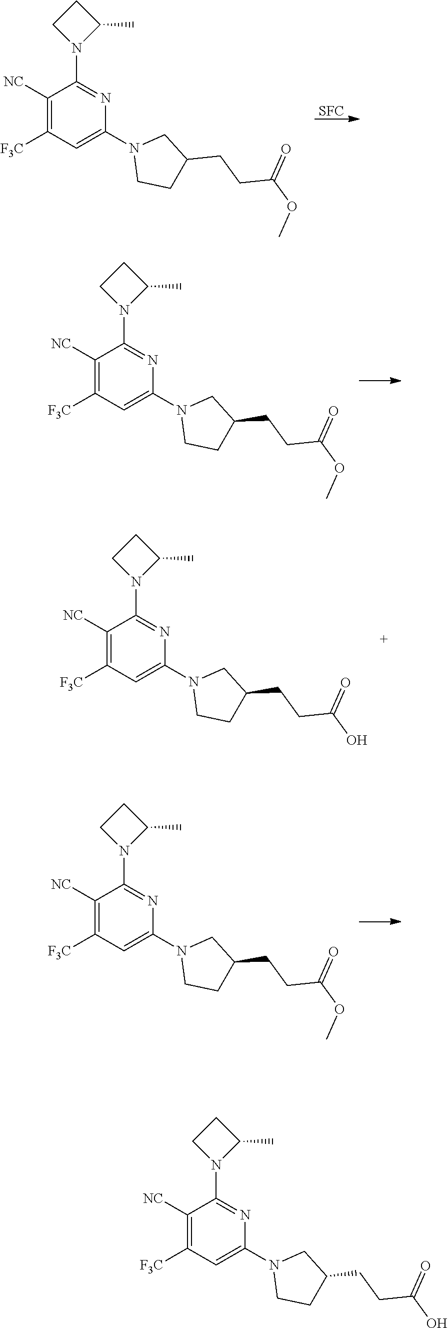 [SMILES: COC(=O)CCC1CCN(c2cc(C(F)(F)F)c(C#N)c(N3CC[C@@H]3C)n2)C1 ; "SFC" ; COC(=O)CC[C@H]1CCN(c2cc(C(F)(F)F)c(C#N)c(N3CC[C@@H]3C)n2)C1 ; C[C@H]1CCN1c1nc(N2CC[C@H](CCC(=O)O)C2)cc(C(F)(F)F)c1C#N ; COC(=O)CC[C@H]1CCN(c2cc(C(F)(F)F)c(C#N)c(N3CC[C@@H]3C)n2)C1 ; C[C@H]1CCN1c1nc(N2CC[C@@H](CCC(=O)O)C2)cc(C(F)(F)F)c1C#N]

### Step 1: Chiral resolution of methyl 3-((S)-1-(5-cyano-6-((S)-2-methylazetidine-1-yl)-4-(trifluoromethyl)pyridin-2-yl)pyrrolidin-3-yl)propionate and methyl 3-((R)-1-(5-cyano-6-((S)-2-methylazetidine-1-yl)-4-(trifluoromethyl)pyridin-2-yl)pyrrolidin-3-yl)propionate

Referring to Steps 1-3 in Example 5, 3-(1-(5-cyano-6-((S)-2-methylazetidine-1-yl)-4-(trifluoromethyl)pyridin-2-yl)pyrrolidin-3-yl)methyl propionate was synthesized, and 3-(1-(5-cyano-6-((S)-2-methylazetidine-1-yl)-4-(trifluoromethyl)pyridin-2-yl)pyrrolidin-3-yl)methyl propionate (380 mg) was chiraly resolved to obtain methyl 3-((S)-1-(5-cyano-6-((S)-2-methylazetidine-1-yl)-4-(trifluoromethyl)pyridin-2-yl)pyrrolidin-3-yl)propionate (93 mg, retention time: 3.908 min) and methyl 3-((R)-1-(5-cyano-6-((S)-2-methylazetidine-1-yl)-4-(trifluoromethyl)pyridin-2-yl)pyrrolidin-3-yl)propionate (135 mg, retention time: 4.911 min).
Instrument: CAS-SH-ANA-HPLC-Z (Shimadzu LC-20AD with PDA detector)
Acq Method: D: method\PA_DEA\H_IPA_DEA_20_1ML_10MIN_10CM.lcm
Column: Chiralpak IH 100*4.6 mm 3 um
Mobile phase: A: Hexane (0.1% DEA) B: IPA
Isocratic: A/B=80/20; Flow Rate: 1 mL/min; Column Temp.: 35° C.

### Step 2: Separation of 3-((S)-1-(5-cyano-6-((S)-2-methylazetidine-1-yl)-4-(trifluoromethyl)pyridin-2-yl)pyrrolidin-3-yl)propionic acid

3-((S)-1-(5-cyano-6-((S)-2-methylazetidine-1-yl)-4-(trifluoromethyl)pyridin-2-yl)pyrrolidin-3-yl)propionate (93 mg, 0.23 mmol) was dissolved in a mixed solvent of methanol (2.5 mL) and water (0.5 mL); sodium hydroxide (74 mg, 1.84 mmol, 8.0 equiv.) was added and the resulting mixture was stirred for 5 hrs at room temperature stirred. LCMS showed that the reaction was completed. The reaction mixture was concentrated under reduced pressure, and the resulting crude product was separated by high-performance liquid chromatography prepared with a formic acid system, and freeze-dried to obtain 3-((S)-1-(5-cyano-6-((S)-2-methylazetidine-1-yl)-4-(trifluoromethyl)pyridin-2-yl)pyrrolidin-3-yl)propionic acid (23 mg, 0.060 mmol, yield: 26.2%). LCMS (ESI): [M+1]⁺=383.4.

¹H NMR (400 MHz, CD₃OD) δ 6.13 (s, 1H), 4.72-4.58 (m, 1H), 4.42 (td, J=8.9, 5.1 Hz, 1H), 4.08 (dd, J=15.9, 8.9 Hz, 1H), 3.86-3.34 (m, 3H), 3.24-2.92 (m, 1H), 2.54-2.39 (m, 1H), 2.37-2.08 (m, 4H), 2.04-1.91 (m, 1H), 1.83-1.56 (m, 3H), 1.50 (d, J=6.2 Hz, 3H).

### Step 3: Separation of 3-((R)-1-(5-cyano-6-((S)-2-methylazetidine-1-yl)-4-(trifluoromethyl)pyridin-2-yl)pyrrolidin-3-yl)propionic acid

Methyl 3-((R)-1-(5-cyano-6-((S)-2-methylazetidine-1-yl)-4-(trifluoromethyl)pyridin-2-yl)pyrrolidin-3-yl)propionate (135 mg, 0.34 mmol) was dissolved in a mixed solvent of methanol (2.5 mL) and water (0.5 mL); sodium hydroxide (109 mg, 2.73 mmol, 8.0 equiv.) was added; and the resulting mixture was stirred for 5 hrs at room temperature. LCMS showed that the reaction was completed. The reaction mixture was concentrated under reduced pressure, and the resulting crude product was separated by high-performance liquid chromatography prepared with a formic acid system, and freeze-dried to obtain 3-((R)-1-(5-cyano-6-((S)-2-methylazetidine-1-yl)-4-(trifluoromethyl)pyridin-2-yl)pyrrolidin-3-yl)propionic acid (26 mg, 0.068 mmol, yield: 20.0%). LCMS (ESI): [M+1]=383.4.

¹H NMR (400 MHz, CD₃OD) δ 6.12 (s, 1H), 4.73-4.58 (m, 1H), 4.42 (td, J=8.9, 5.1 Hz, 1H), 4.08 (dd, J=15.9, 8.8 Hz, 1H), 4.00-3.75 (m, 1H), 3.71-3.45 (m, 1H), 3.40 (dd, J=17.1, 9.5 Hz, 1H), 3.11-2.96 (m, 1H), 2.51-2.40 (m, 1H), 2.38-2.07 (m, 4H), 2.05-1.90 (m, 1H), 1.83-1.55 (m, 3H), 1.49 (d, J=6.2 Hz, 3H).

### Examples 22 and 23: Preparation of 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetic-2,2-d₂ acid and 2-((1R,5S,6R)-3-(5-cyano-6-((S)-2-methylazetidine-1-yl)-4-(trifluoromethyl)pyridin-2-yl-3-d)-3-azabicyclo[3.1.0]hexan-6-yl)acetic-2,2-d₂ acid

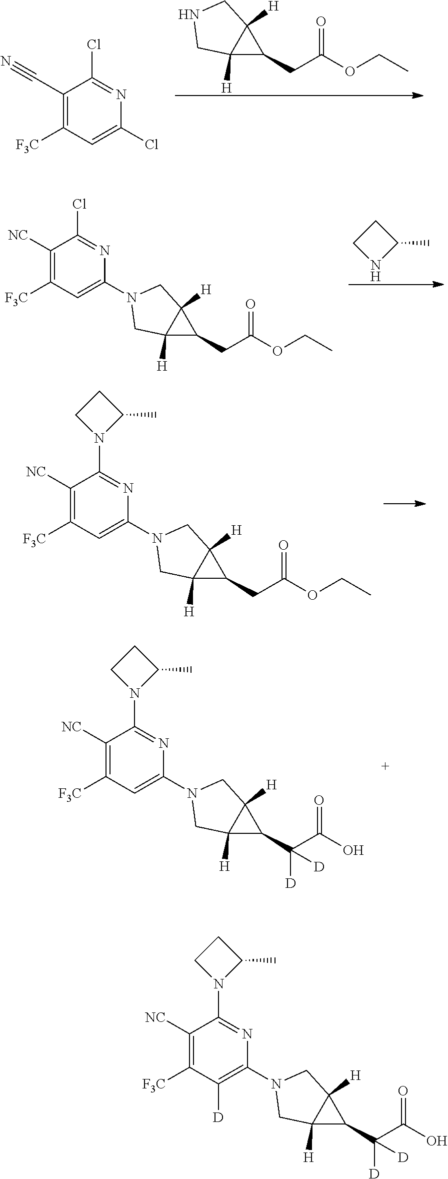

### Step 1: Synthesis of ethyl 2-((1R,5S,6s)-3-(6-chloro-5-cyano-4-(trifluoromethyl)pyridin-2-yl)-3-azabicyclo[3.1.0]hexan-6-yl)acetate

2,6-dichloro-4-(trifluoromethyl)nicotinonitrile (500 mg, 2.08 mmol) and 2-((1R,5S,6s)-3-azabicyclo[3.1.0]hexan-6-yl) ethyl acetate (386 mg, 2.28 mmol, 1.1 equiv.) was dissolved in DMF (10 mL); and potassium carbonate (860 mg, 6.23 mmol, 3.0 equiv.) was added. The reaction mixture was stirred for 3 hrs at room temperature. LCMS showed that the reaction was completed. Water (10 ml) was added to the reaction mixture, which was then extracted twice with ethyl acetate (15 ml), respectively. The organic phases were combined, washed twice with water (10 ml) and once with a saturated salt solution, then dried over anhydrous sodium sulfate, filtered, and spin-dried. The resulting crude product was separated by silica gel chromatographic columns to obtain ethyl 2-((1R,5S,6s)-3-(6-chloro-5-cyano-4-(trifluoromethyl)pyridin-2-yl)-3-azabicyclo[3.1.0]hexan-6-yl)acetate (747 mg, 2.00 mmol, yield: 96.3%).

¹H NMR (400 MHz, CDCl₃) δ 6.48 (s, 1H), 4.16 (q, J=7.1 Hz, 2H), 4.08-4.01 (m, 1H), 3.70-3.56 (m, 3H), 2.46-2.21 (m, 2H), 1.74-1.62 (m, 2H), 1.27 (t, J=7.1 Hz, 3H), 0.93 (tt, J=7.1, 3.5 Hz, 1H).

### Step 2: Synthesis of ethyl 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetate

Ethyl 2-((1R,5S,6s)-3-(6-chloro-5-cyano-4-(trifluoromethyl)pyridin-2-yl)-3-azabicyclo[3.1.0]hexan-6-yl)acetate (300 mg, 0.80 mmol) and (S)-2-methylazetidine (95 mg, 0.88 mmol, 1.1 equiv.) were dissolved in DMF (4 mL); and potassium carbonate (400 mg, 2.89 mmol, 3.6 equiv.) was added. The reaction mixture was stirred overnight at 80° C. LCMS showed that the reaction was completed. Water (10 ml) was added to the reaction mixture, which was then extracted twice with ethyl acetate (15 ml), respectively. The organic phases were combined, washed twice with water (10 ml) and once with a saturated salt solution, then dried over anhydrous sodium sulfate, filtered, and spin-dried. The resulting crude product was separated by silica gel chromatographic columns to obtain ethyl 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetate (315 mg, 0.771 mmol, yield: 96.0%). LCMS (ESI): [M+H]=409.4.

### Step 3: Synthesis of 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetic-2,2-d₂ acid and 2-((1R,5S,6R)-3-(5-cyano-6-((S)-2-methylazetidine-1-yl)-4-(trifluoromethyl)pyridin-2-yl-3-d)-3-azabicyclo[3.1.0]hexan-6-yl)acetic-2,2-d₂ acid

Ethyl 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetate (212 mg, 0.30 mmol) was dissolved in deuteriomethanol-d₄ (4 mL); and sodium methoxide (70 mg, 1.30 mmol, 4.3 equiv.) was added. The reaction occurred under the protection of nitrogen, and the reaction mixture was stirred for 3 days at room temperature. LCMS monitored that the reaction was completed. Acetic acid was added to adjust pH of the reaction mixture to weak acid, and the reaction mixture was spin-dried under reduced pressure. The resulting crude product was separated by reverse-phase preparation to obtain the mixture of 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetic-2,2-d₂ acid and 2-((1R,5S,6R)-3-(5-cyano-6-((S)-2-methylazetidine-1-yl)-4-(trifluoromethyl)pyridin-2-yl-3-d)-3-azabicyclo[3.1.0]hexan-6-yl)acetic-2,2-d₂ acid (1:1).

¹H NMR (400 MHz, CD₃OD) δ 6.09 (s, 0.5H), 4.67-4.62 (m, 1H), 4.45-4.39 (m, 1H), 4.11-4.05 (m, 1H), 3.51-3.48 (m, 1H), 2.47-2.43 (m, 1H), 2.01-1.96 (m, 1H), 1.62 (BR, 2H), 1.49 (d, 3H, J=0.8 Hz), 0.83-0.81 (m, 1H).

Or: ethyl 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetate (212 mg, 0.52 mmol, 1.0 equiv.) was dissolved in deuteriomethanol (4 mL); and sodium methoxide (70 mg, 1.30 mmol, 2.5 equiv.) was added. The reaction occurred under the protection of nitrogen, and the reaction mixture was stirred for 24 hrs at room temperature. Sodium methoxide (14 mg, 0.26 mmol, 0.5 equiv.) was added, and the reaction mixture was continuously stirred for 16 hrs. LCMS monitored that the reaction was completed. Acetic acid (94 mg, 3.0 equiv.) was added to adjust pH of the reaction mixture to weak acid, and the reaction mixture was spin-dried under reduced pressure. The resulting crude product was separated by reverse-phase preparation to obtain 2-((1R,5S,6R)-3-(5-cyano-6-((S)-2-methylazetidine-1-yl)-4-(trifluoromethyl)pyridin-2-yl)-3-azabicyclo[3.1.0]hexan-6-yl)acetic-2,2-d₂ acid (25.4 mg, 0.066 mmol, yield: 12.7%). LCMS (ESI): [M+H]⁺=383.2.

¹H NMR (400 MHz, CD₃OD) δ 6.09 (s, 1H), 4.65 (dd, J=14.2, 6.4 Hz, 1H), 4.42 (td, J=8.9, 5.1 Hz, 1H), 4.08 (dd, J=15.9, 8.9 Hz, 1H), 4.03-3.41 (m, 4H), 2.52-2.39 (m, 1H), 1.99 (ddt, J=10.9, 9.1, 6.8 Hz, 1H), 1.62 (s, 2H), 1.49 (d, J=6.2 Hz, 3H), 0.82 (t, J=3.2 Hz, 1H).

### Example 24: Preparation of (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylic acid

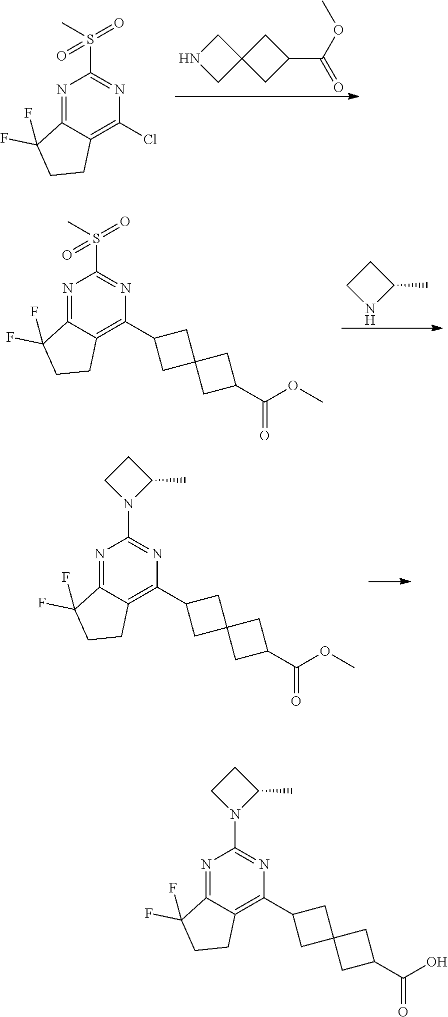

### Step 1: Synthesis of methyl 2-(7,7-difluoro-2-(methylsulfonyl)-6,7-dihydro-5H-cyclopentadiene[d]pyrimidin-4-yl)-2-azaspiro[3.3]heptan-6-carboxylate

4-chloro-7,7-difluoro-2-(methylsulfonyl)-6,7-dihydro-5H-cyclopenta[d]pyrimidine (1.0, 3.7 mmol, 1.0 equiv.) was dissolved in 7 ml of dry DMF; methyl 2-azaspiro[3.3]heptan-6-carboxylate (0.7 g, 4.5 mmol, 1.20 equiv.) and DIPEA (1.0 g, 7.4 mmol, 2.0 equiv.) were added; the reaction mixture was heated to 80° C., and stirred for 2 hrs, and LCMS showed that the reaction occurred completely; the reaction mixture was cooled to room temperature; water was added (15 ml) for quenching; EtOAc extraction was conducted twice (10 mL*2); the organic phases were combined, washed with a salt solution, dried over sodium sulfate, filtered, and concentrated; the resulting crude product was separated by silica gel column chromatography (PE/EtOAc=1:1) to obtain methyl 2-(7,7-difluoro-2-(methylsulfonyl)-6,7-dihydro-5H-cyclopentadiene[d]pyrimidin-4-yl)-2-azaspiro[3.3]heptan-6-carboxylate (1.0 g, yield: 69%). LCMS(ESI): [M+H]⁺=388.3.

### Step 2: Synthesis of methyl (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylate

In a microwave tube, methyl 2-(7,7-difluoro-2-(methylsulfonyl)-6,7-dihydro-5H-cyclopentadiene[d]pyrimidin-4-yl)-2-azaspiro[3.3]heptan-6-carboxylate (110 mg, 0.28 mmol, 1.0 equiv.) was dissolved in NMP (1 ml); triethylamine (66 mg, 0.93 mmol, 3.0 equiv.) and (S)-2-methylazetidine (36 mg, 0.35 mmol, 1.4 equiv.) were added; reaction was allowed to occur under microwaves, by which the reaction mixture was heated 160° C. to react for 6 hrs. LCMS showed that the occurrence of the reaction was substantially complete. The reaction mixture was cooled to room temperature and concentrated; the resulting crude product was separated by silica gel column chromatography (PE/EtOAc=2:1) to obtain methyl (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylate (60 mg, yield: 68%), LCMS(ESI): [M+H]=379.4.

### Step 3: Synthesis of (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylic acid

Methyl (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylate (60 mg, 0.16 mmol, 1.00 equiv.) was dissolved in a 2 ml mixed solvent of tetrahydrofuran and water (1:1, v/v); LiOH·H₂O (7.0 mg, 0.18 mmol, 1.1 equiv.) was added; and the resulting mixture was stirred overnight at room temperature for reaction. LCMS showed that the reaction occurred completely. 3 ml of water was added to dilute the mixture, which was then extracted twice with EtOAc (3 mL*2). 1N HCl was dropwise added to the resulting water solution to adjust the pH of the solution to 4; the resulting mixture was then extracted twice with EtOAc (10 mL×2); the organice phases were combined, washed with a salt solution, dried over sodium sulfate, filtered, and concentrated. The resulting crude product was separated by preparative HPLC to obtain (S)-2-(7,7-difluoro-2-(2-methylazetidine-1-yl)-6,7-dihydro-5H-cyclopentadiene[d]pyrimidin-4-yl)-2-azaspiro[3.3]heptan-6-carboxylic acid (20 mg, 34%). LCMS(ESI): [M−H]⁻=363.3, [M+H]⁺=365.4.

¹H NMR (400 MHz, DMSO-d₆) δ 12.10 (s, 1H), 4.32-4.00 (m, 4H), 3.87-3.75 (m, 1H), 2.94 (t, J=8.2 Hz, 1H), 2.78 (d, J=6.0 Hz, 2H), 2.45-2.28 (m, 6H), 2.20-1.97 (m, 1H), 1.87 (t, J=9.0 Hz, 1H), 1.53-1.33 (m, 4H), 0.85 (t, J=6.5 Hz, 1H).

Examples 25-36 may be prepared by selecting corresponding starting materials by referring completely or in part to the synthesis method for Example 24.

| Example | | | MS m/z |
|---|---|---|---|
| No. | Structural Formula | Chemical Name | [M + 1]⁺ |
| 25 | 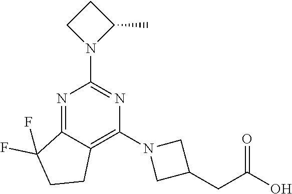 [SMILES: C[C@H]1CCN1c1nc(N2CC(CC(=O)O)C2)c2c(n1)C(F)(F)CC2] | (S)-2-(1-(7,7-difluoro-2-(2-methylazetidine- 1-yl)-6,7-dihydro-5H- cyclopentadiene[d]pyrimidin-4-yl) azetidine-3-yl)acetic acid | 339 |
| 26 | 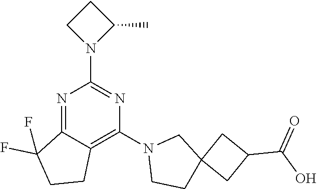 [SMILES: C[C@H]1CCN1c1nc(N2CCC3(CC(C(=O)O)C3)C2)c2c(n1)C(F)(F)CC2] | (S)-6-(7,7-difluoro-2-(2-methylazetidine- 1-yl)-6,7-dihydro-5H- cyclopentadiene[d]pyrimidin-4-yl)-6- azaspiro[3.4]octan-2-carboxylic acid | 379 |
| 27 | 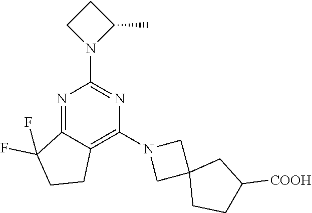 [SMILES: C[C@H]1CCN1c1nc(N2CC3(CCC(C(=O)O)C3)C2)c2c(n1)C(F)(F)CC2] | 2-(7,7-difluoro-2-((S)-2-methylazetidine- 1-yl)-6,7-dihydro-5H-cyclopentadiene[d] pyrimidin-4-yl)-2-azaspiro[3.4]octan-6- carboxylic acid | 379 |
| 28 | 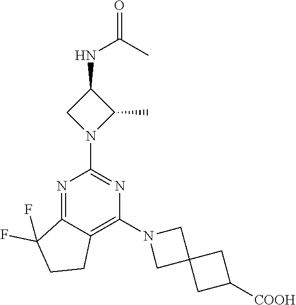 [SMILES: CC(=O)N[C@@H]1CN(c2nc(N3CC4(CC(C(=O)O)C4)C3)c3c(n2)C(F)(F)CC3)[C@H]1C] | 2-(2-((2S,3R)-3-acetamido-2- methylazetidine-1-yl)-7,7-difluoro-6,7- dihydro-5H-cyclopentadiene[d] pyrimidin-4-yl)-2-azaspiro[3.3] heptan-6-carboxylic acid | 422 |
| 29 | 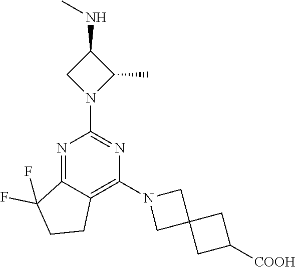 [SMILES: CN[C@@H]1CN(c2nc(N3CC4(CC(C(=O)O)C4)C3)c3c(n2)C(F)(F)CC3)[C@H]1C] | 2-(7,7-difluoro-2-((2S,3R)-2- methyl-3-(methylamino)azetidine-1-yl)- 6,7-dihydro-5H-cyclopentadiene [d]pyrimidin-4-yl)-2-azaspiro[3.3] heptan-6-carboxylic acid | 394 |
| 30 | 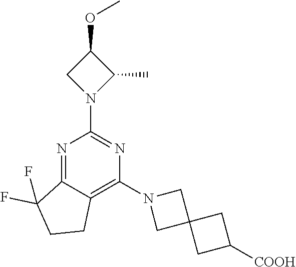 [SMILES: CO[C@@H]1CN(c2nc(N3CC4(CC(C(=O)O)C4)C3)c3c(n2)C(F)(F)CC3)[C@H]1C] | 2-(7,7-difluoro-2-((2S,3R)-3- methoxy-2-methylazetidine-1-yl)-6,7- dihydro-5H-cyclopentadiene[d] pyrimidin-4-yl)-2-azaspiro[3.3]heptan- 6-carboxylic acid | 395 |
| 31 | 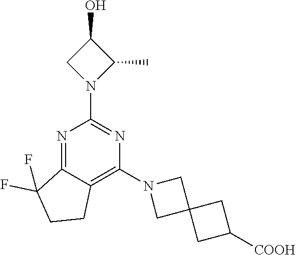 [SMILES: C[C@H]1[C@H](O)CN1c1nc(N2CC3(CC(C(=O)O)C3)C2)c2c(n1)C(F)(F)CC2] | 2-(7,7-difluoro-2-((2S,3R)-3-hydroxy- 2-methylazetidine-1-yl)-6,7-dihydro- 5H-cyclopentadiene[d]pyrimidin-4- yl)-2-azaspiro[3.3]heptan-6- carboxylic acid | 381 |
| 32 | 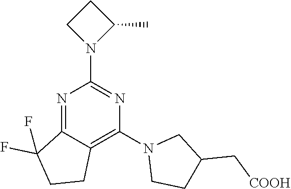 [SMILES: C[C@H]1CCN1c1nc(N2CCC(CC(=O)O)C2)c2c(n1)C(F)(F)CC2] | 2-(1-(7,7-difluoro-2-((S)-2- methylazetidine-1-yl)-6,7-dihydro-5H- cyclopentadiene[d]pyrimidin-4-yl) pyrrolidin-3-yl)acetic acid | 353 |
| 33 | 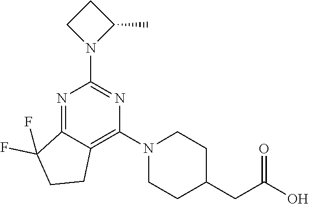 [SMILES: C[C@H]1CCN1c1nc(N2CCC(CC(=O)O)CC2)c2c(n1)C(F)(F)CC2] | (S)-2-(1-(7,7-difluoro-2-(2- methylazetidine-1-yl)-6,7-dihydro-5H- cyclopentadiene[d]pyrimidin-4-yl) piperidin-4-yl)acetic acid | 367 |
| 34 | 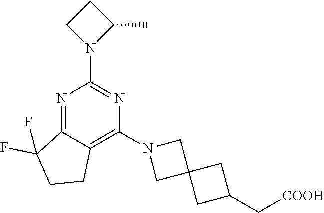 [SMILES: C[C@H]1CCN1c1nc(N2CC3(CC(CC(=O)O)C3)C2)c2c(n1)C(F)(F)CC2] | (S)-2-(2-(7,7-difluoro-2-(2- methylazetidine-1-yl)-6,7-dihydro-5H- cyclopentadiene[d]pyrimidin-4-yl)-2- azaspiro[3.3]heptan-6-yl)acetic acid | 379 |
| 35 | 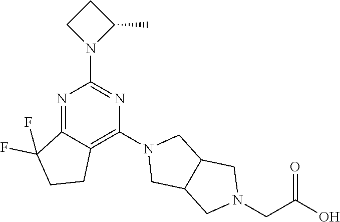 [SMILES: C[C@H]1CCN1c1nc(N2CC3CN(CC(=O)O)CC3C2)c2c(n1)C(F)(F)CC2] | 2-(5-(7,7-difluoro-2-((S)-2- methylazetidine-1-yl)-6,7-dihydro-5H- cyclopentadiene[c]pyrimidin-4-yl) hexahydropyrrolo[3,4-c]pyrrolo- 2(1H)-yl)acetic acid | 394 |
| 36 | 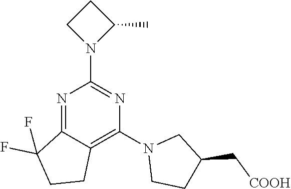 [SMILES: C[C@H]1CCN1c1nc(N2CC[C@H](CC(=O)O)C2)c2c(n1)C(F)(F)CC2] | 2-((R)-1-(7,7-difluoro-2-((S)-2- methylazetidine-1-yl)-6,7-dihydro-5H- cyclopentadiene[d]pyrimidin-4- yl)pyrrolidin-3-yl)acetic acid | 353 |

### Example 37: Preparation of 2-((R)-1-(7,7-difluoro-2-((S)-2-methylazetidine-1-yl)-6,7-dihydro-5H-cyclopentadiene[d]pyrimidin-4-yl)pyrrolidin-3-yl)-N-(2-hydroxyethyl)acetamide

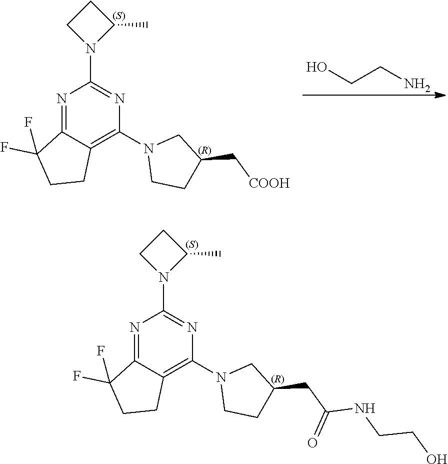

2-((R)-1-(7,7-difluoro-2-((S)-2-methylazetidine-1-yl)-6,7-dihydro-5H-cyclopentadiene[d]pyrimidin-4-yl)pyrrolidin-3-yl)acetic acid (70.0 mg, 0.20 mmol) and aminoethanol (36.0 mg, 0.60 mmol, 3.00 equiv.) were dissolved in DMF (1 mL); and HATU (90.0 mg, 0.24 mmol, 1.50 equiv.) and DIPEA (0.16 mL, 1.00 mmol, 5.00 equiv.) were added. The resulting mixture was allowed to react overnight at room temperature under the protection of nitrogen. The reaction mixture was quenched with water (10 mL), and extracted with EtOAc (20 mL*3); and the organice phases were washed with a saturated salt solution (20 mL*3), dried over anhydrous Na₂SO₄, filtered, and spin-dried. Separation was conducted by reverse-phase preparation to obtain 2-((R)-1-(7,7-difluoro-2-((S)-2-methylazetidine-1-yl)-6,7-dihydro-5H-cyclopentadiene[d]pyrimidin-4-yl)pyrrolidin-3-yl)-N-(2-hydroxyethyl)acetamide (25.90 mg, 0.12 mmol, 32.9%). LCMS(ESI): [M+1]⁺=396.4.

¹H NMR (400 MHz, CD₃OD) δ 4.42 (dd, J=14.1, 6.3 Hz, 1H), 4.04-3.99 (m, 1H), 3.89 (dd, J=16.2, 8.8 Hz, 3H), 3.67 (s, 1H), 3.61 (t, J=5.8 Hz, 2H), 3.33 (s, 3H), 3.10 (d, J=3.8 Hz, 2H), 2.63 (s, 1H), 2.47-2.34 (m, 5H), 2.13 (s, 1H), 1.95 (dd, J=17.6, 8.9 Hz, 1H), 1.69 (s, 1H), 1.49 (d, J=6.2 Hz, 3H).

### Embodiment 38: Preparation of 2-((R)-1-(7,7-difluoro-2-((S)-2-methylazetidine-1-yl)-6,7-dihydro-5H-cyclopentadiene[d]pyrimidin-4-yl)pyrrolidin-3-yl)-N-hydroxyacetamide

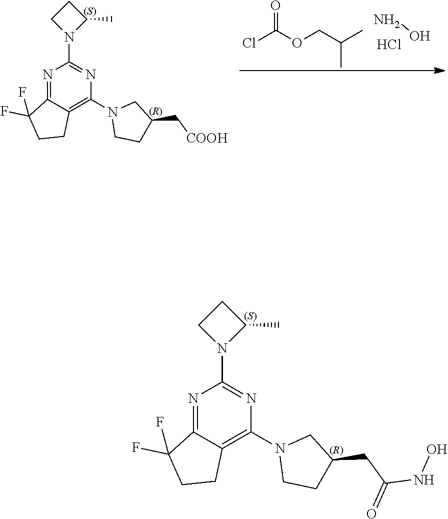

Hydroxylamine hydrochloride (200.0 mg, 2.88 mmol) was dissolved in MeOH (3 mL); KOH (161.0 mg, 2.88 mmol, 1.00 equiv.) was added; and the resulting mixture was stirred for 1 hour under an ice-water bath. The reaction mixture was filtered to remove salt to obtain the solution of free-state NH₂OH in methanol.

A compound isobutyl chlorocarbonate (30.0 mg, 0.30 mmol, 1.50 equiv.) and triethylamine (41.0 mg, 0.30 mmol, 1.50 equiv.) were added to the solution of 2-((R)-1-(7,7-difluoro-2-((S)-2-methylazetidine-1-yl)-6,7-dihydro-5H-cyclopentadiene[d]pyrimidin-4-yl)pyrrolidin-3-yl)acetic acid (70.0 mg, 0.20 mmol, 1.00 equiv.) in THF (2 mL), and the resulting mixture was stirred for 1 hour at room temperature under the protection of nitrogen. The prepared free NH₂OH (52.0 mg, 1.6 mmol, 8.00 equiv.) was added to the reaction mixture to react for 3 hrs at room temperature. The reaction mixture was concentrated under reduced pressure to obtain an oily product, which was separated by reverse-phase preparation to obtain 2-((R)-1-(7,7-difluoro-2-((S)-2-methylazetidine-1-yl)-6,7-dihydro-5H-cyclopentadiene[d]pyrimidin-4-yl)pyrrolidin-3-yl)-N-hydroxyacetamide (24.2 mg, 0.12 mmol, 33.2%). LCMS(ESI): [M+1]⁺=368.0.

¹H NMR (400 MHz, CD₃OD) δ 4.47-4.38 (m, 1H), 4.05-3.99 (m, 1H), 3.95-3.78 (m, 3H), 3.69 (s, 1H), 3.39 (s, 1H), 3.10 (dd, J=7.4, 3.7 Hz, 2H), 2.67-2.59 (m, 1H), 2.49-2.34 (m, 3H), 2.23 (dd, J=7.4, 1.4 Hz, 2H), 2.14 (d, J=6.0 Hz, 1H), 1.98-1.89 (m, 1H), 1.69 (s, 1H), 1.48 (d, J=6.2 Hz, 3H).

Example 39 may be prepared by selecting corresponding starting materials by referring completely or in part to the synthesis method for Example 38.

| Example | | | MS m/z |
|---|---|---|---|
| No. | Structural Formula | Chemical Name | [M + 1]⁺ |
| 39 | 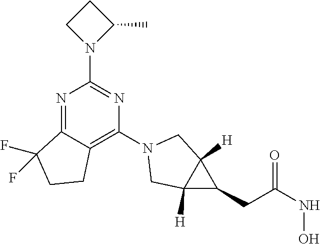 [SMILES: C[C@H]1CCN1c1nc(N2C[C@@H]3[C@@H](CC(=O)NO)[C@@H]3C2)c2c(n1)C(F)(F)CC2] | 2-((1R,5S,6R)-3-(7,7-difluoro-2- ((S)-2-methylazetidine-1-yl)-6,7- dihydro-5H-cyclopentadiene[d] pyrimidin-4-yl)-3-azabicyclo[3.1.0] hexan-6-yl)-N-hydroxyacetamide | 380 |

### Embodiment 40: Preparation of 2-((1R,5S,6R)-3-(5-((S)-2-methylazetidine-1-yl)pyridino[3,4-b]pyrazin-7-yl)-3-azabicyclo[3.1.0]hexan-6-yl)acetic acid

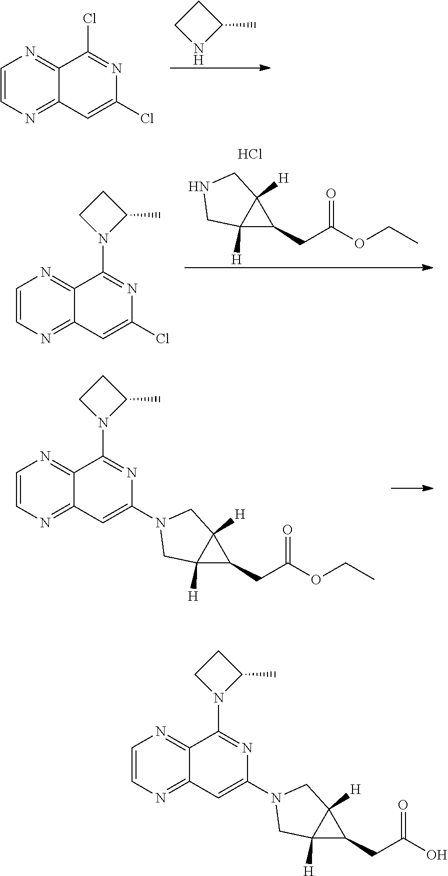

### Step 1: Synthesis of(S)-7-chloro-5-(2-methylazetidine-1-yl)pyridino[3,4-b]pyrazine

(S)-2-methylazetidine (54 mg, 0.50 mmol, 1.00 equiv.) and DIPEA (0.22 mL, 1.25 mmol, 2.5 equiv.) were added to the solution of 5,7-dichloropyridino[3,4-b]pyrazine (100 mg, 0.50 mmol) in NMP (1 mL), and the resulting mixture was treated with microwaves at 130° C. to react for 0.5 hour. The reaction mixture was quenched with water (10 mL), and extracted with EtOAc (20 mL*3). The organic phases were washed with saturated salt solutions (20 mL*3), dried over anhydrous Na₂SO₄, filtered, and spin-dried. The resulting crude product was separated by the preparation plate (PE:EA=2:1) to obtain (S)-7-chloro-5-(2-methylazetidine-1-yl)pyridino[3,4-b]pyrazine (90 mg, yield: 76.71%).

### Step 2: Synthesis of ethyl 2-((1R,5S,6R)-3-(5-((S)-2-methylazetidine-1-yl)pyridino[3,4-b]pyrazin-7-yl)-3-azabicyclo[3.1.0]hexan-6-yl)ethyl acetate

Pd₂(dba)₃ (87 mg, 0.95 mmol, 0.20 equiv.), XantPhos (83 mg, 0.14 mmol, 0.30 equiv.), and Cs₂CO₃ (311 mg, 0.95 mmol, 2.00 equiv.) were added to the solution of (S)-7-chloro-5-(2-methylazetidine-1-yl)pyridino[3,4-b]pyrazine (112 mg, 0.48 mmol) and 2-((1R,5S,6s)-3-azabicyclo[3.1.0]hexan-6-yl)ethyl acetate hydrochloride (121 mg, 0.72 mmol, 1.50 equiv.) in dioxane (3 mL). The resulting mixture was allowed to react overnight at 110° C. under the protection of nitrogen. The reaction mixture was quenched with water (10 mL), and extracted with EA (20 mL*3). The organic phases were washed with a saturated salt solution (20 mL), dried over anhydrous Na₂SO₄, filtered, and spin-dried. The resulting crude product was separated by the preparation plate (PE:EA=2:1) to obtain ethyl 2-((1R,5S,6R)-3-(5-((S)-2-methylazetidine-1-yl)pyridino[3,4-b]pyrazin-7-yl)-3-azabicyclo[3.1.0]hexan-6-yl) ethyl acetate (90 mg, yield: 61.59%).

### Step 3: Synthesis of 2-((1R,5S,6R)-3-(5-((S)-2-methylazetidine-1-yl)pyridino[3,4-b]pyrazin-7-yl)-3-azabicyclo[3.1.0]hexan-6-yl)acetic acid

LiOH H₂O (25 mg, 0.59 mmol, 2.00 equiv.) was added to the mixed solution of 2-((1R,5S,6R)-3-(5-((S)-2-methylazetidine-1-yl)pyridino[3,4-b]pyrazin-7-yl)-3-azabicyclo[3.1.0]hexan-6-yl) ethyl acetate (108 mg, 0.29 mmol) in THF (3 mL) and water (0.5 mL). The mixture reaction was react overnight at room temperature under the protection of nitrogen. The reaction mixture was quenched with water (20 mL), and extracted with DCM (20 mL*3). The aqueous phase was acidized with dilute hydrochloric acid to achieve pH=5, and extracted with EA (30 mL*3). The organic phases were dried over anhydrous Na₂SO₄, filtered, and spin-dried. The resulting crude product was separated by neutral reverse-phase preparation to obtain 2-((1R,5S,6R)-3-(5-((S)-2-methylazetidine-1-yl)pyridino[3,4-b]pyrazin-7-yl)-3-azabicyclo[3.1.0]hexan-6-yl)acetic acid (27.9 mg, yield: 28.02%).

¹H NMR (400 MHz, CD₃OD) δ 8.35 (d, J=2.1 Hz, 1H), 8.13 (d, J=2.0 Hz, 1H), 4.81-4.73 (m, 1H), 4.64-4.54 (m, 1H), 4.41-4.29 (m, 1H), 3.80 (dd, J=24.6, 10.3 Hz, 2H), 3.45 (dd, J=20.1, 8.1 Hz, 2H), 2.61-2.49 (m, 1H), 2.31 (d, J=7.2 Hz, 2H), 2.10-1.94 (m, 1H), 1.61-1.56 (m, 5H), 0.95-0.86 (m, 1H).

Examples 41-45 may be prepared by selecting corresponding starting materials by referring completely or in part to the synthesis method for Example 40.

| Example | | | MS m/z |
|---|---|---|---|
| No. | Structural Formula | Chemical Name | [M + 1]⁺ |
| 41 | 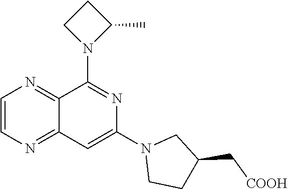 [SMILES: C[C@H]1CCN1c1nc(N2CC[C@H](CC(=O)O)C2)cc2nccnc12] | 2-((R)-1-(5-((S)-2-methylazetidine- 1-yl)pyridino[3,4-b]pyrazin-7-yl) pyrrolidin-3-yl)acetic acid | 328 |
| 42 | 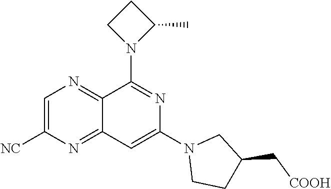 [SMILES: C[C@H]1CCN1c1nc(N2CC[C@H](CC(=O)O)C2)cc2nc(C#N)cnc12] | 2-((R)-1-(2-cyano-5-((S)-2- methylazetidine-1-yl)pyridino[3,4-b] pyrazin-7-yl)pyrrolidin-3-yl)acetic acid | 353 |
| 43 | 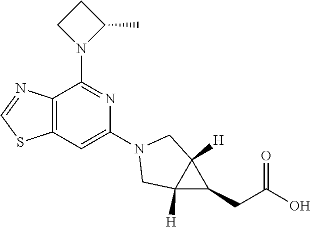 [SMILES: C[C@H]1CCN1c1nc(N2C[C@@H]3[C@@H](CC(=O)O)[C@@H]3C2)cc2scnc12] | 2-((1R,5S,6R)-3-(4-((S)-2-methyl- azetidine-1-yl)thiazolo[4,5-c] pyridin-6-yl)-3-azabicyclo[3.1.0] hexan-6-yl)acetic acid | 345 |
| 44 | 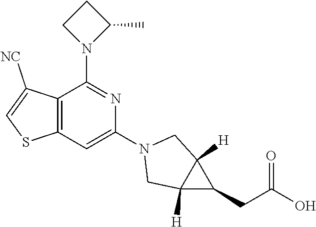 [SMILES: C[C@H]1CCN1c1nc(N2C[C@@H]3[C@@H](CC(=O)O)[C@@H]3C2)cc2scc(C#N)c12] | 2-((1R,5S,6R)-3-(3-cyano-4-((S)- 2-methylazetidine-1-yl)thieno[3,2- c]pyridin-6-yl)-3-azabicyclo [3.1.0]hexan-6-yl)acetic acid | 369 |
| 45 | 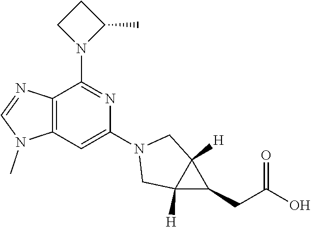 [SMILES: C[C@H]1CCN1c1nc(N2C[C@@H]3[C@@H](CC(=O)O)[C@@H]3C2)cc2c1ncn2C] | 2-((1R,5S,6R)-3-(1-methyl-4-((S)- 2-methylazetidine-1-yl)-1H- imidazo[4,5-c]pyridin-6-yl)-3- azabicyclo[3.1.0]hexan-6-yl) acetic acid | 342 |

### Example 46: Preparation of 2-((1R,5S,6R)-3-(2-cyano-5-((S)-2-methylazetidine-1-yl)pyridino[3,4-b]pyrazin-7-yl)-3-azabicyclo[3.1.0]hexan-6-yl)acetic acid

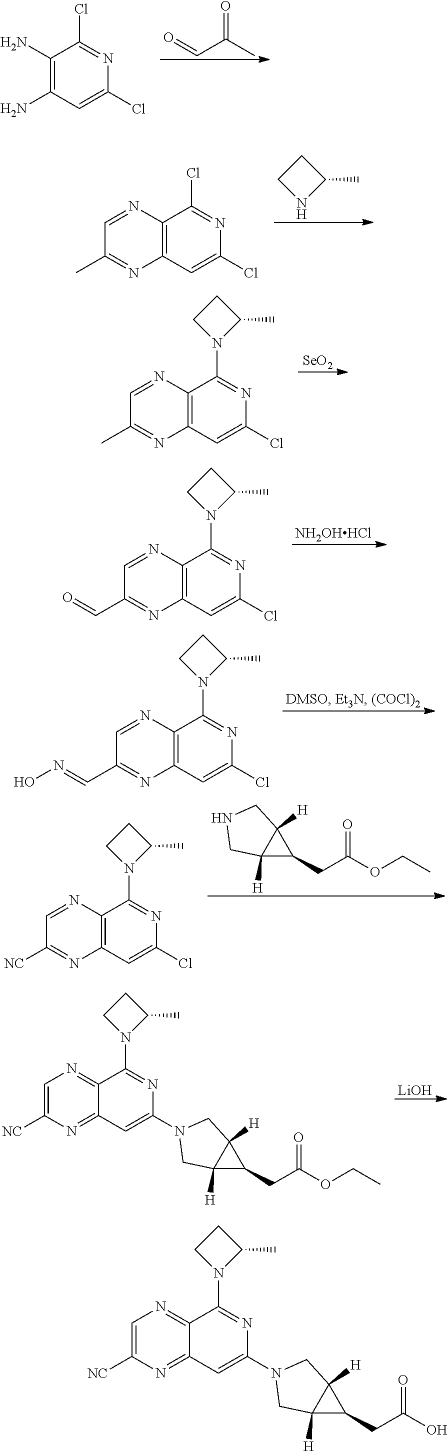

### Step 1: Synthesis of 5,7-dichloro-2-methylpyridino[3,4-b]pyrazine

2,6-dichloropyridin-3,4-diamine (500.0 mg, 2.81 mmol) and 2-oxopropionaldehyde (404.8 mg, 5.62 mmol) was dissolved in deionized water (10 mL); sodium hydrogen sulfite (292.3 mg, 2.81 mmol) was added; the resulting mixture was stirred for 4 hrs at 80° C.; and the reaction mixture turned into brownish red. TLC (petroleum ether/ethyl acetate=3/1) showed that one man point was produced. The reaction mixture was diluted with 50 mL of water, and then extracted with ethyl acetate (30 mL*3); the organic phases were dried over anhydrous sodium sulfate, filtered, concentrated under reduced pressure, and finally purified by rapid column chromatography (petroleum ether/ethyl acetate=3/1, Rf=0.5) to obtain 5,7-dichloro-2-methylpyridino[3,4-b]pyrazine (300.0 mg, 1.4 mmol, yield: 49.9%).

¹H NMR (400 MHz, CDCl₃) δ 8.87 (s, 1H), 7.86 (s, 1H), 2.85 (s, 3H).

### Step 2: Synthesis of (S)-7-chloro-2-methyl-5-(2-methylazetidine-1-yl)pyridino[3,4-b]pyrazine

5,7-dichloro-2-methylpyridino[3,4-b]pyrazine (300.0 mg, 1.4 mmol) and (S)-2-methylazetidine (109.6 mg, 1.54 mmol) were dissolved in 1-methylpyrrolidone (5 mL); diisopropylethylamine (543.4 mg, 4.2 mmol) was added. The resulting mixture was heated by microwaves to 130° C. and stirred for 1 hour, and TLC (petroleum ether/ethyl acetate=5/1) showed that the reaction between the starting materials was completed, with a main point produced. The reaction mixture was diluted with 30 mL of water, and then extracted with ethyl acetate (30 mL*3); the organic phases were dried over anhydrous sodium sulfate, filtered, concentrated under reduced pressure, and finally purified by rapid column chromatography (petroleum ether/ethyl acetate=5/1, Rf=0.5) to obtain (S)-7-chloro-2-methyl-5-(2-methylazetidine-1-yl)pyridino[3,4-b]pyrazine (300.0 mg, 1.21 mmol, yield: 86.1%).

¹H NMR (400 MHz, CDCl₃) δ 8.44 (s, 1H), 6.94 (s, 1H), 5.04-4.90 (m, 1H), 4.71-4.57 (m, 1H), 4.48-4.35 (m, 1H), 2.68 (s, 3H), 2.66-2.60 (m, 1H), 2.09-2.02 (m, 1H), 1.61 (d, J=6.4 Hz, 3H).

### Step 3: Synthesis of (S)-7-chloro-5-(2-methylazetidine-1-yl)pyridino[3,4-b]pyrazin-2-formaldehyde

(S)-7-chloro-2-methyl-5-(2-methylazetidine-1-yl)pyridino[3,4-b]pyrazine (300.0 mg, 1.21 mmol) was dissolved in dioxane (5 mL); and selenium dioxide (200.7 mg, 1.81 mmol) was added. The reaction mixture was heated to 80° C. and stirred for 10 hrs, and TLC (petroleum ether/ethyl acetate=5/1) showed that the reaction between the starting materials was completed, with a new point produced. The reaction mixture was diluted with 20 mL of water, and then extracted with ethyl acetate (20 mL*3); the organic phases were dried over anhydrous sodium sulfate, filtered, concentrated under reduced pressure, and finally purified by rapid column chromatography (petroleum ether/ethyl acetate=7/1, Rf=0.4) to obtain (S)-7-chloro-5-(2-methylazetidine-1-yl)pyridino[3,4-b]pyrazin-2-formaldehyde (150.0 mg, 0.57 mmol, yield: 47.3%).

¹H NMR (400 MHz, CDCl₃) δ 10.16 (s, 1H), 9.07 (s, 1H), 7.08 (s, 1H), 5.12-4.90 (m, 1H), 4.78-4.60 (m, 1H), 4.56-4.37 (m, 1H), 2.73-2.64 (m, 1H), 2.13-2.05 (m, 1H), 1.63 (d, J=6.4 Hz, 3H).

### Step 4: Synthesis of (S,E)-7-chloro-5-(2-methylazetidine-1-yl)pyridino[3,4-b]pyrazin-2-formaldoxime

(S)-7-chloro-5-(2-methylazetidine-1-yl)pyridino[3,4-b]pyrazin-2-formaldehyde (150.0 mg, 0.57 mmol) was dissolved in anhydrous ethanol (5 mL); and hydroxylamine hydrochloride (119.0 mg, 1.71 mmol) was added. The reaction mixture was stirred for 12 hrs at room temperature, and LCMS showed that the reaction was completed. The reaction mixture was diluted with 20 mL of water, and then extracted with ethyl acetate (20 mL*3); the organic phases were dried over anhydrous sodium sulfate, filtered, concentrated under reduced pressure to obtain (S,E)-7-chloro-5-(2-methylazetidine-1-yl)pyridino[3,4-b]pyrazin-2-formaldoxime (120.0 mg, 0.43 mmol, yield: 75.7%). LCMS(ESI): [M+1]⁺=278.2. tR=1.273 min

### Step 5: Synthesis of (S)-7-chloro-5-(2-methylazetidine-1-yl)pyridino[3,4-b]pyrazin-2-nitrile

(S,E)-7-chloro-5-(2-methylazetidine-1-yl)pyridino[3,4-b]pyrazin-2-formaldoxime (120.0 mg, 0.43 mmol) was dissolved in acetonitrile (5 mL); dimethyl sulfoxide (one drop) and triethylamine (131.2 mg, 1.30 mmol, 3.00 equiv) were added; the reaction mixture was cooled to 0° C.; then, oxalyl chloride (82.3 mg, 0.65 mmol, 1.50 equiv) was dropwise added; the reaction mixture was slowly heated to room temperature and then stirred for 12 hrs; and TLC (petroleum ether/ethyl acetate=3/1) showed that the reaction between the starting materials was completed, with a new point produced. The reaction mixture was diluted with 20 mL of water, and then extracted with ethyl acetate (20 mL*3); the organic phases were dried over anhydrous sodium sulfate, filtered, concentrated under reduced pressure, and finally purified by rapid column chromatography (petroleum ether/ethyl acetate=3/1, Rf=0.5) to obtain (S)-7-chloro-5-(2-methylazetidine-1-yl)pyridino[3,4-b]pyrazin-2-nitrile (50.0 mg, 0.19 mmol, yield: 44.6%).

¹H NMR (400 MHz, CDCl₃) δ 8.74 (s, 1H), 6.99 (s, 1H), 5.09-4.88 (m, 1H), 4.82-4.62 (m, 1H), 4.57-4.35 (m, 1H), 2.75-2.64 (m, 1H), 2.15-2.04 (m, 1H), 1.64 (d, J=6.4 Hz, 3H).

### Step 6: Synthesis of 2-((1R,5S,6R)-3-(2-cyano-5-((S)-2-methylazetidine-1-yl)pyridino[3,4-b]pyrazin-7-yl)-3-azabicyclo[3.1.0]hexan-6-yl) ethyl acetate

(S)-7-chloro-5-(2-methylazetidine-1-yl)pyridino[3,4-b]pyrazin-2-nitrile (50.0 mg, 0.19 mmol), 2-((1R,5S,6S)-3-azabicyclo[3.1.0]hexan-6-yl) ethyl acetate (48.9 mg, 0.29 mmol, 1.5 equiv), 4,5-bis(diphenylphosphino)-9,9-dimethylxanthene (22.3 mg, 38.51 umol, 0.2 equiv), tris(dibenzylideneacetone)dipalladium (35.3 mg, 38.51 umol, 0.2 equiv), and cesium carbonate (188.2 mg, 0.58 mmol, 3.00 equiv) were dissolved in anhydrous dioxane (2 mL), heated to 100° C., and stirred for 12 hrs, with the LCMS showing that the reaction was completed. The reaction mixture was diluted with 20 mL of water, and then extracted with ethyl acetate (20 mL*3); the organic phases were dried over anhydrous sodium sulfate, filtered, and concentrated under reduced pressure. The resulting crude product was purified using a large preparation plate (petroleum ether/ethyl acetate=3/1, Rf=0.3) to obtain 2-((1R,5S,6R)-3-(2-cyano-5-((S)-2-methylazetidine-1-yl)pyridino[3,4-b]pyrazin-7-yl)-3-azabicyclo[3.1.0]hexan-6-yl) ethyl acetate (10.0 mg, 15.29 umol, purity: 60%, yield: 7.9%). LCMS (ESI): [M+1]=393.3. tR=2.300 min.

### Step 7: Synthesis of 2-((1R,5S,6R)-3-(2-cyano-5-((S)-2-methylazetidine-1-yl)pyridino[3,4-b]pyrazin-7-yl)-3-azabicyclo[3.1.0]hexan-6-yl)acetic acid

2-((1R,5S,6R)-3-(2-cyano-5-((S)-2-methylazetidine-1-yl)pyridino[3,4-b]pyrazin-7-yl)-3-azabicyclo[3.1.0]hexan-6-yl) ethyl acetate (10.0 mg, 25.48 umol, 60% purity) was dissolved in methanol (2 mL) and water (0.5 mL); lithium hydroxide (3.0 mg, 127.40 umol, 5.00 equiv) was added; and the resulting mixture was stirred for 6 hrs at room temperature. LCMS detected that the reaction between the starting materials was completed. The reaction mixture was concentrated under reduced pressure to obtain an oily product, which was purified by reverse-phase preparation to obtain 2-((1R,5S,6R)-3-(2-cyano-5-((S)-2-methylazetidine-1-yl)pyridino[3,4-b]pyrazin-7-yl)-3-azabicyclo[3.1.0]hexan-6-yl)acetic acid (0.7 mg, 1.92 umol, yield: 7.5%). LCMS (ESI): [M+1]=365.1. tR=1.290 min.

¹H NMR (400 MHz, CD₃OD) δ 8.23 (s, 1H), 7.05 (s, 1H), 5.33-5.20 (m, 1H), 5.18-5.16 (m, 1H), 4.59-4.57 (m, 1H), 3.99-3.94 (m, 2H), 3.67-3.62 (m, 2H), 2.30-2.28 (m, 1H), 2.21-2.18 (m, 2H), 2.06-2.04 (m, 1H), 1.71-1.66 (m, 2H), 1.55 (d, J=6.4 Hz, 3H), 0.94-0.91 (m, 1H).

### Example 47: Preparation of 2-((1R,5S,6R)-3-(2-aminoformyl-5-((S)-2-methylazetidine-1-yl)pyridino[3,4-b]pyrazin-7-yl)-3-azabicyclo[3.1.0]hexan-6-yl)acetic acid

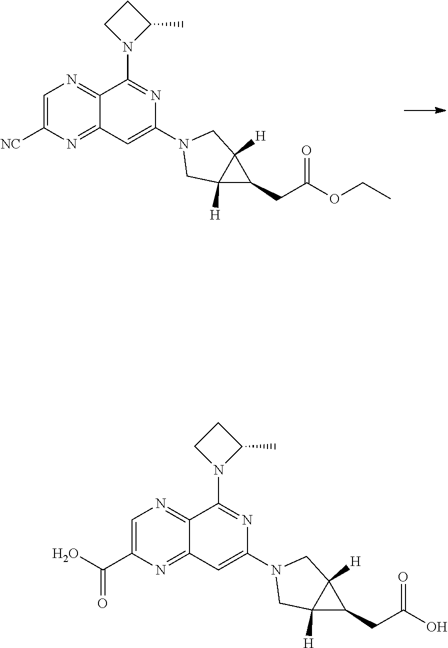 [SMILES: CCOC(=O)C[C@@H]1[C@H]2CN(c3cc4nc(C#N)cnc4c(N4CC[C@@H]4C)n3)C[C@@H]12 ; C[C@H]1CCN1c1nc(N2C[C@@H]3[C@@H](CC(=O)O)[C@@H]3C2)cc2nc(C(=O)O)cnc12]

2-((1R,5S,6R)-3-(2-cyano-5-((S)-2-methylazetidine-1-yl)pyridino[3,4-b]pyrazin-7-yl)-3-azabicyclo[3.1.0]hexan-6-yl) ethyl acetate (3.0 mg, 0.07 mmol, 1.00 equiv) was dissolved in tetrahydrofuran (2 mL) and water (0.5 mL); and lithium hydroxide monohydrate (16.0 mg, 0.38 mmol, 5.0 equiv) was added. The reaction mixture was heated and stirred for 30 hrs. LCMS detected that the reaction was completed. The reaction mixture was concentrated under reduced pressure; and the resulting crude product was separated by high-performance liquid chromatography prepared with a trifluoroacetic acid system, and freeze-dried to obtain 2-((1R,5S,6R)-3-(2-aminoformyl-5-((S)-2-methylazetidine-1-yl)pyridino[3,4-b]pyrazin-7-yl)-3-azabicyclo[3.1.0]hexan-6-yl)acetic acid (17.6 mg, yield: 45.1%). LCMS (ESI): [M+1 0.1=383.3, tR=1.385 min;

¹H NMR (400 MH z, DMSO-d₆) δ 11.77 (s, 1H), 8.65 (s, 1H), 8.13 (s, 1H), 7.80 (s, 1H), 5.82 (s, 1H), 4.73 (s, 1H), 4.54 (s, 1H), 4.32 (s, 1H), 3.82-3.60 (m, 4H), 2.52 (s, 1H), 2.30-2.21 (m, 2H), 2.05-1.96 (m, 1H), 1.59 (s, 2H), 1.53 (d, J=6.2 Hz, 3H), 0.84-0.73 (in, 1H).

The nuclear magnetic resonance data of the compounds prepared in the above examples were as follows:

| Example | |
|---|---|
| No. | ¹H NMR |
| 2 | ¹H NMR (400 MHz, CD₃OD) δ 5.980 (s, 1H), 4.62-4.67 (m, 1H), 4.39-4.45 (m, |
| | 1H), 4.01-4.12 (m, 1H), 3.70-3.83 (m, 4H), 2.43-2.48 (m, 1H), 2.27-2.30 (m, 1H), |
| | 1.89-2.00 (m, 5H), 1.51-1.64 (m, 4H), 1.48 (d, J = 6.4 Hz, 3H). |
| 3 | ¹H NMR (400 MHz, CD₃OD) δ 6.416 (s, 1H), 4.61-4.66 (m, 1H), 4.39-4.48 (m, |
| | 1H), 4.05-4.11 (m, 1H), 3.60-3.61 (m, 4H), 2.95-2.99 (m, 1H), 2.45-2.49 (m, 1H), |
| | 1.96-2.06 (m, 5H), 1.57-1.67 (m, 4H), 1.48 (d, J = 6 Hz, 3H). |
| 4 | ¹H NMR (400 MHz, DMSO-d₆) δ 6.14 (s, 1H), 4.62-4.53 (m, 1H), 4.47 (d, J = |
| | 18.3 Hz, 4H), 4.46-4.22 (m, 5H), 4.13-4.03 (m, 1H), 2.47-2.36 (m, 1H), |
| | 2.02-1.86 (m, 1H), 1.44 (d, J = 6.2 Hz, 3H). |
| | ¹⁹F NMR (377 MHz, DMSO-d₆) δ −63.62 (s, 3F). |
| 6 | ¹H NMR (400 MHz, DMSO-d₆) δ 6.18 (s, 1H), 4.60 (s, 1H), 4.33 (s, 1H), 4.07 |
| | (d, J = 8.4 Hz, 1H), 3.46 (d, J = 54.6 Hz, 4H), 3.07 (s, 1H), 2.41 (s, 1H), |
| | 2.22-2.12 (m, 4H), 1.96 (d, J = 7.9 Hz, 3H), 1.45 (s, 3H). |
| 7 | ¹H NMR (400 MHz, DMSO-d₆) δ 12.09 (s, 1H), 6.04 (s, 1H), 4.57 (dd, J = 13.9, |
| | 6.4 Hz, 1H), 4.42-4.23 (m, 1H), 4.07 (dd, J = 15.8, 8.6 Hz, 1H), 4.01-3.82 (m, |
| | 4H), 2.79 (d, J = 7.4 Hz, 1H), 2.40 (dd, J = 16.0, 7.9 Hz, 1H), 2.11 (dd, J = 13.0, |
| | 8.8 Hz, 1H), 2.01 (dd, J = 13.2, 7.3 Hz, 1H), 1.96-1.82 (m, 4H), 1.81-1.73 (m, |
| | 1H), 1.42 (d, J = 6.1 Hz, 3H). |
| 8 | ¹H NMR (400 MHz, DMSO-d₆) δ 12.14 (s, 1H), 6.18 (s, 1H), 4.58 (dd, J = 13.5, |
| | 6.5 Hz, 1H), 4.32 (dd, J = 13.5, 8.6 Hz, 1H), 4.06 (dd, J = 15.9, 8.5 Hz, 2H), |
| | 3.93-3.46 (m, 7H), 2.40 (s, 2H), 2.23 (d, J = 6.4 Hz, 3H), 1.94 (dt, J = 16.8, 8.5 |
| | Hz, 2H), 1.60 (s, 3H), 1.43 (d, J = 6.1 Hz, 5H), 0.74 (s, 2H). |
| 9 | ¹H NMR (400 MHz, CD₃OD) δ 5.94 (s, 1H), 4.68-4.61 (m, 1H), 4.42 (td, J = 8.8, |
| | 5.2 Hz, 1H), 4.16-4.05 (m, 3H), 4.02-3.94 (m, 2H), 2.61-2.53 (m, 1H), 2.51-2.38 |
| | (m, 5H), 2.04-1.95 (m, 3H), 1.48 (d, J = 6.4 Hz, 3H). |
| 10 | ¹H NMR (400 MHz, CDCl₃) δ 5.93 (s, 1H), 4.70-4.56 (m, 1H), 4.50 (td, J = 9.0, |
| | 5.2 Hz, 1H), 4.14 (dd, J = 16.0, 9.0 Hz, 1H), 4.02-3.38 (m, 4H), 2.52-2.33 (m, |
| | 1H), 2.07-1.88 (m, 2H), 1.74 (brs, 1H), 1.58 (brs, 1H), 1.49 (d, J = 6.2 Hz, 3H), |
| | 1.29 (d, J = 7.0 Hz, 3H), 0.84 (dt, J = 9.4, 3.3 Hz, 1H). |
| 11 | ¹H NMR (400 MHz, CDCl₃) δ 5.96 (s, 1H), 4.71-4.58 (m, 1H), 4.51 (td, J = 8.8, |
| | 5.3 Hz, 1H), 4.14 (dd, J = 16.1, 8.1 Hz, 1H), 3.67 (brs, 2H), 3.50-3.34 (m, 1H), |
| | 3.15-2.97 (m, 1H), 2.53-2.38 (m, 3H), 2.37-2.11 (m, 2H), 2.04-1.91 (m, 1H), |
| | 1.90-1.74 (m, 2H), 1.72-1.59 (m, 1H), 1.49 (d, J = 6.1 Hz, 3H), 1.38-1.20 (m, |
| | 1H). |
| 13 | ¹H NMR (400 MHz, CDCl₃) δ 5.95 (s, 1H), 4.72-4.60 (m, 1H), 4.52 (td, J = 9.0, |
| | 5.1 Hz, 1H), 4.16 (td, J = 9.0, 7.1 Hz, 1H), 2.51-2.32 (m, 3H), 2.07-1.93 (m, |
| | 1H), 1.61 (s, 2H), 1.51 (d, J = 6.2 Hz, 3H), 0.95 (tt, J = 7.1, 3.4 Hz, 1H). |
| 25 | ¹H NMR: (400 MHz, DMSO-d₆) δ 12.27 (s, 1H), 4.46-4.11 (m, 3H), 3.95-3.66 |
| | (m, 4H), 2.95 (tt, J = 8.0, 5.9 Hz, 1H), 2.80 (dq, J = 8.4, 4.1 Hz, 2H), 2.63 (d, |
| | J = 7.7 Hz, 2H), 2.45-2.35 (m, 2H), 2.29 (td, J = 8.6, 7.5, 4.3 Hz, 1H), 1.87 |
| | (ddt, J = 10.8, 8.9, 7.0 Hz, 1H), 1.40 (d, J = 6.1 Hz, 3H). |
| 26 | ¹H NMR (400 MHz, DMSO-d₆) δ 12.06 (s, 1H), 4.29 (s, 1H), 3.89-3.76 (m, 2H), |
| | 3.75-3.43 (m, 4H), 3.11-3.02 (m, 3H), 2.46-2.25 (m, 4H), 2.22-2.10 (m, 4H), |
| | 1.91-1.81 (m, 2H), 1.47-1.37 (m, 3H). |
| 27 | ¹H NMR (400 MHz, DMSO-d₆) δ 4.27 (dd, J = 13.6, 6.8 Hz, 1H), 3.98 (s, 4H), |
| | 3.88-3.72 (m, 3H), 2.81 (s, 2H), 2.69-2.59 (m, 1H), 2.41 (td, J = 15.0, 7.6 Hz, |
| | 2H), 2.32-2.26 (m, 1H), 2.00 (d, J = 7.9 Hz, 2H), 1.92-1.68 (m, 5H), 1.41 (d, J = |
| | 6.1 Hz, 3H). |
| 28 | ¹H NMR (400 MHz, CD₃OD) δ 4.29-4.12 (m, 7H), 3.72-3.65 (m, 1H), 3.02-2.94 |
| | (m, 1H), 2.90-2.82 (m, 2H), 2.51-2.41 (m, 6H), 1.96-1.93 (m, 3H), 1.53 (d, J = |
| | 6.1 Hz, 3H). |
| 29 | ¹H NMR (400 MHz, DMSO-d₆) δ 8.20 (s, 0.4H), 4.35-3.96 (m, 6H), 3.58 (dd, J = |
| | 9.1, 5.8 Hz, 1H), 3.24 (t, J = 6.1 Hz, 1H), 2.96 (q, J = 8.1 Hz, 1H), 2.85-2.75 |
| | (m, 2H), 2.38 (d, J = 33.1 Hz, 9H), 1.44 (d, J = 6.2 Hz, 3H). |
| 30 | ¹H NMR (400 MHz, DMSO-d₆) δ 4.73-3.94 (m, 8H), 3.92-3.86 (m, 1H), |
| | 3.82-3.72 (m, 1H), 3.02-2.86 (m, 3H), 2.63-2.52 (m, 3H), 2.49-2.41 (m, 2H), |
| | 2.41-2.31 (m, 2H), 1.47 (d, J = 6.5 Hz, 3H). |
| 31 | ¹H NMR (400 MHz, DMSO-d₆) δ 12.12 (s, 1H), 5.51 (d, J = 6.3 Hz, 1H), |
| | 4.30-4.09 (m, 3H), 4.09-4.00 (m, 2H), 3.97 (dd, J = 11.2, 5.9 Hz, 1H), 3.93-3.85 (m, |
| | 1H), 3.49 (dd, J = 8.3, 4.9 Hz, 1H), 3.01-2.89 (m, 1H), 2.81 (s, 2H), 2.47-2.30 |
| | (m, 6H), 1.39 (d, J = 6.3 Hz, 3H). |
| 32 | ¹H NMR (400 MHz, CD₃OD) δ 12.24 (s, 1H), 8.27 (d, J = 4.4 Hz, 1H), 4.27 (dd, |
| | J = 14.0, 6.4 Hz, 1H), 3.95-3.68 (m, 4H), 3.57 (s, 1H), 3.29-3.14 (m, 2H), 3.05 |
| | (d, J = 6.1 Hz, 2H), 2.46-2.24 (m, 5H), 2.07 (d, J = 5.9 Hz, 1H), 1.88 (ddd, J = |
| | 8.5, 7.0, 4.4 Hz, 1H), 1.58 (s, 1H), 1.42 (d, J = 6.2 Hz, 3H). |
| 33 | ¹H NMR (400 MHz, CD₃OD) δ 4.61-4.49 (m, 2H), 4.47-4.38 (m, 1H), 4.06-3.85 |
| | (m, 2H), 3.04-2.91 (m, 4H), 2.53-2.33 (m, 3H), 2.13-2.00 (m, 3H), 1.97-1.89 (m, |
| | 1H), 1.85-1.76 (m, 2H), 1.47 (d, J = 6.2 Hz, 3H), 1.31-1.17 (m, 2H). |
| 34 | ¹H NMR (400 MHz, CD₃OD) δ 4.47-4.37 (m, 1H), 4.26 (s, 2H), 4.12 (s, 2H), |
| | 4.01 (td, J = 8.8, 4.8 Hz, 1H), 3.88 (td, J = 8.8, 7.2 Hz, 1H), 2.88-2.82 (m, 2H), |
| | 2.59-2.34 (m, 8H), 2.02-1.88 (m, 3H), 1.47 (d, J = 6.4 Hz, 3H). |
| 35 | ¹H NMR (400 MHz, CD₃OD) δ 4.29 (q, J = 6.7 Hz, 1H), 3.88-3.78 (m, 4H), 3.17 |
| | (d, J = 3.0 Hz, 3H), 3.03 (td, J = 7.9, 6.4, 3.9 Hz, 2H), 2.87 (d, J = 6.2 Hz, 4H), |
| | 2.69 (d, J = 6.4 Hz, 2H), 2.45-2.24 (m, 3H), 2.12-1.82 (m, 2H), 1.42 (d, J = 6.2 |
| | Hz, 3H). |
| 36 | ¹H NMR (400 MHz, CD₃OD) δ 4.47-4.37 (m, 1H), 4.05-3.95 (m, 2H), 3.89 (dd, |
| | J = 16.1, 8.8 Hz, 2H), 3.67 (s, 1H), 3.33 (s, 1H), 3.14-3.06 (m, 2H), 2.60 (d, J = |
| | 7.6 Hz, 1H), 2.49-2.33 (m, 5H), 2.17 (s, 1H), 1.97-1.87 (m, 1H), 1.66 (d, J = 10.3 |
| | Hz, 1H), 1.51-1.44 (m, 3H). |
| 39 | ¹H NMR (400 MHz, CD₃OD) δ 4.44-4.33 (m, 1H), 4.00 (dt, J = 8.7, 7.5 Hz, |
| | 3H), 3.87 (dd, J = 16.1, 8.7 Hz, 1H), 3.65 (d, J = 11.1 Hz, 2H), 3.05 (td, J = 8.0, |
| | 3.9 Hz, 2H), 2.49-2.31 (m, 3H), 1.98-1.88 (m, 1H), 1.59 (s, 2H), 1.47 (d, J = |
| | 6.2 Hz, 3H), 0.94-0.85 (m, 1H). |
| 41 | ¹H NMR (400 MHz, CD₃OD) δ 8.37-8.31 (m, 1H), 8.12 (d, J = 2.1 Hz, 1H), 4.83 |
| | (dd, J = 14.2, 6.2 Hz, 2H), 4.64 (dd, J = 14.8, 9.6 Hz, 1H), 4.38 (dd, J = 16.5, 9.6 |
| | Hz, 1H), 3.81-3.75 (m, 1H), 3.63 (s, 1H), 3.53-3.45 (m, 1H), 3.23-3.15 (m, 1H), |
| | 2.77-2.69 (m, 1H), 2.62-2.53 (m, 1H), 2.38 (dd, J = 7.5, 3.0 Hz, 2H), 2.29-2.20 |
| | (m, 1H), 2.12-2.03 (m, 1H), 1.82-1.71 (m, 1H), 1.62 (d, J = 6.2 Hz, 3H). |
| 45 | ¹H NMR (400 MHz, DMSO-d₆) δ 7.65 (s, 1H), 5.64 (s, 1H), 4.51 (dd, J = 13.4, |
| | 6.9 Hz, 1H), 4.17 (dd, J = 8.8, 4.2 Hz, 1H), 4.02 (d, J = 7.9 Hz, 1H), 3.66 (d, J = |
| | 9.9 Hz, 1H), 3.61 (d, J = 6.3 Hz, 4H), 3.23 (s, 3H), 2.35 (d, J = 12.5 Hz, 1H), 2.08 |
| | (d, J = 5.3 Hz, 2H), 2.01-1.92 (m, 1H), 1.52 (d, J = 6.1 Hz, 3H), 1.44 (s, 2H), |
| | 0.83 (s, 1H). |

### Biological Test and Evaluation

### (KHK—C ADP-Glo Kinase Inhibitory Activity Assay)

### I. Experimental Procedures:

1) The compounds were transferred to a 384 reaction plates (PE, 6007290) with Echo (Labcyte, 550);
2) After centrifugation, a KHK—C buffer containing 1 nM KHK—C(Origene, TP323488) was added, and incubation was conducted for 15 min at 25° C.;
3) A substrate mixture containing 200 μM D-Fructose (Sigma, F2543) and 100 μM ATP was then added, and incubation was conducted for 60 min at 25° C.;
4) 10 μL of ADP-Glo (Promega, V9102) was added, incubation was continued for 60 mn, then 20 μL of Detection solution was added;
5) After the incubation was continued for 60 min, the luminescence value was read with the Envision multifunctional microplate reader; and
6) Finally, the IC₅₀ (median inhibitory concentration) of each compound was obtained using the nonlinear fitting formula by means of the XLFIT software.

Y=Bottom+(Top−Bottom)/(1+10\hat{ }((LogIC50−X)×HillSlope))
X: Log value of compound concentration
Y: Inhibition ratio (%)

$$Inhibition ratio ( \% ) = \frac{( Mean value of negative control - Reading of compound )}{( Mean value of negative control - Mean value of positive control )} * 100 \%$$

Negative control: DMSO

### II. Experimental Results:

| Example No. | KHK-C (IC₅₀ nM) |
|---|---|
| 1 | 27.86 |
| 2 | 142.53 |
| 3 | 114.72 |
| 4 | 198.28 |
| 5 | 110.05 |
| 6 | 19.05 |
| 7 | 35.98 |
| 8 | 0.17 |
| 9 | 27.00 |
| 10 | 12.64 |
| 11 | 12.55 |
| 12 | NT |
| 13 | NT |
| 14 | NT |
| 15 | 212.48 |
| 16 | 6304.51 |
| 17 | >10000 |
| 18 | 100.15 |
| 19 | 24.74 |
| 20 | 15.44 |
| 21 | 81.25 |
| 22 | 0.44 |
| 23 | NT |
| 22 and 23 | 0.39 |
| Mixture | |
| 24 | 78.35 |
| 25 | 47.02 |
| 26 | 42.93 |
| 27 | 58.99 |
| 28 | >10000 |
| 29 | >10000 |
| 30 | >10000 |
| 31 | 133.55 |
| 32 | 10.38 |
| 33 | 52.78 |
| 34 | 39.95 |
| 35 | 142.23 |
| 36 | 13.60 |
| 37 | 33.27 |
| 38 | 7.29 |
| 39 | NT |
| 40 | 17.26 |
| 41 | 3936.55 |
| 42 | NT |
| 43 | NT |
| 44 | NT |
| 45 | NT |
| 46 | 30.19 |
| 47 | NT |
| Reference 1 | 10.54 |
| | (Document value: 8.4*) |
| Reference 2 | Document value: 1.5* |

Note:
1. ″NT″, i.e., ″Not Tested″, means that the compound was not tested.
2. Reference 1: WO2017115205A1-EX-4(PF-06835919), with a structure
as follows: 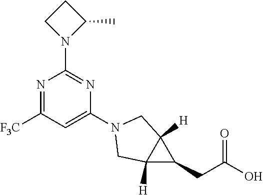 [SMILES: C[C@H]1CCN1c1nc(N2C[C@@H]3[C@@H](CC(=O)O)[C@@H]3C2)cc(C(F)(F)F)n1]
3. Reference 2: WO2017115205A1-EX-1, with a structure as follows: 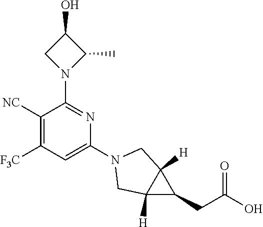 [SMILES: C[C@H]1[C@H](O)CN1c1nc(N2C[C@@H]3[C@@H](CC(=O)O)[C@@H]3C2)cc(C(F)(F)F)c1C#N]
4. The document refers to Patent WO2017115205A1 published by Pfizer.

From the above experimental results, it can be seen that some of the compounds provided in the examples of the present invention can inhibit the KHK—C kinase activity very effectively, with a 5-10-fold increase with respect to Comparative Compound 1 or Comparative Compound 2. These compounds are clinically expected to be developed into more effective KHK—C kinase inhibitors.

All documents mentioned in the present application are hereby incorporated by reference in their entireties, just as each document is cited separately as a reference. In addition, it should be understood that various modifications and changes may be made by those skilled in the art after reading the above disclosure of the present invention, and these equivalent forms also fall within the scope defined by the claims appended hereto.

1. A compound of formula (I), a stereoisomer or pharmaceutically acceptable salt thereof: 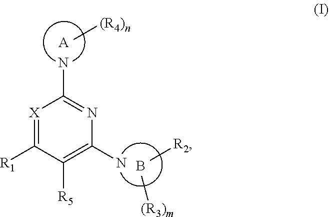
wherein:
Ring A is 4-6 membered nitrogen-containing heteromonocyclyl or 7-12 membered nitrogen-containing heterobicyclyl, and the bicyclyl is a spiro, fused, or bridged ring;
Ring B is 4-6 membered nitrogen-containing heteromonocyclyl or 7-12 membered nitrogen-containing heterobicyclyl, and the bicyclyl is a spiro, fused, or bridged ring;
X is N or CRₐ; Rₐ is selected from the group consisting of hydrogen, deuterium, cyano, trifluoromethyl, and aminoacyl;
R₁ is trifluoromethyl, or, R₁ and Rₐ, together with the moiety directly attached thereto, form structures as follows: 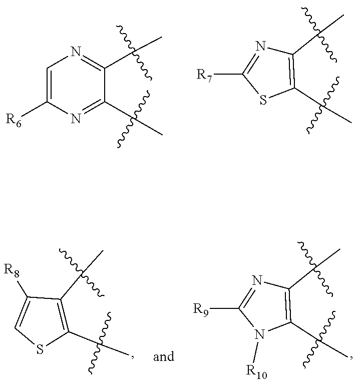
or, R₁ and R₅, together with the moiety directly attached thereto, form a structure as follows: 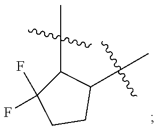 [SMILES: *C1CCC(F)(F)C1*]
R₂ is hydrogen or -L-C(O)—R; L is a bond or C₁₋₆ alkylene, the above C₁₋₆ alkylene is optionally further substituted by a substituent selected from the group consisting of deuterium, halogen, and C₁₋₆ alkyl; R is selected from the group consisting of hydroxy, amino, hydroxyamino, C₁₋₆ alkylamino, C₁₋₆ alkyl, and C₁₋₆ alkoxy;
each R₃ is independently hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl the above C₁₋₆ alkyl, C₁₋₆ alkoxy, C₁₋₆ alkylamino, and C₁₋₆ alkylacyl are optionally further substituted by substituents selected from the group consisting of deuterium, hydroxy, amino, halogen, nitro, cyano, carboxyl, C₁₋₆ alkyl, C₁₋₆ alkoxy, and C₁₋₆ alkylamino;
Rₛ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl;
R₆, R₇, R₈, and R₉ are each independently selected from the group consisting of hydrogen, deuterium, hydroxy, amino, cyano, aminoacyl, acetamido, sulfonyl, methylsulfonyl, isopropylsulfonyl, aminosulfonyl, halogen, nitro, carboxyl, C₁₋₆ alkyl, C₁₋₆ alkoxy, C₁₋₆ alkylamino, and C₁₋₆ alkylacyl;
R₁₀ is selected from the group consisting of hydrogen, deuterium, hydroxy, C₁₋₆ alkyl, and C₁₋₆ alkylacyl;
m is 0, 1, 2, 3, 4, 5, or 6;
n is 0, 1, 2, 3, or 4.

2. The compound of formula (I), the stereoisomer or pharmaceutically acceptable salt thereof according to claim 1, characterized in that Ring B is selected from structures as follows:

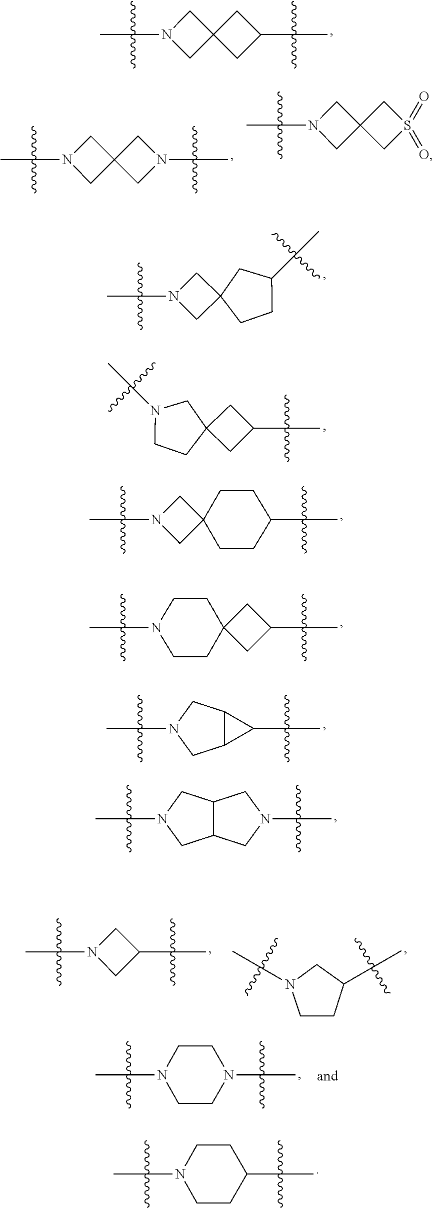

3. The compound of formula (I), the stereoisomer or pharmaceutically acceptable salt thereof according to claim 1, characterized in that the compound of formula (I) is a compound with a structure as shown in the following formula (IIa): 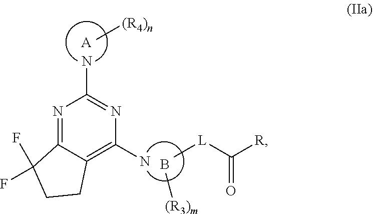
Ring A is 4-6 membered nitrogen-containing heteromonocyclyl, the 4-6 membered nitrogen-containing heteromonocyclyl is selected from the group consisting of azetidine, tetrahydropyrrole, morpholine, piperidine, piperazine, and quinuclidine;
Ring B is selected from the group consisting of 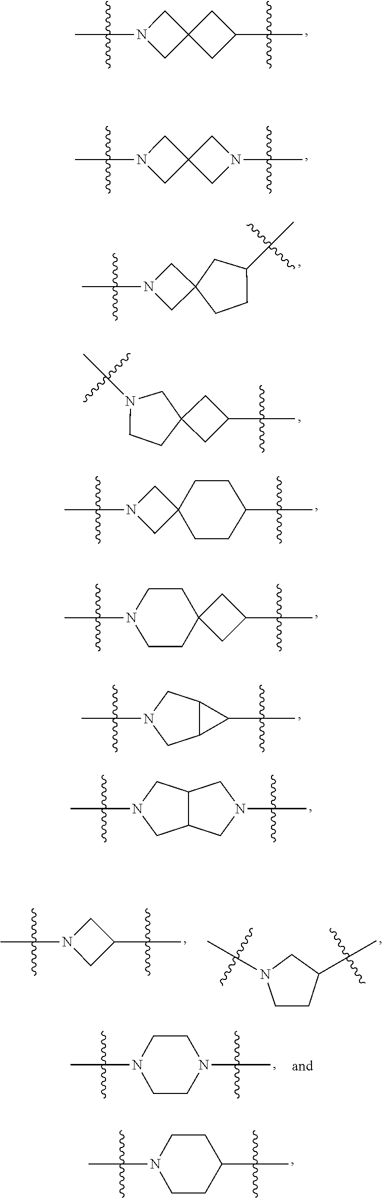
provided that, when Ring B is selected from 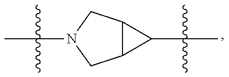 [SMILES: *C1C2CN(*)CC12]
R is hydroxyamino;
L is a bond or C₁₋₃ alkylene;
R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently selected from hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
wherein,
m is 0, 1, 2, 3, 4, 5, or 6:
n is 0, 1, 2, 3, or 4.

4. The compound of formula (I), the stereoisomer or pharmaceutically acceptable salt thereof according to claim 3, characterized in that the compound of formula (I) is a compound with a structure as shown in the following formula (IIIa): 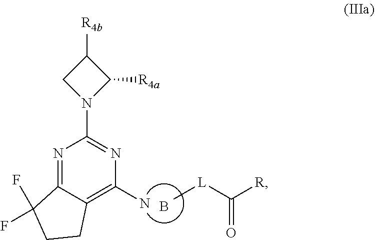
wherein, Ring B is selected from the group consisting of 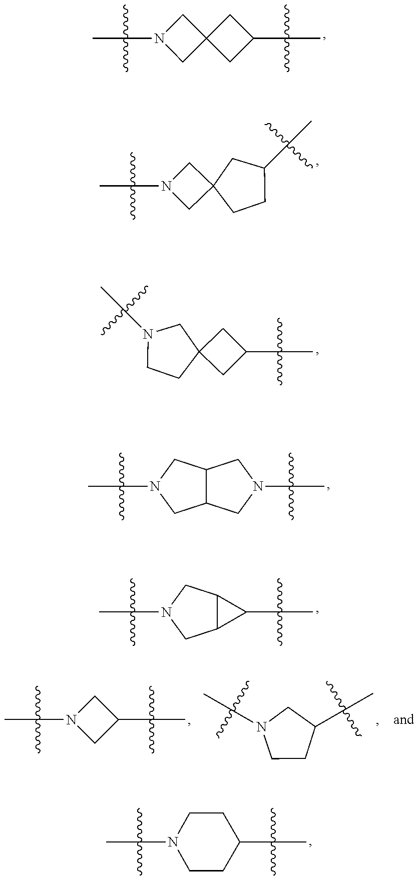
provided that, when Ring B is selected from 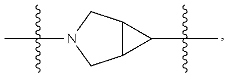 [SMILES: *C1C2CN(*)CC12]
R is hydroxyamino;
L is a bond, methylene, or ethylidene;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl.

5. The compound of formula (I), the stereoisomer or pharmaceutically acceptable salt thereof according to claim 1, characterized in that the compound of formula (I) is a compound with a structure as shown in the following formula (IIb): 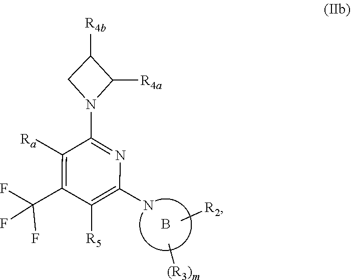
wherein, Rₐ is selected from the group consisting of hydrogen, deuterium, cyano, trifluoromethyl, and aminoacyl;
Ring B is selected from the group consisting of 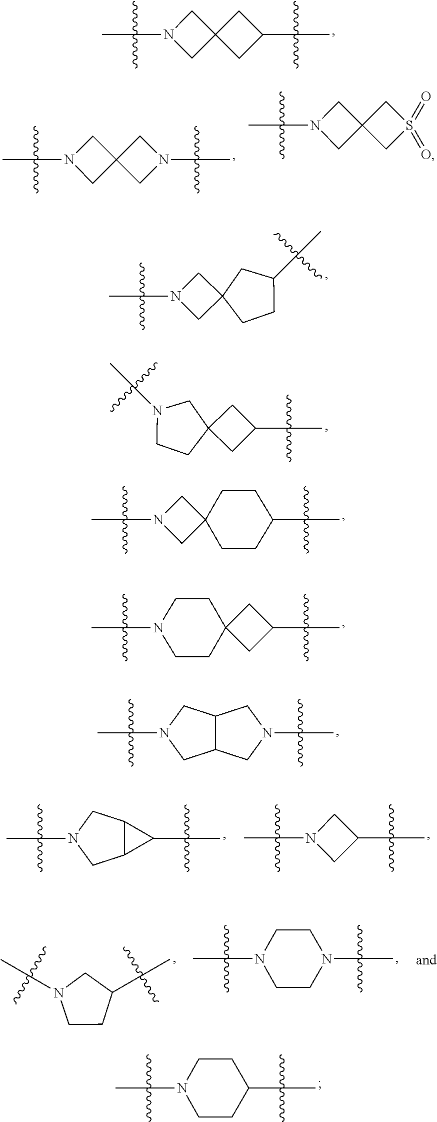
R₂ is hydrogen or -L-C(O)—R;
L is a bond, methylene, or ethylidene, the above methylene or ethylidene is optionally further substituted by a substituent selected from the group consisting of deuterium, methyl, ethyl, and isopropyl, provided that,
1) when L is unsubstituted methylene, Ring B is not selected from 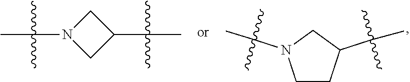 [SMILES: *C1CN(*)C1 ; *C1CCN(*)C1]
or,
2) when L is unsubstituted methylene or unsubstituted ethylidene and Ring B is 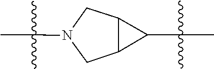 [SMILES: *C1C2CN(*)CC12]
R₄ₐ is methyl or deuteriomethyl and R_{4b} is hydrogen;
R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently hydrogen, deuterium, or fluorine;
R₄ₐ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R_{4b} is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁-3 alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₅ is selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁-3 alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
wherein,
m is 0, 1, 2, 3, 4, 5, or 6.

6. The compound of formula (I), the stereoisomer or pharmaceutically acceptable salt thereof according to claim 5, characterized in that L is a bond, methylene, or ethylidene, the above methylene or ethylidene is optionally further substituted by a substituent selected from the group consisting of deuterium, methyl, ethyl, and isopropyl, provided that,
1) when L is unsubstituted methylene, Ring B is not selected from 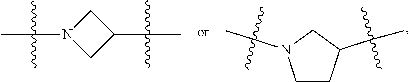 [SMILES: *C1CN(*)C1 ; *C1CCN(*)C1]
2) when L is unsubstituted methylene or unsubstituted ethylidene and Ring B is 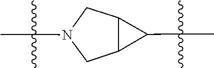 [SMILES: *C1C2CN(*)CC12]
R₄ₐ is methyl or deuteriomethyl and R_{4b} is hydrogen;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl.

7. The compound of formula (I), the stereoisomer or pharmaceutically acceptable salt thereof according to claim 5, characterized in that the compound of formula (I) is a compound with a structure as shown in the following formula (IIIb₁): 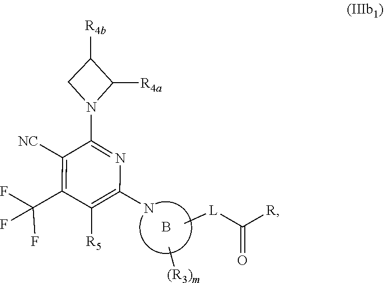
wherein, Ring B is selected from the group consisting of 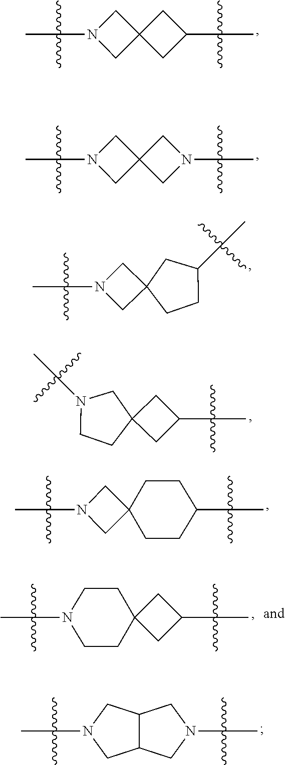
L is a bond, methylene, or ethylidene;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
each R₃ is independently hydrogen, deuterium, or fluorine;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from hydrogen and deuterium;
m is 0, 1, 2, 3, 4, 5, or 6.

8. The compound of formula (I), the stereoisomer or pharmaceutically acceptable salt thereof according to claim 5, characterized in that the compound of formula (I) is a compound with a structure as shown in the following formula (IIIb₂): 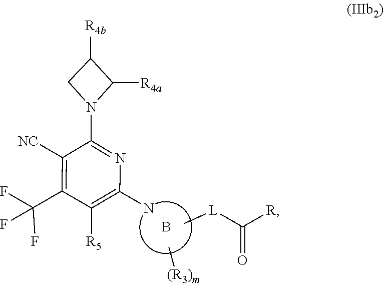
wherein, Ring B is selected from the group consisting of 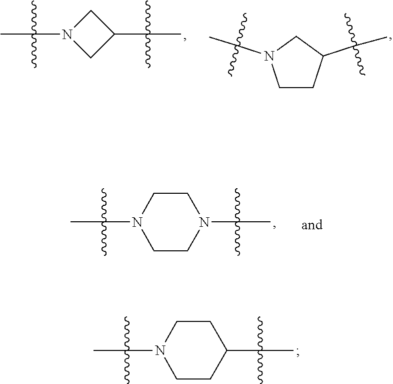 [SMILES: *C1CN(*)C1 ; *C1CCN(*)C1 ; *N1CCN(*)CC1 ; *C1CCN(*)CC1]
L is ethylidene;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
each R₃ is independently hydrogen, deuterium, or fluorine;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₅ is selected from hydrogen and deuterium;
m is 0, 1, 2, 3, 4, 5, or 6.

9. The compound of formula (I), the stereoisomer or pharmaceutically acceptable salt thereof according to claim 5, characterized in that the compound of formula (I) is a compound with a structure as shown in the following formula (IIIb₃): 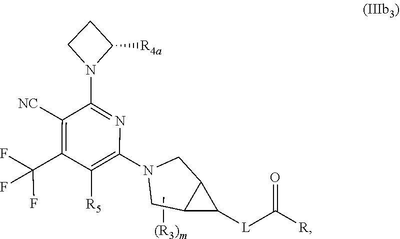
wherein,
L is a bond, methylene, ethylidene, methylomethylene, deuteriomethylene, and deuterioethylidene;
R is selected from the group consisting of hydroxy, amino, hydroxyamino, methoxy, ethoxy, isopropoxy, trifluoromethoxy, and trideuteriomethoxy;
each R₃ is independently hydrogen, deuterium, or fluorine;
R₄ₐ is methyl and trideuteriomethyl;
R₅ is hydrogen and deuterium;
m is 0, 1, 2, 3, 4, 5, or 6.

10. The compound of formula (I), the stereoisomer or pharmaceutically acceptable salt thereof according to claim 9, characterized in that L, R₃, R₄ₐ, and R₅ comprise at least one deuterium atom; preferably, L, R₃, R₄ₐ, and R₅ comprise 1, 2, 3, 4, 5, and 6 deuterium atoms.

11. The compound of formula (I), the stereoisomer or pharmaceutically acceptable salt thereof according to claim 1, characterized in that the compound of formula (I) is a compound with a structure as shown in the following formula (IIc): 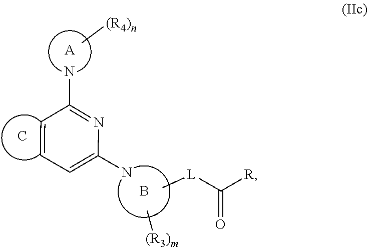
wherein, Ring C is selected from structures as follows: 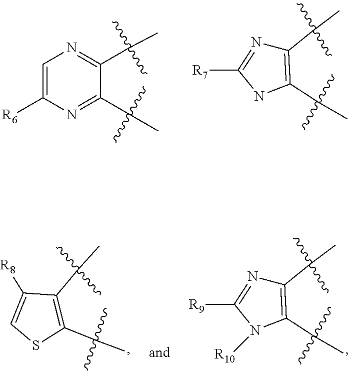
Ring A is 4-6 membered nitrogen-containing heteromonocyclyl, the 4-6 membered nitrogen-containing heteromonocyclyl is selected from the group consisting of azetidine, tetrahydropyrrole, morpholine, piperidine, piperazine, and quinuclidine;
Ring B is selected from structures as follows: 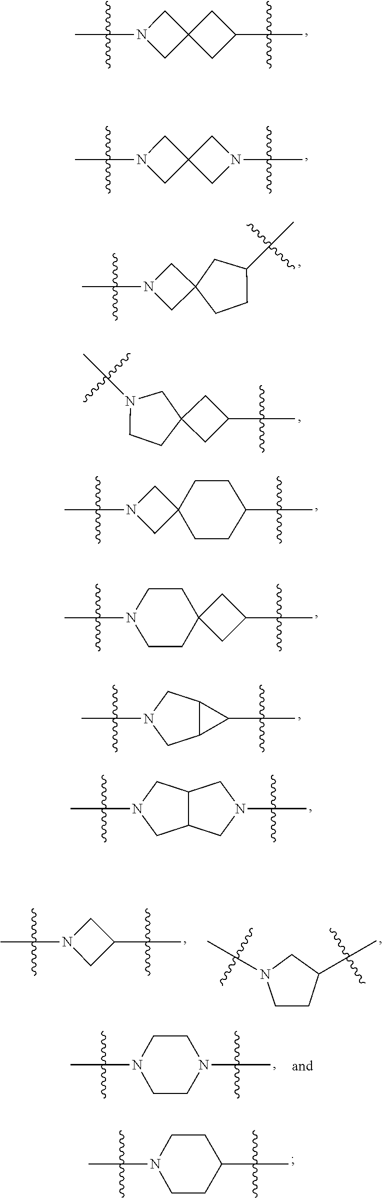
L is a bond or C₁₋₃ alkylene;
R is selected from the group consisting of hydroxy, amino, hydroxyamino, C₁₋₃ alkylamino, C₁₋₃ hydroxyoalkylamino, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy and C₁₋₃ aminoalkyl;
each R₃ is independently hydrogen, deuterium, or fluorine;
each R₄ is independently selected from the group consisting of hydrogen, deuterium, hydroxy, amino, cyano, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₆, R₇, R₈, and R₉ are each independently selected from the group consisting of hydrogen, deuterium, hydroxy, amino, cyano, aminoacyl, acetamido, sulfonyl, methylsulfonyl, isopropylsulfonyl, aminosulfonyl, halogen, nitro, carboxyl, C₁₋₃ alkyl, C₁₋₃ haloalkyl, C₁₋₃ deuterioalkyl, C₁₋₃ alkoxy, C₁₋₃ haloalkoxy, C₁₋₃ deuterioalkoxy, C₁₋₃ alkylacyl, C₁₋₃ aminoalkyl, and C₁₋₃ alkylamino;
R₁₀ is selected from the group consisting of hydrogen, deuterium, hydroxy, C₁₋₃ alkyl, C₁₋₃ haloalkyl, C₁₋₃ deuterioalkyl, and C₁₋₃ alkylacyl;
wherein,
m is 0, 1, 2, 3, 4, 5, or 6;
n is 0, 1, 2, 3, or 4.

12. The compound of formula (I), the stereoisomer or pharmaceutically acceptable salt thereof according to claim 11, characterized in that the compound of formula (I) is a compound with a structure as shown in the following formula (IIIc): 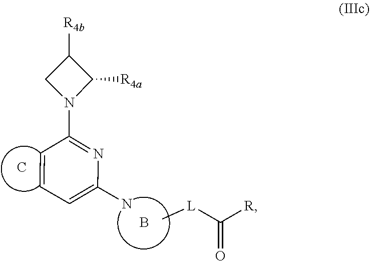
wherein, Ring C is selected from structures as follows: 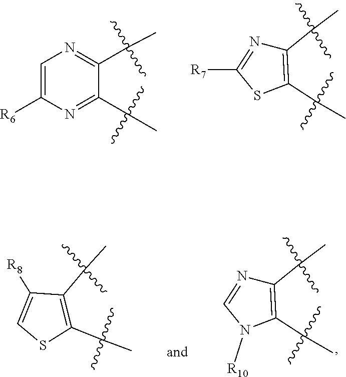
Ring B is 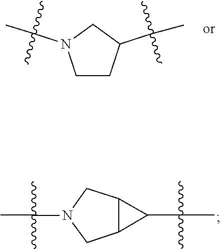 [SMILES: *C1CCN(*)C1 ; *C1C2CN(*)CC12]
L is a bond, methylene, or ethylidene;
R is selected from the group consisting of hydroxy, amino, hydroxyamino, methylamino, ethylamino, hydroxymethylamino, hydroxyethylamino, dimethylamino, methylethylamino, methyl, ethyl, isopropyl, trifluoromethyl, trideuteriomethyl, methoxy, ethoxy, isopropoxy, trifluoromethoxy, and trideuteriomethoxy;
R₄ₐ is selected from the group consisting of hydrogen, deuterium, methyl, ethyl, isopropyl, trifluoromethyl, and trideuteriomethyl;
R_{4b} is selected from the group consisting of hydrogen, deuterium, hydroxy, amino, cyano, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₆, R₇, and R₈ are each independently selected from the group consisting of hydrogen, deuterium, hydroxy, amino, cyano, aminoacyl, acetamido, sulfonyl, methylsulfonyl, isopropylsulfonyl, aminosulfonyl, fluorine, cholorine, carboxyl, methyl, ethyl, isopropyl, trifluoromethyl, trideuteriomethyl, methoxy, ethoxy, isopropoxy, trifluoromethoxy, trideuteriomethoxy, methylamino, ethylamino, dimethylamino, methylethylamino, and acetyl;
R₁₀ is selected from the group consisting of hydrogen, deuterium, hydroxy, methyl, ethyl, isopropyl, trifluoromethyl, trideuteriomethyl, and acetyl.

13. The compound of formula (I), the stereoisomer or pharmaceutically acceptable salt thereof according to claim 1, characterized in that the compound is selected from compounds as follows:

[chemical structure]

[chemical structure]

14. A pharmaceutical composition, comprising the compound of formula (I), the stereoisomer or pharmaceutically acceptable salt thereof according to claim 1, and one or more pharmaceutically acceptable carriers.

15. Use of the compound of formula (I), the stereoisomer or pharmaceutically acceptable salt thereof according to claim 1 in preparation of a medicament for treatment and/or prevention of KHK-mediated diseases.

16. The use according to claim 15, characterized in that the KHK-mediated diseases are selected from the group consisting of endocrine disorders, urological disorders, metabolic disorders, non-alcoholic steatohepatitis, cirrhosis, fatty liver, hepatitis, liver failure, hereditary fructose intolerance, non-alcoholic fatty liver disease, hepatobiliary disorders, fibrotic disorders, cardiovascular and cerebrovascular disorders, immunoinflammatory disorders, central nervous system disorders, gastrointestinal disorders, and hyperproliferative disorders (e.g., cancer).
